(19) United States
(12) Patent Application Publication
(10) Pub. No.: US 2016/0085944 A1
(43) Pub. Date: Mar. 24, 2016

(54) GLASS COATING SPECIFICATION LIBRARY

(71) Applicant: Enki Technology, Inc., San Jose, CA (US)

(72) Inventors: Brenor L. Brophy, San Jose, CA (US); Kevin Kopczynski, Woodside, CA (US)

(21) Appl. No.: 14/489,790

(22) Filed: Sep. 18, 2014

(51) Int. Cl. G06F 19/00; G01W 1/00; G01N 33/38

(52) U.S. Cl. CPC G06F 19/702; G01N 33/386; G01W 1/00

(57) ABSTRACT

Disclosed herein are methods for characterizing environmental factors that affect glass substrates and then based on those factors, determining the optimal coatings to be applied to glass substrates used in solar energy modules and the like to enhance efficiency, general performance and to reduce operational and maintenance costs. Also disclosed are methods and apparatus for applying coatings to flat substrates including substrate pre-treatment processes, coating processes including flow coating and roll coating; coating curing processes including skin-curing using hot-air knives. Also disclosed are coating compositions and formulations for highly tunable, durable, highly abrasion resistant functionalized anti-reflective coatings.

### STATEMENT REGARDING GOVERNMENT FUNDING

This invention was made with government support under Contract DE-EE0006040 awarded by the U.S. Department of Energy. The government has certain rights in the invention.

### BACKGROUND

1. Field

The present disclosure relates generally to the field of thin-film sol-gel coatings and in particular to coating on substrates such as glass or solar modules.

2. Description of Related Art

Coatings have been developed for solar modules, light collectors, building glass and the like that have a variety of beneficial properties, including anti-reflective (AR) properties, hydrophobic and/or hydrophilic properties, abrasion resistant properties, and other properties. Different applications that use such coatings have widely varying requirements, at the manufacturing stage and once installed or otherwise in use in any of a variety of environments. For example, solar glass deployed in harsh environments may benefit from a high degree of abrasion resistance. Designing or selecting an appropriate coating for a particular application is a complex process, requiring consideration of many variables and parameters. A need exists for methods and systems for assisting in the selection and or design of optimal coatings for any of a wide range of applications and situations.

Anti-reflective coatings are used in a wide variety of commercial applications ranging from sunglasses, windows, car windshields, camera lenses, solar modules, and architectural systems. These coatings minimize the reflections on the surface of the glass as the light rays travel through a discontinuous dielectric gradient. The reflection of light usually results in reduced transmittance of the light across the transparent material. For optical applications, it is important that a majority of incident light passes through the interface for maximum efficiency. In this context, anti-reflective coatings provide a useful benefit in optical applications.

Anti-reflective coatings are normally used on glasses, acrylics, and other transparent materials that serve as windows and glass modules associated with architectural structures or energy generating and saving systems, such as solar modules. In building windows, they are used to maximize influx of incident light to maintain proper lighting or natural ambience as well as to minimize distracting reflections from glass surfaces.

In energy generating and saving devices, such as solar modules and light collectors, the utility of anti-reflective coatings lies in the enhanced efficiency of these devices due to a greater degree of light transmittance and, therefore, increased energy generation in relation to the additional cost of the antireflective coating. It is common that solar modules are warranted by their manufacturers to provide energy for a period of 20 or more years. Therefore it is the expectation of the solar module manufacturers using antireflective coatings that the antireflective coatings will retain their antireflective properties at least for the same time period. The majority of the research in antireflective coatings for solar modules has been focused on maximizing the porosity of a film comprising SiO₂ so that the anti-reflective property is maximized. While maximizing the porosity of the SiO₂ anti-reflective coating increases the anti-reflective property of the coated film, it typically does so by compromising its long-term performance. After the coating manufacturing process, the pores are likely to get filled by contaminants inside the factory causing the refractive index to increase. Furthermore, a predominantly porous anti-reflective SiO₂ coating will have a relatively lower resistance to abrasion than a non-porous or less porous anti-reflective SiO₂ coating.

In order for optical elements to perform optimally, it is necessary that they be relatively free from surface contamination and depositions (e.g., dirt) that may reduce light transmittance and, therefore, performance of the coatings. In particular, for optical elements that are exposed to an outside environment, such as solar modules and building windows, the long-term exposure to chemical and physical elements in the environment usually results in deposition of dirt on the surface of the optical element. The dirt may comprise particles of sand, soil, soot, clay, geological mineral particulates, air-borne aerosols, and organic particles such as pollen, cellular debris, biological and plant-based particulate waste matter, and particulate condensates present in the air. Over time, the deposition of such dirt significantly reduces the optical transparency of the optical element. As a result, there is considerable expenditure of human and financial resources associated with regular cleaning of such optical elements, such as transparent windows and solar modules.

For solar modules, soiling can lead to reduction in power output due to reduced absorption of light of typically about 5%, and in some cases losses of 22% have been reported. The paper “The Effect of Soiling on Large Grid-Connected Photovoltaic Systems in California and the Southwest Region of the United States”, Photovoltaic Energy Conversion, Conference Record of the 2006 IEEE 4th World Conference, May 2006, Vol 2, p 2391-2395, reports an average 5% loss. The paper “Soiling and other optical losses in solar-tracking PV plants in Navarra”, Prog. Photovolt: Res. Appl. 2011; 19:211-217, reports losses of 22%.

Dirt deposited on such optical elements can be classified into two types: physically bound and chemically bound particulate matter. The physically bound particles are loosely held due to weak physical interactions such as physical entanglement, crevice entrapment, and entrapment of particulates within the nanoscale or microscale edges, steps, terraces, balconies, and boundaries on the uneven surface of the optical element, such as a window surface. These particles can be dislodged with moderate energy forces such as wind, air from a mechanical blower, or water flow induced by rain or artificially generated sources of flowing water such as a water hose or nozzle. On the other hand, chemically bound particles are characterized by the presence of chemical interactions between the particles themselves and/or between the particulate matter and the optical element itself. In these cases, removal of these particles becomes difficult and usually requires the use of more powerful physical means such as high pressure water washing or manual scrubbing or both. Alternatively, chemical means such as the application of harsh solvents, surfactants, or detergents to the optical element to free the dirt particles from the surfaces can be used. These dirt removal techniques can cause irreparable damage to the antireflective coatings and diminish the value of any investment in anti-reflective coatings or even render the investment worthless.

As noted, the dirt on environmentally exposed optical elements, such as windows and solar modules, may be somewhat removed based upon natural cleaning phenomenon such as rain. However, rainwater is only effective at removing loosely (physically) held particulate matter and is not able to remove the particulate matter that may be strongly (chemically) bonded to optical element, such as the glass or window surfaces. Furthermore, rain water usually contains dissolved matter and small particles that are absorbed from the environment during the rain drop's descent that can leave a visible film on the substrate when dried.

As such, all externally exposed optical elements, in which the optimal transmission of light is important, require some form of routine cleaning efforts in their ongoing maintenance regimen during service. In fact, the surfaces of these items are also often cleaned during fabrication as well as before they are put into service. In fabrication, the surfaces of these items, such as solar modules, are usually cleaned with water, detergent, and/or other industrial cleaners. As a result, anti-reflective coating materials applied to these optical elements need to be able to withstand the use of normal cleaning agents including detergents, acid, bases, solvents, surfactants, and other abrasives while maintaining their physical integrity and maintain their anti-reflective effect. Abrasion of these coatings over time due to cleaning and the deposition of dirt or other environmental particulate may reduce their performance. Therefore, abrasion resistance is an important consideration for anti-reflective coatings particularly for long-term functional performance of a solar module or building window.

A majority of anti-reflective coatings are based on oxides as preferred materials. Some anti-reflective coatings are made of either a very porous oxide-based coating or, alternatively, are comprised of stacks of different oxides. These oxide materials are chemically reactive with dirt particles by means of hydrogen bonding, electrostatic, and/or covalent interactions depending upon the type of coating material and the dirt particle. Therefore, these oxide-based coatings have a natural affinity to bind molecules on their surfaces. Further, highly porous coatings can physically trap dirt nanoparticles in their porous structure. As a result, current anti-reflective coatings are characterized by an intrinsic affinity for physical and/or chemical interactions with dirt nanoparticles and other chemicals in the environment and suffer from severe disadvantages in maintaining a clean surface over their functional lifetime.

Further, one of the most common issues associated with anti-reflective coatings is their performance over the entire solar spectrum, particularly with respect to solar modules. While there are several anti-reflective coatings that are only effective in a narrow region of the solar spectrum, for maximum energy production it is desirable that anti-reflective coatings perform with high transparency over a broad solar region, e.g., the entire solar region from 300 nm to 1100 nm. It is also desirable to have the thickness of the anti-reflective coatings to be tuned to match the spectral responsivity of the underlying solar cell. Consequently, there exists a need in the art for a coating that can provide the combined benefits of anti-reflective properties, such as a coating that can reduce light reflection and scattering from the applicable optical surface; anti-soiling or self-cleaning properties, such as a coating surface that is resistant to binding and adsorption of dirt particles (e.g., resistant to chemical and physical bonding of dirt particles); abrasion resistant properties, such as stability against normal cleaning agents such as detergents, solvents, surfactants, and other chemical and physical abrasives; and UV stability.

Further, it would be beneficial for such coatings to be mechanically robust by exhibiting strength, abrasion resistance, and hardness sufficient to withstand the impact of physical objects in the environment such as sand, pebbles, leaves, branches, and other naturally occurring objects. It would be beneficial for such coatings to also exhibit mechanical stability such that newly manufactured coatings or films would be less likely to develop cracks and scratches that limit their optimum performance, thereby allowing such coatings to be more effective for a relatively longer term of usage. It will be accepted in the solar industry that soiling of solar modules is a local phenomenon that depends upon the environment where the solar modules are placed. Since the soiling mechanisms are different, the cleaning processes required to clean the solar modules are also different. The cleaning processes employed for cleaning the solar modules are also dependent upon regional and local constraints. In some areas where there is less water available for cleaning dirty solar modules, tightly adhered dust is removed by means of dry brushing which could destroy the antireflective coating and hence render the investment in the antireflective coating worthless after a few cleaning cycles. Areas subject to severe sandstorms could also have antireflective coatings on solar modules destroyed by the abrasive action of sand on the solar modules. Therefore solar module manufacturers needing to protect their investment in antireflective coatings will need to have antireflective coatings that are highly abrasion resistant.

In addition, it would be beneficial for such coatings to be able to withstand other environmental factors or conditions such as heat and humidity and to be chemically non-interactive or inert with respect to gases and other molecules present in the environment, and non-reactive to light, water, acid, bases, and salts. In other words, it is desirable to provide coatings having a chemical structure that reduces the interaction of the coating with exogenous particles (e.g., dirt) to improve the long-term performance of the coating.

The soiling mechanisms of solar modules coated with antireflective coatings and placed in highly urban environments are vastly different from the soiling mechanisms present in dry desert or humid desert environments. Solar modules placed in areas that are prone to soiling due to contamination from nearby agricultural activities might have another mechanism for soiling. Given the widely divergent nature of soiling mechanisms, it would be beneficial to have anti-reflective coatings with self-cleaning and anti-soiling properties that are tuned to work under different soiling conditions.

Furthermore, some solar module manufacturers might wish to trade-off two factors such as higher abrasion resistance for a lower gain in transmission from the anti-reflective coating while some other solar module manufacturers might wish to optimize three factors by providing a balance between transmission gain, resistance to soiling and resistance to abrasion.

Oxide coatings are generally made by vacuum (e.g. sputtering, CVD, ALD) and non-vacuum techniques such as sol-gel. However non-vacuum sol-gel method is more cost effective for large area. Si-based Sol is refers to a homogenous solution that is based on three-dimensional silicone polymeric materials. Generally Si-based sol is prepared by hydrolysis and partial condensation of alkoxy silanes. Thin-film sol-gel coating refers to a technique of coating substrates, such as optical surfaces, windows, solar module surfaces, and the like, using a ‘sol’ that undergoes a ‘gelation’ process wherein the three-dimensional silicone oligomers are cross-linked by condensation of Si—OH functionalities to form a solid thin-film on a substrate. These thin-films often undergo a subsequent curing step to increase mechanical strength and other properties. This curing is often accomplished by heating or irradiating the substrate and coating. Thin-film sol-gel coating is a very versatile process that has many industrial uses such as formation of dielectric layers on semiconductor wafers and water repellent layers on ceramics. There are several well-documented techniques for applying wet sol to substrates, some of which are in widespread industry use and others that have generally been limited to the laboratory. Industrial scale sol-gel coating is most commonly performed by a dip, spray, aerosol deposition, spin, meniscus, slot-die or roller process. There are also several methods used to cure sol-gel thin films including baking in ovens, treatment with microwave, infra-red or ultra-violet radiant energy, and exposure to flowing hot gases. These methods may or may not work in concert with components of the coating that catalyze or otherwise aid the curing process.

In the dip coating process the substrate to be coated is dipped into a tank containing the sol. It is then withdrawn at a process dependent speed. As the substrate is slowly drawn from the sol, the gelation process occurs just above the surface and a thin-film layer forms. Dip coating processes are inherently two sided in that all sides and edges of the substrate are coated. This can be advantageous if complete sol coverage is desired but is disadvantageous if the coating on some portion of the substrate interferes with a later substrate processing step. The dip coating technique requires a tank slightly larger than the substrate, which for large substrates means the tank may hold a large volume of sol. For sols mainly composed of organic solvents this may pose a vapor and flammability hazard. It may also be challenging to control the composition and quality of the sol within the large tank. Each new substrate dipped in the tank may carry contamination that is transferred to the sol; the sol might become depleted in some element as more substrates are processed causing a variation in the thin-film produced. The sol may change through evaporation of solvent at the surface where substrates are introduced.

Spray coating exists in many forms, but generally may be considered to be the deposition of material through a nozzle under pressure or the atomization of material which is then entrained by a jet of gas. In all cases the material is moved across a gap between a nozzle and a surface to be coated. The purpose of the spray system is to deposit a uniform layer of material over a wide area of the substrate. In the context of sol-gel coatings on substrates spray coating has the advantage of only applying fresh material to the substrate. Careful selection of solvents and control of solvent evaporation is needed to ensure that the correct final concentration of sol is delivered to the substrate. Spraying typically requires that either the nozzle or the substrate is moved in order to coat an area, for example the substrate may be moved past a line of stationary nozzles.

Spin coating is commonly used in the semiconductor wafer processing industry and in the LCD display module industry to apply even layers of material to the surface of flat substrates such as silicon wafers or large pieces of glass. It has the same advantage as spray coating in that only fresh material is deposited. It also has excellent uniformity control. Generally, equipment to perform the spin coating tends to be complex and costly to maintain because of the fine mechanical control needed to achieve uniformity. This is particularly true as the size of the substrate increases.

Meniscus coating was historically used in the semiconductor industry before giving way to spin coating. It remains in use by some equipment vendors in the LCD display industry. Meniscus coating works by passing a substrate to be coated over a narrow slot at a very close distance such that material forced up through the slot forms a continuous meniscus with the substrate. As the substrate moves across the slot this meniscus deposits a layer of material on the substrate. The technique requires fine control over the distance between the slot and the substrate across the full length of the slot. Generally, the substrate must be extremely flat to avoid deviation in this distance. Additionally, this technique works best with viscous materials that can form a large meniscus. This limits its usability with sol-gel formulations that use comparatively low viscosity solvents.

Roll coating is a common application method for sol-gel coatings on flat substrates. In one embodiment of this process, material is deposited from a reservoir onto an application roller. A doctor blade or doctor roller may be used to control the thickness of the coating material placed on the application roller. That material is then transferred directly from the application roller to the substrate. In general, roll coating works best with continuous substrates, such as, for example, a roll of steel. In the case of discontinuous substrates such as pieces of glass or wood, for example, special techniques may be employed to control coating uniformity at the leading and trailing edges of the substrate. These techniques include, for example, varying the application roller contact pressure by having the coating roller touch down on the leading edge and lift-off the trailing edge in a precisely controlled manner. The application roller may run in a forward direction, i.e. rolling with the substrate direction of movement or in a reverse direction, wherein the application roller opposes the direction of movement of the substrate. The surface of the application roller may be made of a compliant material that serves to compensate for any surface or flatness variations on the substrate and to provide a surface to which the coating material will adhere in a reasonably uniform manner, or the application roller may be a comparatively solid material. Depending on the rheology of the material to be coated, the surfaces of the rollers may be patterned with grooves or other textures to add in coating application.

Flow coating is a technique where coating material is flowed over a surface to be coated. The excess drips away and that which remains on the surface forms the final coating. The surface may be flat or irregular. In general, the substrate is oriented such that the coating material flows due to gravity. Advantages of this technique are its simplicity, ability to coat irregular surfaces, and the option to use only fresh material or to recirculate the excess material that drips off the surface.

It would also be preferable to enable drying and curing of such coatings at relatively low temperatures, such as below 150° C. so that the coatings could be applied and dried and cured on substrates to which other temperature sensitive materials had been previously attached, for example a fully assembled solar panel.

Partial curing can occur during drying process which converts the sol to gel form but further thermal curing is needed to densify the coating. One common cure method is to heat a sol-gel coated article in an oven. This has the advantage of simplicity. The oven may be of the batch type wherein a batch of coated material is placed in an oven that is then sealed, and maintained for a period, then opened and the batch removed. While in the oven, the coater material may be subject to a varying temperature profile created by the oven's controller. Alternatively, the oven may be of the continuous type wherein a conveyor belt or similar transport mechanism moves coated articles through a heated container. As the material moves through the container it may experience different temperatures in different zones creating a temperature profile consisting of heating, soaking at a fixed temperature, then cooling. The profile may be a function of the temperature zones within the oven and the speed of the transport mechanism. Heat within the oven may be provided by convection with hot gases created by combustion of fuel gas or by the heating of gas by electrical elements. Alternatively, the coated article might be heated by radiant heat.

Some types of sol-gel coatings may be cured with ultra-violet radiation. In these types of materials, chemical crosslinking within the material is promoted by high-energy photons.

For the curing of thin coatings on surfaces, hot gasses may be passed directly over the thin-film to heat the surface layer by conduction.

Optimal methods for industrial scale sol-gel coating of flat substrates should be capable of selectively coating just one face of a substrate; be economical in their use of the coating material; provide easy compositional and contamination control; be versatile with respect to the sol-gel formulation such that solvents of different volatilities can be used and chemically compatible with critical equipment; be of low complexity and cost; capable of handling large imperfections in substrate surface flatness, and capable of achieving superior coating uniformity. Optimal curing methods should be cost effective; not damage the coated substrate; match the throughput of the prior coating process step and effectively cure the coating material to its final desired properties.

### SUMMARY

The present disclosure provides methods and systems for designing and/or selecting one or more effective coating compositions, coating methods, and/or coated product based on information about the requirements of a particular application, the desired manufacturing process for a particular product, and/or the environment in which a product will be deployed. Such methods and systems allow users to account for a wide range of parameters and input variables in obtaining a preferred coating for a given situation.

The present disclosure further provides coating compositions comprising a gel formed from a sol comprising a polysilsesquioxane, a solvent, optionally a catalyst, and optionally other additives wherein the coating compositions are converted to dry gel upon drying that is subsequently cured to form a coating on a glass substrate having a desired combination of anti-reflective properties, anti-soiling or self-cleaning properties, and abrasion resistance. Accordingly, the anti-reflective coatings provided by the present disclosure are physically, mechanically, structurally, and chemically stable and provide a higher transmission compared to uncoated glass.

Float glass is one type of glass that is a familiar material for architectural and photovoltaic applications. There are some interesting properties of float glass that may be exploited for improving the performance of antireflective coatings. The process of fabrication of float glass by casting molten soda lime glass on a molten tin bath produces float glass which leads to tin diffusion on the face of the glass in contact with molten tin. The other face, which is in contact with inert atmosphere, is weakly contaminated with tin and is called the air-side. Thus float glass is known to be comprised of two composite surface layers and a bulk, one of composite surface being significantly richer in tin concentration compared to the other. It is beneficial to identify and preferentially coat the tin rich side of float glass with an antireflective coating so that amount of light reflected is lower when compared to the amount of reflected light from the same type of antireflective coating applied to the air side of the glass. More specifically, some thin film solar module manufacturers deposit a transparent conducting oxide on the air-side of float glass. Therefore, it is highly beneficial to have an anti-reflective coating that works cooperatively with the tin rich side of float glass to provide all of the anti-reflective coating's advantageous properties.

In some embodiments, the aforementioned coating compositions can be applied to any substrate wherein a higher transmission of light is required compared to an uncoated surface.

In some embodiments, the coating compositions include a combination of sols having the following formula A:

[RSi(OH)O_{1.5}]ₐ[RSiO_{1.5}]_{b}[RSi(OH)O]_{c}[R′Si(OH)O]_{1.5}]ₘ[R′SiO_{1.5}]ₙ[R′Si(OH)O]ₚ[SiO₂]ₓ[Si(OH)O_{1.5}]_{y}[Si(OH)₂O]_{z}

In the Chemical structure A, R is independently hydrogen or a substituted or unsubstituted C1 to C10 alkyl group, a substituted or unsubstituted C3 to C20 cycloalkyl group, a substituted or unsubstituted C1 to C10 hydroxyalkyl group, a substituted or unsubstituted C6 to C20 aryl group, a substituted or unsubstituted C2 to C20 heteroaryl group, a substituted or unsubstituted C2 to C10 alkenyl group, a substituted or unsubstituted carboxyl group, a substituted or unsubstituted glycidylether group, a hydroxyl group, or a combination thereof. R′ is a fluorine substituted C1 to C10 alkyl group, a fluorine substituted C3 to C20 cycloalkyl group, a fluorine substituted C1 to C10 hydroxyalkyl group, a fluorine substituted aryl group, a fluorine substituted C2 to C20 heteroaryl group, a fluorine substituted C2 to C10 alkenyl group, a fluorine substituted carboxyl group, a fluorine substituted glycidylether group, or a combination thereof, 0<a,b,c,x,y,z<0.9, 0≦m,n,p<0.9, and a+b+c+m+n+p+x+y+z=1.

In some embodiments, the composition of the coating composition is based upon a precise selection of solvent, pH, solvent to water ratio, and solvent to silane ratio that allows the resulting sol to remain stable for a significant period of time without exhibiting change in its chemical or physical characteristics. In some embodiments, the composition of the coating composition is based upon controlling the precise amounts and/or ratios of the different silanes in the coating composition. The amount of the silanes in the coating composition can be used to control the final composition and thickness of the antireflective coating while the ratio of the different silanes in the coating composition can be used to tune the abrasion resistance and/or transmission and/or anti-soiling and/or self-cleaning property of the anti-reflective coating.

Some embodiments also provide methods for applying the coatings and for using such coatings. In some embodiments, the methods of treating a substrate comprises pretreatment of the substrate based on combination of chemical treatment, etching, and/or polishing or cleaning steps that enable better interaction of the sol with the surface of the glass for making a coating with thickness ranging from 50 nm to 200 nm. Thereafter, in some embodiments, the methods include applying the sol to the surface of the glass substrate and allowing the sol to gel to form the coating with the desired properties. Pretreatment methods for annealed or tempered, float or patterned glass or other kinds of glass substrates could be of a similar nature. It should be apparent to one skilled in the art that different surface preparation conditions will impact the flow of a coating material on the glass and could lead to films with different thickness and abrasion resistance. In some embodiments, the application of the sol to the substrate includes drop rolling, flow coating and roll coating that result in uniform deposition of the sol to form an even, uniform and crack-free coating. In some embodiments, the method includes thermally treating the coated articles under specific condition of heat and humidity to form a chemically durable coating that adheres strongly to the substrate without cracking and/or peeling.

In some embodiments, the disclosure provides for the use of the coating compositions as an efficiency enhancement aid in architectural windows in building and houses by the provision of anti-reflection benefits and/or by the provision of anti-soiling benefits to augment the anti-reflection benefits. In other embodiments, the disclosure provides for the use of the coating compositions as an efficiency enhancement aid in treatment of transparent surfaces (that require regular cleaning) to make them self-cleaning.

In some embodiments, the disclosure provides a coated glass-based article suitable for use as outer cover of a solar module assembly that is anti-reflective, hydrophobic and/or oleophobic and exhibits resistance to abrasion, UV light, heat, humidity, corrosives such as acids, bases, salts, and cleaning agents such as detergents, surfactants, solvents and other abrasives.

In an aspect, a platform for determining a coating for glass or solar modules to account for the interaction of the module with the environment may include one or more of algorithms for determining a coating, instructions for application of the coating, and tools for applying the coating.

In an aspect, a process for determining a coating adapted to the conditions/soiling types that a solar/glass module is in or will be in may include using an algorithm to match one or more coatings from a library to one or more inputs, data points, or process specifications. The algorithm may also determine a sequence for applying the one or more coatings. The algorithm may access a database including data on at least soiling variables and coatings. The database may further include data on performance for optimization of an array given a particular variable, wherein the variable is related to one or more of a location/geography, surface type, product type, soil type, and project economics. The algorithm may be adapted to tune properties of anti-soiling/anti-reflectiveness versus longevity of the module. The algorithm may be adapted to tune properties of anti-soiling/anti-reflectiveness versus cost of capital (for project economics). The algorithm may be adapted to tune the properties of the coating to reduce or improve the performance of the solar module.

In an aspect, a process for determining a coating for a glass or solar module may include taking at least one input, user requirement, or process specification, identifying a candidate coating, wherein identifying includes consulting one or more of a coating library, conditions data, testing data, and module specification, ranking/sorting the candidate coatings, and presenting the candidate coatings in a user interface that allows users to sort the candidate coatings by one or more properties or preferences (e.g. price, AR, durability).

In an aspect, a process for developing a coating adapted to the conditions/soiling types that a solar/glass module is in or will be in, wherein the process includes using an algorithm to generate a coat suggestion based on information about the conditions/soiling types. The suggested coat can be generated by combining coatings from a library of coatings. Upon generating the suggested coat, it may be deposited into a library of coating for future use in a matching process for coatings to modules.

In an aspect, a process for modifying the specification of a solar/glass module at the manufacturing stage may include applying a coating. The coating may be applied to a finished module. The coating may be applied at a particular thickness, wherein the thickness is selected to improve manufacturing tolerances. The coating may increase or decrease the solar energy collection efficiency. The modules may first be binned by performance and the coating may be serially applied to those modules in a particular bin. The coating may be selected so that the modules in the first bin match the performance of the modules in the second bin. The coating may improve or decrease the performance of the module. The coating may be selected so that the modules in the first bin match the impedance of the modules in the second bin.

In an aspect, a method for enabling performance timing of a solar array, may include inputting a schedule of solar module installation into a solar array into a processing system, and determining a coating for each solar module to be installed into the array so that a maximum performance is reached at substantially the same time. The coating on an early set of installed solar modules may be optimized for less than maximum performance at the beginning of application, wherein wear of the coating improves performance over time. The coating on a later set of installed solar modules may be selected to match the performance of the earlier installed solar modules after the coating has been worn/abraded.

In an aspect, a process for activating elements of a coating applied to a glass or solar module at low temperature may involve evaporation of a porogen or application of a heat gun/air knife/UV.

In an aspect, a coating determination user interface may include receiving at least one input, user requirement, or process specification, receiving one or more preferences for data, libraries, and inventory to be searched in identifying a candidate coating, processing the at least one input, user requirement, or process specification in accordance with the preference to identify at least one candidate coating, and displaying the at least one candidate coating in a ranked list that is sortable by one or more properties or fields (e.g. price, AR, durability).

In an aspect, a coating determination user interface may include receiving at least one input, user requirement, or process specification, receiving one or more preferences for data, libraries, and inventory to be searched in identifying a candidate coating, processing the at least one input, user requirement, or process specification in accordance with the preference to identify at least two candidate coatings, and displaying the at least two candidate coatings in a comparison chart that aligns one or more properties or fields of the candidate coatings (e.g. price, AR, durability).

In an aspect, a library of coatings may be available for application to a solar/glass module during or after manufacturing that are each targeted to modifying the module in a specific way. The coatings may be optimized for various applications, such as anti-soiling, anti-reflective, spectral shifting, spectral filtering, etc. For example, spectral filtering may be done in ways that increase efficiency, such as to reflect more IR to keep the panel cooler. The coating may be optimized for different solar conditions including diffuse light (cloudy) or a sunny point source, various dust compositions in different areas, condensation in the morning, etc.

In an aspect, a coating for a solar/glass module available for application to a solar/glass module during or after manufacturing may be directed at reducing the adherence of avian fecal matter to the module.

In an aspect, a coating for a solar/glass module available for application to a solar/glass module during or after manufacturing may be directed at reducing the adherence of biofilms/molds to the module.

In an aspect, a coating for a solar/glass module available for application to a solar/glass module during or after manufacturing may be directed at reducing the damaging effects of acid rain/SO₂ exposure to the module.

In an aspect, a coating for a solar module may be selected to enable re-application of functional layers to a solar module in the field. The coating may include exposed or easily exposable hydroxyl groups as bond forming units for applied silica. The AR coating density may avoid re-application layers entering pores that adversely impact AR. The re-applicable hydrophobic top (fluoroalkylsilane) coating combined with an initial dense AR coating may be used to refresh hydrophobicity over time without adverse impact on AR.

In an aspect, a kit for in-field application of a coating to a solar/glass module may include one or more of a questionnaire for determining a coating, instructions for application of the coating, and tools for applying the coating. The device may be adapted to disperse various coatings to particular thicknesses and in various combinations. The device may be further adapted to cure the coating in situ.

In an aspect, a robotic arm may be controlled by a technician/user for applying a coating in situ to a solar/glass module, wherein the robotic arm is adapted to disperse various coatings to particular thicknesses and in various combinations and sequences. The robotic arm may be further adapted to cure the coating in situ.

In an aspect, a compressed air or vacuum system for cleaning a solar array may include an integrated sensor for determining when the array is in need of cleaning. The system may be powered by the solar array itself.

In an aspect, a computer-based process for selecting a coating specification for a glass to optimize the performance parameters of the glass may include receiving an electronic data structure representing at least one requirements specification for the glass at a computer, and automatically selecting at least one candidate coating specification, wherein selecting includes searching at least one of a coating library, a conditions database, a testing database, and a module specification database based on the at least one requirements specification data structure. Selecting may include identifying at least one coating specification based on a known performance of the coating specification in response to the at least one requirements specification. The process may further include presenting in a user interface a measure of a match between the requirements specification and at least one selected coating specification. The process may further include ranking a plurality of coating specifications based on the extent of match to the requirements specification. The process may further include recommending a coating specification based on the extent of match. The glass may be a component of a solar module or a building. The coating specification may be selected to increase optical transmission through the glass relative to uncoated glass, to reduce soiling on the glass surface relative to uncoated glass, to increase self-cleaning of the glass surface in the presence of running water on the glass surface relative to uncoated glass, to increase the durability of the coating and the glass relative to uncoated glass, from a database of sol-gel coating specifications, and the like. The requirements specification may be at least partially provided by a customer for the coating material. The coating specification may be selected using one or more of the following algorithms: first eliminating coating candidates based on a criteria before further evaluation, evaluating matching criteria in a ranked list, evaluating matching criteria using a weighted list, using a mapping engine or using a matrix. The requirements specification may include at least one of: a location, a use environment, a type of use, an orientation, one or more performance criteria, a glass type, a solar module type, a cost, economic factors, process specifications and process limitations. The coating specification may include at least one of a type of coating material, a coating application process or a coating curing process. The coating specification may be used to generate a quotation for supplying coating material to a customer. The coating specification may be selected by using coating performance data from a database, wherein the coating performance data is for the same environmental classification as the location provided in the requirements specification. The environmental classification may be derived by compilation of environmental data from one or more sources, correlating the data by location, identifying distinct groups of data related to the performance of glass, and creating classification names to represent each distinct group such that glass performance is substantially similar in locations that share the same classification. The coating performance data may be derived by one or more of the following: in situ measurement of coating performance at selected locations with representative environmental classifications, performance data from existing solar installations, and measurement of coating performance user simulated environmental conditions. A user may be presented a ranked list of selected coating specifications. The list may be filtered or sorted based upon the values of particular selection variables. The selected coating specification may be used to generate input data for solar energy system simulator software.

In an aspect, a process for producing a coated solar glass module may include receiving an electronic data structure representing at least one requirements specification for the performance of the solar glass module at a computer, and automatically selecting at least one candidate coating specification, wherein selecting includes searching at least one of a coating library, a conditions database, a testing database, and a module specification database based on the at least one requirements specification data structure, wherein the selected coating specification improves the solar energy collection efficiency of the coated glass as compared to an uncoated glass.

In an aspect, a process for developing a coating specification library may include gathering geo-located environmental data, wherein the geo-located data include at least one of climatic classification data, meteorological data, pollution data, soil classification data and biological data, associating particular variables derived from a set of environmental datasets with geographic locations in a single GIS database by importing each environmental dataset as a set of maps, wherein the maps can be viewed and manipulated as a stack of layers and wherein new layers can be derived by algorithmically combining multiple layers, generating a location genome comprising a set of values for the particular variables associated with the geographic locations, wherein the values describe relevant data for predicting the interactions of glass surfaces with the environment at that geographic location, sorting the locations into a limited number of searchable classification groups based upon their genome values to reduce the very large number of unique location genomes to a smaller number of classes that can then be used to select specific coating properties, and determining the response of glass with and without various coatings at the geographic locations with selected classifications and storing the coating response in association with at least one of the location genome and location classification group in a coating response database. The data may further include at least one of measurements of temperature, precipitation, wind, rainfall, snowpack, river flow, time spent below dew point, solar, spectral distribution, air quality index, specific pollutant concentration, SO₂ concentration, dust type/concentration/dust chemistry, alkali lakebed presence, road salt exposure, iron oxide exposure from train tracks, petrochemical/combustion exposure from nearby industry, tire/break debris from nearby roads, seabird population, insect populations, biofilm/mold prone areas, moss, altitude, rivers, lakes, desert, seaside, and sea foam exposure. The process may further include optimizing the location genome by filtering locations to include only locations that are suitable for the installation of solar energy generation systems. The process may further include optimizing the location genome by ranking locations according to how suitable they are for the installation of solar energy generation systems. The process may further include optimizing the location genome by sorting the specific geographic locations by size. Development of the classification group may be by at least one of theory and empirical evidence from field sensors or lab experiments.

In an aspect, a user interface for determining a coating specification for glass may include a requirements interface element representing at least one requirements specification for the glass at a computer, an access interface element providing access to a set of resources to be searched in identifying one or more candidate coating specifications, and a ranking interface displaying the candidate coating specifications in a ranked list that is sortable by one or more properties or fields (e.g. price, AR, durability). The user interface may include a selection element enabling a user to select one or more of the candidate coating specifications. The access interface may display the candidate coating specifications in a comparison chart that aligns one or more properties or fields of the candidate coatings (e.g. price, AR, durability).

A coating specification library for coating glass may include a GIS database including an electronic data set of geo-located environmental data representing at least one of climatic classification data, meteorological data, pollution data, soil classification data and biological data, wherein at least one, and a location genome electronic data structure comprising a set of values for the particular variables associated with the geographic locations, wherein the values provide relevant data for predicting the interactions of glass surfaces with the environment at a geographic location, wherein the geographic locations are classified into searchable classification groups based upon their genome values. Maps based on the GIS database can be viewed and manipulated as a stack of layers. New layers can be derived by algorithmically combining multiple layers. The coating specification library may further include a module storing data relating to performance of glass using coatings having particular characteristics under particular environmental conditions. The environmental data may further include at least one of measurements of temperature, precipitation, wind, rainfall, snowpack, river flow, time spent below dew point, solar, spectral distribution, air quality index, specific pollutant concentration, SO₂ concentration, dust type/concentration/dust chemistry, alkali lakebed presence, road salt exposure, iron oxide exposure from train tracks, petrochemical/combustion exposure from nearby industry, tire/break debris from nearby roads, seabird population, insect populations, biofilm/mold prone areas, moss, altitude, rivers, lakes, desert, seaside, and sea foam exposure. The location genome may be filtered to include only locations that are suitable for the installation of solar energy generation systems. The location genome may be ranked according to how suitable the locations are for the installation of solar energy generation systems. The location genome may be filtered by sorting the specific geographic locations by size.

These and other systems, methods, objects, features, and advantages of the present disclosure will be apparent to those skilled in the art from the following detailed description of the preferred embodiment and the drawings.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context.

### BRIEF DESCRIPTION OF THE DRAWINGS

The disclosure and the following detailed description of certain embodiments thereof may be understood by reference to the following figures:

FIG. 1a illustrates the UV-vis transmittance spectra comparing the transmission gains of coating made from composition given in Example 2 on tin vs non-tin side of float glass on a 30×30 cm substrate;

FIG. 1b illustrates the UV-vis transmittance spectra of coating made from Example 3 roll coated on patterned glass substrate

FIG. 2a illustrates the UV-vis transmittance spectra showing maximum transmittance enhancement of coatings on tin side of TCO glass substrates made from compositions given in Example 3 with pre- and post-abrasion spectra.

FIGS. 2b and 2c illustrates the UV-vis spectra of coatings on tin side of TCO glass substrate made from composition of example 5 and 7 respectively.

FIG. 3a is TEM cross-sectional view of a coating made from the composition of Example 2 on a glass slide substrate;

FIG. 3b is a High resolution TEM of a coating made from the composition of Example 2 on a glass substrate;

FIG. 4 is an SEM cross-sectional view of a coating made from the composition of Example 2 on a 30×30 cm glass substrate;

FIG. 5 is an SEM cross-sectional view of a coating made from the composition of Example 3 on a 30×30 cm glass substrate;

FIG. 6 is an SEM cross-sectional view of a coating made from the composition of Example 4 on a 30×30 cm glass substrate;

FIG. 7a-1 is a GPC of sol made from Example 2;

FIG. 7a-2 showing the spread of the molecular weights for sol made from Example 2;

FIG. 7b-1 is a GPC of sol made from Example 3;

FIG. 7b-2 showing the spread of the molecular weights for sol made from Example 3;

FIG. 7c-1 is a GPC of sol made from Example 4;

FIG. 7c-2 showing the spread of the molecular weights for sol made from Example 4;

FIG. 7d-1 is a GPC of sol made from Example 5;

FIG. 7d-2 showing the spread of the molecular weights for sol made from Example 5;

FIG. 7e-1 is a GPC of sol made from Example 6;

FIG. 7e-2 showing the spread of the molecular weights for sol made from Example 6;

FIG. 7f-1 is a GPC of sol made from Example 7;

FIG. 7f-2 showing the spread of the molecular weights for sol made from Example 7;

FIG. 7g-1 is a GPC of sol made from Example 8;

FIG. 7g-2 showing the spread of the molecular weights for sol made from Example 8;

FIG. 7h-1 is a GPC of sol made from Example 9;

FIG. 7h-2 showing the spread of the molecular weights for sol made from Example 9;

FIG. 7k-1 is a GPC of sol made from Example 10;

FIG. 7k-2 showing the spread of the molecular weights for sol made from Example 10;

FIG. 8a is the XPS spectrum of coating from Example 2 on tin side of TC of TCO coated glass; and

FIG. 8b is the XPS spectrum of coating from Example 2 on tin side of TCO coated glass after 10 minute Argon Sputter Etch.

FIG. 9 depicts an embodiment of flow coating;

FIG. 10 depicts a cross-sectional view of an embodiment of a flow coating head;

FIG. 11 depicts a cross-sectional view of a second embodiment of a flow coating head;

FIG. 12 depicts an isometric view of a flow coating head lower slot manifold;

FIG. 13 depicts a partial view of the assembled flow coating head of FIG. 10 and a corresponding substrate;

FIG. 14 shows a schematic cross-sectional view of a coating slot identifying several critical dimensions and parameters;

FIGS. 15a and 15b depict a roll-coat system optimized for coating on flat substrates;

FIG. 16 depicts an embodiment of a roll-coat system for flat substrates;

FIG. 17 depicts an embodiment of a skin-cure system.

FIG. 18 depicts an example temperature profile for a skin-cure system.

FIG. 19 depicts an example of thermogravimetric analysis of representative samples of coating material.

FIGS. 20a, 20b and 20c show data for an exemplary sol-gel coating that demonstrate control of final film thickness, refractive index and water contact angle as a function of maximum cure temperature.

FIGS. 20d and 20e depicts an example of FT-IR analysis of representative samples of coating material before and after the curing process.

FIGS. 21 and 22 depicts Si-NMR spectra of example 2 and 3 respectively.

FIG. 23 depicts a platform for prescriptive coatings.

FIG. 24 depicts a sample workflow of the platform for determining a custom coating for glass or solar modules.

### DETAILED DESCRIPTION

Various embodiments of the disclosure are described below in conjunction with the Figures; however, this description should not be viewed as limiting the scope of the present disclosure. Rather, it should be considered as exemplary of various embodiments that fall within the scope of the present disclosure as defined by the claims. Further, it should also be appreciated that references to “the disclosure” or “the present disclosure” should not be construed as meaning that the description is directed to only one embodiment or that every embodiment must contain a given feature described in connection with a particular embodiment or described in connection with the use of such phrases. In fact, various embodiments with common and differing features are described herein.

The present disclosure is generally directed to coatings that provide a noticeable improvement in anti-reflective properties. It is the combination of the improved anti-reflective properties with the anti-soiling properties, self-cleaning properties and manufacturing flexibility as well as other benefits that further enhances the utility of the coating. Accordingly, the coatings of the present disclosure may be used on substrates, such as transparent substrates, to increase the light transmittance through the substrates. In particular, the coatings may be used on transparent substrates such as glass or the front cover glass of solar modules.

Throughout this disclosure, solar modules are used as the exemplary embodiment, but it should be understood that any optical element may be utilized with the system and methods described herein, such as windows, architectural glass, LEDs, semi-conductors, exposed photovoltaic elements, lenses, diffusers, mirrors, windshields, automotive glass, screens/displays, goggles, eyeglasses, sunglasses, greenhouse glass, hybrid solar surfaces, marine glass, aviation glass, glass used in transportation, mobile device screens, and the like.

The present disclosure is particularly well suited for use with glass used in solar energy generation (“solar glass”). It should be understood that solar energy generation includes solar photovoltaic and solar thermal, wherein solar insolation is used to produce heat either as an end-point or as an intermediate step to generate electricity. Furthermore it should be understood that solar glass may be used in any application where maximal transmission of solar energy through the glass is desired such as for example in greenhouses or building environments where warm temperatures are desired. Typically solar glass is high transmission low iron glass. It may be either float glass, that is, flat glass sheets formed on a molten tin bath or patterned glass wherein the flat glass is formed by the action of rollers. Float glass is often characterized by the presence of tin contamination on the bottom (“tin side”) of the glass. Patterned glass is typically textured on one side to improve its performance in solar modules. It may also be formed into tubes such as those used as receivers in solar thermal energy generation or in some non-planar forms of solar photovoltaic generation. Embodiments of the present disclosure may also be applied to glass surfaces used as mirrors in solar energy generation such as parabolic trough systems or in heliostats. It may also be used to coat various glass lenses such as Fresnel lenses used in solar thermal generation.

Additionally, solar glass may have various coatings applied. For example a common coating is a transparent conductive oxide (TCO) such as fluorine doped tin oxide (FTO) or indium tin oxide (ITO) on one side of the glass. This coating is used to provide the front electrode for many thin film solar module technologies. Other coatings may be present such as coatings to seal in alkali ions such as Na+ and Ca+ that are used in the manufacturer of the glass but that cause long term reliability problems when leached out by water. Other techniques to solve this problem are to deplete these ions in thin layers of the glass surface. Solar glass may also be coated with a reflective surface to form a mirror. Solar glass may be tempered, annealed or untempered. Tempered glass is significantly stronger and solar modules manufactured using it typically only use one sheet of glass. If very thin tempered glass is used, then a second thin sheet of glass may be used as a back sheet for the solar module. Solar modules manufactured with untempered front glass typically use a back sheet of tempered glass to meet strength and safety requirements. Many thin-film solar photovoltaic technologies also use the front glass as a substrate upon which they deposit materials that comprise the solar cell. The processes used during the manufacturer of the solar cell may adversely affect the properties of any existing coatings on the glass or existing coatings may interfere with the solar cell manufacturing process. Embodiments of the present disclosure are completely tolerant of type of glass selected by the solar module manufacturer. It works well on float or patterned glass.

One critical issue for solar module manufacturers that use TCO (or similar) coated glass is tempering. It is very difficult to achieve low-cost, high quality TCO coated tempered glass. Therefore solar module manufacturers that requite TCO coated glass use untempered glass. Additionally even if suitable TCO coated tempered glass was available some thin-film solar manufacturing processes heat the glass during manufacturer to the extent that the temper is lost. Much of the anti-reflective glass on the market is tempered, because the anti-reflective coatings using the sol-gel process are actually formed during the tempering process. Tempering is the process by which the glass is heated to 600° C. to 700° C., then quickly cooled. This high tempering temperature sinters the anti-reflective coating providing it with its final mechanical strength. Thus solar module manufacturers that cannot use tempered glass typically cannot use anti-reflective glass. In addition, some module manufacturers, especially thin film module manufacturers who might need to apply anti-reflective coatings on finished or substantially finished modules are unable to use currently available sol-gel coatings because they need to be cured at temperatures greater than 300° C. or exposed to a corrosive ammonia atmosphere or exposed to highly toxic acids like hydrofluoric acid. Exposing finished or substantially finished solar modules to temperatures >300° C. or exposing them to a corrosive ammonia atmosphere is likely to damage their performance and/or long term reliability. Exposing finished modules to acids or other strong etchants to create a graded refractive index layer is equally challenging and poses an additional safety risk due to managing and disposing large quantities of a highly dangerous chemical like hydrofluoric acid. Embodiments of the disclosure may be applied and cured at a low temperature of between 20° C. and 300° C. and between 20° C. and 130° C. and further between 80° C. and 250° C. This low temperature facilitates the coating of completed solar panels without damage to the panel. Thus it is an anti-reflective solution for users of untempered solar glass and for users of anti-reflective coatings on finished or substantially finished solar modules.

The low temperature curing of Embodiments of the disclosure also provides substantial benefits to solar module manufacturers beyond enabling untempered anti-reflective glass. By making possible the coating of the glass without the need for the tempering step, solar module manufacturers are enabled to apply their own anti-reflective coating. Currently the requirement for a large tempering oven means that solar modules manufacturers are restricted to buying anti-reflective glass from glass manufacturers. This means that they must maintain inventory of both anti-reflective coated and non-coated glass. As these cannot be used interchangeably inventory flexibility is reduced necessitating keeping larger amounts of inventory on hand. The ability for the solar module manufacturer to apply their own coating means that they can just hold a smaller inventory of non-coated glass and then apply the anti-reflective coating to that as needed.

In addition, conventional anti-reflective coatings are prone to scratching during the solar module manufacturing process. Typically solar module manufacturers must use a plastic or paper sheet to protect the coating. As the coatings disclosed herein can be applied to fully manufactured solar modules, it can be applied at the end of the manufacturing process thus removing the need for the protection sheet and the opportunity for damage to the coating during manufacture.

Conventional anti-reflective coatings from different manufacturers tend to have subtle color, texture and optical differences. This presents problems to solar module manufacturers who desire their products to have a completely consistent cosmetic finish. If they manufacturer large numbers of solar modules it is almost inevitable that they will have to order anti-reflective glass from different suppliers causing slight differences in the appearance of the final products. However, the coatings disclosed herein enable solar module manufacturers to apply their own coating and so enables cosmetic consistency over an unlimited number of solar modules.

For anti-reflective coatings on solar modules, it would also be important to tune and optimize the thickness of the antireflective coating on glass depending upon the type of solar cell that is used by a solar module manufacturer. This is because the spectral responses for crystalline silicon, amorphous silicon, CdTe, CIGS, and other solar cell absorber materials have slight differences and it would be beneficial for the thickness of an antireflective coating to be optimized such that the maximum transmission for the antireflective coating occurs at wavelengths that are well matched to that of the underlying solar cell material.

In addition, to their anti-reflective properties, the coatings described herein can exhibit anti-soiling and/or self-cleaning properties, as they are resistant to the adhesion of dirt and promote the removal of any adhered dirt by the action of water. More specifically, some embodiments of the coatings described herein can be characterized by extremely fine porosity that minimizes the deposition of dirt by physical means. Further, some embodiments of the coatings are characterized by a low energy surface that resists chemical and physical interactions and makes it easy to dislodge the particles, thereby making the surfaces essentially anti-soiling. The reduced physical and/or chemical interactions with the environment, such as dirt, make the exposed surface of these coating less susceptible to binding of dirt and also make it easier to clean with a minimal expenditure of force or energy.

Typically in order to completely clean ordinary glass a mechanical action for example using brushes or high-pressure jets is required to dislodge dirt that is strongly adhered to the surface. However some embodiments of the coatings described herein present a surface such that dirt is more attracted to water then to the surface. Thus in the presence of water any dirt resting on the surface is efficiently removed without the need for mechanical action. This means that coated glass may achieve a high level of cleanliness in the presence of natural or artificial rain without human or mechanical intervention. In addition, the amount of water required to clean the glass is reduced. This is of special significance given that the most effective locations for solar energy generation tend to be sunny warm and arid. Thus water is a particularly expensive and scarce resource in the very locations that solar energy is most effective.

Embodiments of the disclosure enable a reduction in the Levelized Cost of Energy (LCOE) to the operator of a solar energy generating system. First, the anti-reflective property increases the efficiency of the solar modules. Increased efficiency enables a reduction of cost in the Balance of System (BOS) costs in construction of the solar energy generation system. Thus for a given size of system the capital costs and construction labor costs are lower. Second, the anti-soiling property increases the energy output of the solar modules by reducing the losses due to soiling. Third the Operating and Maintenance (O&M) costs are reduced because fewer or no washings are needed reducing labor and water cost associated with washing.

In some embodiments described herein the coating can also contain water and oil resistant hydro/fluoro-carbon groups that make them chemically less reactive and less interacting. When used in combination with a glass substrate, the coatings bind to the glass surface using siloxane linkages that make them adhere strongly and makes them strong, durable, and abrasion and scratch resistant. These coatings are physically and chemically less reactive, mechanically and structurally stable, hydrophobic, oleophobic, stable, resistant to degradation by solar ultra-violet (UV) light. Accordingly, it should be appreciated that the coatings described herein have particular application to transparent substrates that are exposed to the environment, such as exterior windows and glass used by solar modules.

Generally, the coatings described herein are prepared by a sol-gel process. The starting composition, referred to as a “coating mixture” or “coating composition,” includes a hydrolysate of organotrialkoxysilane or a combination of organoalkoxysilanes and tetraalkoxysilane in a form of a homogenous gel-free solution of sol. This sol can be coated onto a substrate using coating techniques known in the art, dried to form a gel, and cured to form a hard layer or coating having the properties noted above. The process of curing the dried gel further densifies the coating.

Generally, the resulting properties of the coating described above are provided by using a particular combination of components in the formation of the final coating. In particular, the selection of a particular organoalkoxysilane precursor or mixture of organoalkoxysilane precursors in combination with other components in the coating mixture is important in providing a coating with the desired properties. For example, in some embodiments, the coatings are made from a mixture of organoalkoxysilane precursors including tetraalkoxysilane, organofunctionalalkoxysilane, and fluorine-containing organoalkoxysilanes. In some embodiments, separate coating mixtures or mixtures of organofunctionalalkoxysilane precursors can be used to form separate sols that may then be combined to form a final sol that is applied to a substrate to be coated. Further, a single sol, or separately prepared sols that are combined together, may be combined with another organofunctionalalkoxysilane precursor to form a final sol that is applied to a substrate to be coated.

For example, tetraalkoxysilanes when hydrolyzed form an extensively cross-linked structure due to the formation of four Si—O—Si linkages around each silicon atom. These structures are characterized by mechanical stability and abrasion resistance. To impart hydrophobicity and anti-soiling to the ultimate coating, organoalkoxysilanes (such as methyltrimethoxysilane) can be used in addition to the tetraalkoxysilane. Further, to impart oleophobicity and anti-soiling characteristics, fluorine-containing organoalkoxysilanescan be used in addition to the tetralkoxysilane.

It should be appreciated that the coating material and process by which it is applied to the substrate can comprise a larger coating system. The coating material is optimized for a particular coating method and vice versa. Thus the optimized coating process is performed by a tool optimized to insure consistency and quality. Therefore this tool coupled with the coating materials comprises the coating system. Given that the benefits of the current disclosure are particularly well suited to solar module manufacturers, who do not themselves manufacture tools; it is desirable to offer a complete solution consisting of the coating material, the coating process knowledge and the associated coating tool. In the following paragraphs describing the coating process it should be appreciated that these steps could be executed manually, automatically using a coating tool or in any combination of both. It should also be appreciated that there are several possible coating systems wherein different coating materials, coating processes and coating tools are used in combination.

The tool may be a large stand-alone unit intended for operation in a factory setting; it could be a sub-tool, such that it comprises a process module that performs the coating process but that is integrated into another machine that performs other steps in the larger solar module manufacturing process. For example it could be a module attached to an existing glass washing machine or a module attached to a solar module assembly machine. Alternatively, the tool could be portable or semi-portable for example mounted on a truck or inside a tractor trailer such that it could be transported to a worksite and used to coat solar modules during the construction of a large solar installation. Alternatively it could be designed such that the coating could be applied to installed solar modules in situ.

In general, three steps are used to apply the sol to a given substrate. First, the substrate is cleaned and pretreated. Second, the substrate is coated with the sol or mixture of sols. Third, the final coating is formed on the substrate.

As an initial step, the substrate is pretreated or pre-cleaned to remove surface impurities and to activate the surface by generating a fresh surface or new binding sites on the surface.

It is desirable to increase the surface energy of the substrate through pretreatment or cleaning of the substrate surface to form an “activated” surface. For example an activated surface may be one with many exposed Si—OH moieties. An activated surface reduces the contact angle the sol and enables effective wetting of the sol on the surface. In some embodiments, a combination of physical polishing or cleaning and/or chemical etching is sufficient to provide even wetting of the sol. In cases, where the surface tension would need to be further lowered, the substrate, such as glass, may be pretreated with a dilute surfactant solution (low molecular weight surfactants such as surfynol; long chain alcohols such as hexanol or octanol; low molecular weight ethylene oxide or propylene oxide; or a commercial dishwasher detergent such as CASCADE, FINISH, or ELECTRASOL to further help the sol spread better on the glass surface.

Accordingly, surface pretreatment can involve a combination of chemical and physical treatment of the surface. The chemical treatment steps can include (1) cleaning the surface with a solvent or combination of solvents, detergents, mild bases like sodium carbonate or ammonium carbonate and/or (2) cleaning the surface with a solvent along with an abrasive pad, (3) optionally chemically etching the surface, and/or (4) washing the surface with water. The physical treatment steps can include (1) cleaning the surface with a solvent or combination of solvents, (2) cleaning the surface with a solvent along with particulate abrasives, and (3) washing the surface with water. It should be appreciated that a substrate can be pretreated by using only the chemical treatment steps or only the physical treatment steps. Alternatively, both chemical and physical treatment steps could be used in any combination. It should be further appreciated that the physical cleaning action of friction between a cleaning brush or pad and the surface is an important aspect of the surface preparation.

In the first chemical treatment step, the surface is treated with a solvent or combination of solvents with variable hydrophobicity. Typical solvents used are water, ethanol, isopropanol, acetone, and methyl ethyl ketone. A commercial glass cleaner (e.g., WINDEX) can also be employed for this purposes. The surface may be treated with an individual solvent separately or by using a mixture of solvents. In the second step, an abrasive pad (e.g., SCOTCHBRITE) is rubbed over the surface with the use of a solvent, noting that this may be performed in conjunction with the first step or separately after the first step. In the last step, the surface is washed or rinsed with water.

One example of substrate preparation by this method involves cleaning the surface with an organic solvent such as ethanol, isopropanol, or acetone to remove organic surface impurities, dirt, dust, and/or grease (with or without an abrasive pad) followed by cleaning the surface with water. Another example involves cleaning the surface with methyl ethyl ketone (with or without an abrasive pad) followed by washing the surface with water. Another example is based on using a 1:1 mixture of ethanol and acetone to remove organic impurities followed by washing the surface with water.

In some instances an additional, optional step of chemically etching the surface by means of concentrated nitric acid, sulfuric acid, or piranha solution (1:1 mixture of 96% sulfuric acid and 30% H₂O₂) may be necessary to make the surface suitable for bonding to the deposited sol. Typically this step would be performed prior the last step of rinsing the surface with water. In one embodiment, the substrate may be placed in piranha solution for 20 minutes followed by soaking in deionized water for 5 minutes. The substrate may then be transferred to another container holding fresh deionized water and soaked for another 5 minutes. Finally, the substrate is rinsed with deionized water and air-dried.

The substrate may alternatively or additionally prepared by physical treatment. In the physical treatment case, for one embodiment the surface is simply cleaned with a solvent and the mechanical action of a cleaning brush or pad, optionally a surfactant or detergent can be added to the solvent, after which the substrate is rinsed with water and air dried. In another embodiment the surface is first cleaned with water followed by addition of powdered abrasive particles such as ceria, titania, zirconia, alumina, aluminum silicate, silica, magnesium hydroxide, aluminum hydroxide particles, silicon carbide, or combinations thereof onto the surface of the substrate to form a slurry or paste on the surface. The abrasive media can be in the form a powder or it can be in the form of slurry, dispersion, suspension, emulsion, or paste. The particle size of the abrasives can vary from 0.1 to 10 microns and in some embodiments from 1 to 5 microns. The substrate may be polished with the abrasive slurry via rubbing with a pad (e.g., a SCOTCHBRITE pad), a cloth, a foam, or paper pad. Alternatively, the substrate may be polished by placement on the rotating disc of a polisher followed by application of abrasive slurry on the surface and rubbing with a pad as the substrate rotates on the disc. Another alternative method involves use of an electric polisher that can be used as a rubbing pad in combination with abrasive slurry to polish the surface. The substrates polished with the slurry are cleaned by water and air-dried.

After pretreating the surface, the coating is deposited on a substrate by techniques known in the art, including dip coating, spray, drop rolling, flow coating or roll coating to form a uniform coating on the substrate. Other methods for deposition that can be used include spin-coating; aerosol deposition; ultrasound, heat, or electrical deposition means; micro-deposition techniques such as ink-jet, spay-jet, xerography; or commercial printing techniques such as silk printing, dot matrix printing, etc. Deposition of the sol is may be done under ambient conditions or under controlled temperature and humidity conditions. In some embodiments the temperature is controlled between 20° C. and 35° C. and/or the relative humidity is controlled between 20% and 60% or more preferably between 25% and 35%.

In some embodiments, the method of deposition is performed via the drop rolling method on small surfaces wherein the sol composition is placed onto the surface of a substrate followed by tilting the substrate to enable the liquid to roll across the entire surface. For larger surfaces, the sol may be deposited by flow coating wherein the sol is dispensed from a single nozzle onto a moving substrate at a rate such that the flowing sol leads to a uniform deposition onto a surface or from multiple nozzles onto a stationary surface or from a slot onto a stationary surface. Flow coating is described in greater detail elsewhere herein. Another method of deposition is via depositing the liquid sol onto a substrate followed by use of a mechanical dispersant to spread the liquid evenly onto a substrate. For example, a squeegee or other mechanical device having a sharp, well-defined, uniform edge may be used to spread the sol such as roll coating which is described in greater detail herein.

In addition to the actual methods or techniques used to deposit the final sol on the substrate, several variations for depositing the final sol exist. For example, in some embodiments, the final sol is simply deposited on the substrate in one layer. In other embodiments, a single sol or multiple sols may be deposited to form multiple layers, thereby ultimately forming a multilayered coating. For example, a coating of the sol containing at least one high silanol terminal group can be formed as an underlayer for better adhesion to glass substrate followed by a topcoat of a tetraalkoxysilane to obtain a better abrasion resistant outer surface. In another embodiment, an underlayer of an organosilane may be deposited followed by the deposition of a topcoat of a mixture of an organofluoroalkoxysilane and a tetraalkoxysilane. In another embodiment, an underlayer of a tetraalkoxysilane may be deposited followed by the deposition of a top layer using a sol mixture of an organoalkoxysilane and an organofluoroalkoxysilane. In another embodiment, an underlayer of a sol made from a mixture of an organoalkoxysilane and an organofluoroalkoxysilane may be deposited followed by vapor deposition of a top layer by exposing the layer to vapors of a tetraalkoxysilane. In another embodiment, an underlayer of a sol made from a mixture of an organoalkoxysilane and an organofluoroalkoxysilane may be deposited followed by deposition of a top layer by immersing the substrate in a solution of a tetraalkoxysilane in isopropanol. In the embodiments in which multiple layers are deposited, each layer may be deposited shortly after deposition of the first layer, for example, within or after 30 seconds of deposition of the prior layer. As noted, different sols may be deposited on top of one another, or different mixtures of sols may be deposited on top of one another. Alternatively, a single sol may be deposited in multiple layers or the same sol mixture may be deposited in multiple layers. Further, a given sol may be deposited as one layer and a different sol mixture may be used as another layers. Further, any combination of sols may be deposited in any order, thereby constructing a variety of multi-layered coatings. Further, it should be appreciated that the sols for each layer may be deposited using different techniques if so desired.

The thickness of the coatings deposited can vary from about 10 nm to about 5 μm. In some embodiments, the thickness of the coating varies from about 100 nm to about 1 micron, and in other embodiments it varies from about 100 nm to about 500 nm. In order to provide sufficient anti-reflective properties, a thickness of about 60 nm to about 150 nm is desired. The thickness of the coating mixture as deposited is affected by the coating method, as well as by the viscosity of the coating mixture. Accordingly, the coating method should be selected so that the desired coating thickness is achieved for any given coating mixture. Further, in those embodiments, in which multiple layers of sols are deposited, each layer should be deposited in a thickness such that the total thickness of the coating is appropriate to achieve the final desired transmission spectrum properties. Accordingly, in some embodiments in which multiple layers of sols are deposited, the overall coating thickness varies from about 100 nm to about 500 nm, and in order to provide sufficient anti-reflective properties, a total coating thickness of about 60 nm to about 150 nm is desired.

Once the final sol is deposited as described above, the deposited sol will dry to form a gel through the process of gelation after which the gel is cured to remove residual solvent and facilitate further condensation of Si—OH and network formation via Si—O—Si linkage formation in the coating. In addition, the gel may be allowed to age to allow for the formation of additional linkages through continued-condensation reactions.

As described above, the sol-gel method used in preparing the coatings described herein utilizes a suitable molecular precursor that is hydrolyzed to generate a solid-state polymeric oxide network. Initial hydrolysis and partial condensation of the precursor monomers generates a liquid sol, which ultimately turns to a solid gel during drying. Drying of the gels under ambient conditions (or at elevated temperature) leads to evaporation of the solvent phase to form a cross-linked film. Accordingly, throughout the process, the coating mixture/sol/gel/dried/cured coating undergoes changes in physical, chemical, and structural parameters that intrinsically alter the material properties of the final coating. In general, the changes throughout the sol-gel transformation can be loosely divided into three interdependent aspects of physical, chemical, and structural changes that result in altered structural composition, morphology, and microstructure. The chemical composition, physical state, and overall molecular structure of the sol and the gel are significantly different such that the materials in the two states are intrinsically distinct.

Regarding physical differences, the sol is a collection of dispersed silanol-containing polysilsesquioxane oligomers (low Mw polymer) dissolved in a solvent. These silanol-containing polysilsesquioxane oligomers are surrounded by a solvent shell and do not interact with each other significantly. As such, the sol is characterized by fluidity and exists in a liquid state. In contrast, in a gel film the network formation has occurred to an advanced state such the particles are interconnected to each other. The increased network formation and cross-linking makes the gel network rigid with a characteristic solid state. The ability of the material to exist in two different states is because of the chemical changes (condensation of Si—OH) that occur along the sol to gel transformation.

Regarding chemical changes, during the sol to gel transition, the sol macro-molecules combine with each other via formation of Si—O—Si linkages. As a result, the material exhibits network formation and strengthening. Overall, the sol macro-molecules contain reactive Si—OH silanol groups that can participate in network formation while the gel structure has these Si—OH silanol groups converted into siloxane groups.

Regarding structural differences, the sol contains few siloxane linkages along with terminal Si—OH silanol as well as unhydrolyzed alkoxy ligands. As such, the sol state can be considered structurally different from the solidified films, which contain majority siloxanes. As such, the liquid sol and the solid state polymeric networks are chemically and structurally distinct systems.

Some combination of organoalkoxysilane precursors could provide a sol with a long shelf-life, while some combination of organoalkoxysilane precursors could provide a sol that could gel into a coating with superior abrasion resistance, while some other combination of organoalkoxysilane precursors could gel into a coating with abrasion resistance and anti-soiling properties. Regarding differences in properties, the origin of the physical and chemical properties of the sol and gel films depends upon their structure. The sol particles and the gel films differ in the chemical composition, makeup and functional groups and as a result exhibit different physical and chemical properties. The sol stage because of its particulate nature is characterized by high reactivity to form the network while the gel state is largely unreactive due to conversion of reactive Si—OH silanol groups to stable siloxane linkages. Accordingly, it is the particular combination of organoalkoxysilane precursors and other chemicals added to the coating mixture that is hydrolyzed and condensed, gelled, dried and cured on a substrate surface that gives the final coatings of the present disclosure the desired properties described above.

There are several methods by which the gel is dried and cured and/or aged to form the final coating. In some embodiments the gel is dried and cured under ambient or room temperature conditions. In some embodiments, the gel is aged under ambient conditions for 30 minutes followed by drying for 3 hours in an oven kept at a variable relative humidity of (e.g., 20% to 50%). The temperature of the oven is then increased slowly at a rate of 5° C./min to a final temperature of 120° C. The slow heating rate along with the moisture slows the rate of the silanol condensation reaction to provide a more uniform and mechanically stable coating. This method provides reproducible results and is a reliable method of making the coating with the desired properties.

In another embodiment, the gel on the substrate is heated under an infrared lamp or array of lamps. These lamps are placed close proximity to the substrate's coated surface such that the surface is evenly illuminated. The lamps are chosen for maximum emission in the mid-infrared region of 3˜5 μm wavelength. This region is desirable because it is adsorbed better by glass than shorter infrared wavelengths. The power output of the lamps may be closely controlled via a closed loop PID controller to achieve a precise and controllable temperature profile. In some embodiments this profile will start from ambient temperature and quickly rise 1° C. to 50° C. per second to a temperature of 120° C., hold that temperature for a period of 30 to 300 seconds, then reduce temperature back to ambient, with or without the aid of cooling airflow.

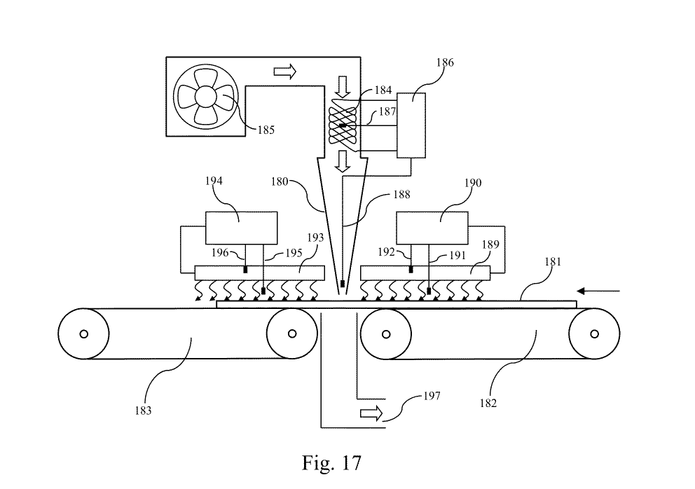

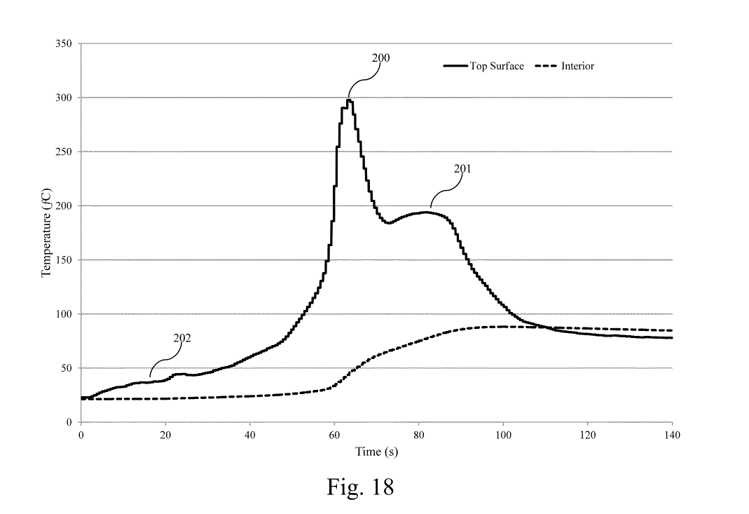

For applications requiring high throughput and/or for applications wherein there is a process sensitivity around the maximum allowable temperature for the bottom surface of the coated glass when the glass is cured it would be preferred to cure the glass such that only the top surface of the glass is heated by impinging hot air on the coated surface or a xenon arc lamp using a pulsing method where the lamp is turned on and off multiple times during the cure cycle. One curing technique known as skin curing is described in greater detail elsewhere herein with reference to FIG. 17.

It is particularly noteworthy that the coatings of this disclosure can be prepared under temperatures not exceeding 120° C. in contrast to temperatures of 400° C. to 600° C. typically employed in curing silica-based anti-reflective coatings.

As described above and as illustrated further in the examples, the coatings made as described herein have several desirable properties. The coatings have anti-reflective properties that reduce the reflection of photons. The transmittance of a glass substrate coated with a coating composition made according to the present disclosure can be increased by about 1% to about 8%, from about 2% to about 6%, and from about 1% to about 4% relative to uncoated glass substrates

The coatings also have anti-soiling properties, which are also important in maintaining sufficient transmittance when used in conjunction with a glass substrate. Soiling is due to adherence of particulate matter on surfaces exposed to environment. The deposition of the particles onto surfaces depends upon the surface microstructure as well as chemical composition. In general, rough surfaces can provide many sites for physical binding of particulate matter.

The chemical composition of the surfaces is reflected in the surface energy as measured by contact angles. Low energy surfaces (characterized by high water contact angles) are usually less susceptible to binding as compared to high energy surfaces with low water contact angles. Therefore, anti-soiling properties can be determined indirectly by measuring the coating's contact angle. The coatings herein provide contact angles ranging from about 10 degrees to about 178 degrees, from about 110 degrees to about 155 degrees, and from about 125 degrees to about 175 degrees. The coatings of this disclosure minimize the photon flux losses due to soiling by about 50% relative to uncoated samples.

The coatings of the present disclosure also provide tunable mechanical properties. Nanoindentation is a method of used to measure the mechanical properties of nanoscale materials especially thin films and coatings. The testing instrument that is used for performing the nanoindentation tests is a Nanomechanical Test System (manufactured by Hysitron, Inc., USA). This Nanomechanical Test System is a high-resolution nanomechanical test instrument that performs nano-scale quasi-static indentation by applying a force to an indenter tip while measuring tip displacement into the specimen. During indentation, the applied load and tip displacement are continuously controlled and/or measured, creating a load-displacement curve for each indent. From the load-displacement curve, nano-hardness and reduced elastic modulus values can be determined by applying the Oliver and Pharr method and a pre-calibrated indenter tip area function and a pre-determined machine compliance value. The instrument can also provide in-situ SPM (scanning probe microscopy) images of the specimen before and after indentation. Such nanometer resolution imaging function is accomplished quickly and easily by utilizing the same tip for imaging as for indentation. The in-situ SPM imaging capability is not only useful in observing surface features, but also critical in positioning the indenter probe over such features for indentation tests.

Typically nanohardness and reduced elastic modulus will be determined using nanoindentation. The reduced elastic modulus has a relationship with the Young's modulus as shown in Equation 1. If Poisson's ratio for the material to be tested is known then Young's modulus of it can be calculated. The Poisson's ratio for the diamond indenter is 0.07 and the Young's modulus of the indenter is 1141 GPa.

$$\frac{1}{{E}_{r}} = \frac{( 1 - {v}_{material}^{2} )}{{E}_{material}} + \frac{( 1 - {v}_{indenter}^{2} )}{{E}_{indenter}} \qquad Eq . 1$$

The nanoindentation tests were performed on 1 cm² samples cut from coated glass specimens made according to composition of Example 2 and Example 3. To obtain the hardness and modulus values for the coating, ten indents were performed on each sample. Loads of 15 μN were used for Sample 5F and 25 μN for Sample 7J. All indents were performed through in-situ SPM imaging. Table 1 summarizes the test conditions and parameters used in the nanohardness and modulus tests.

TABLE 1
Nanohardness and Modulus Testing Conditions and Parameters

| | |
|---|---|
| Specimens | Sample 5F and Sample 7J |
| Test instrument | TriboIndenter |
| Indentation Load | 15, 25 μN |
| Indenter Probe Tip | Diamond Berkovich indenter tip |
| Temperature | 74° F. |
| Humidity | 25% RH |
| Environment | Ambient air |

Tables 2 and 3 present the nanohardness, H, and reduced elastic modulus, Er, measurement results. These tables also show values for the contact depth, hc, of each indent. The test locations of these indents were chosen to ensure adequate spacing between measurements.

From Tables 2 and 3, it can be known that the average nanohardness was highest for Sample 7J (2.11 GPa) and lowest for Sample 5F (1.43 GPa). Average reduced elastic modulus was highest for Sample 7J (20.99 GPa) lowest for Sample 5F (13.51 GPa). These results further confirm that the hardness of the coatings of the disclosure can be tuned by changing the ratios of organoalkoxysilane, tetraalkoxysilane and organofluoroalkoxysilanes in the synthesis of sols from which the coatings are obtained.

TABLE 2
Nanohardness and Reduced Elastic Modulus Test Results
for Sample 5F - Film Made from Composition of Example 2

| Test Under | H | Er | hc |
|---|---|---|---|
| 15 μN | (GPa) | (GPa) | (nm) |
| 1 | 1.46 | 13.93 | 15.24 |
| 2 | 1.45 | 13.67 | 15.16 |
| 3 | 1.48 | 13.38 | 14.98 |
| 4 | 1.46 | 13.21 | 15.13 |
| 5 | 1.48 | 13.37 | 15.02 |
| 6 | 1.34 | 13.50 | 16.04 |
| 7 | 1.46 | 13.55 | 15.23 |
| 8 | 1.43 | 13.95 | 15.40 |
| 9 | 1.43 | 13.57 | 15.41 |
| 10 | 1.34 | 13.00 | 16.06 |
| Average | 1.43 | 13.51 | 15.37 |
| St. Dev | 0.05 | 0.30 | 0.39 |

TABLE 3
Nanohardness and Reduced Elastic Modulus Test Results
for Film 7J - Film Made from Composition of Example 3

| Test Under | H | Er | hc |
|---|---|---|---|
| 25 μN | (GPa) | (GPa) | (nm) |
| 1 | 2.09 | 20.76 | 15.87 |
| 2 | 2.04 | 20.75 | 16.10 |
| 3 | 2.09 | 20.53 | 15.72 |
| 4 | 2.27 | 21.75 | 14.99 |
| 5 | 2.08 | 21.15 | 15.82 |
| 6 | 2.13 | 21.40 | 15.59 |
| 7 | 2.09 | 20.78 | 15.80 |
| 8 | 2.03 | 21.20 | 16.09 |
| 9 | 2.15 | 21.22 | 15.59 |
| 10 | 2.11 | 20.30 | 15.78 |
| Average | 2.11 | 20.99 | 15.73 |
| St. Dev | 0.07 | 0.44 | 0.31 |

The coatings of the present disclosure also provide desirable abrasion resistance. Abrasion resistance can be defined as the ability of a material to withstand erosion due to frictional forces to preserve and maintain its original shape and appearance. Abrasion resistance relates to the strength of the intrinsic framework structure as well as to surface features. Materials that do not have sufficient strength due to lack of long range bonding interactions tend to abrade easily. Similarly, materials with uneven surfaces or coatings with surface inhomogeneities and asperities tend to wear due to frictional losses. Also, the leveling and smoothening of these asperities due to friction leads to changes in optical transmission of the coating as the material is abraded.

The coatings of the present disclosure pass the standard test for measuring abrasion resistance of coatings on surfaces as defined according to European Standard EN1096.2 (Glass in Building, Coated Glass). The test involves the action of rubbing a felt pad on the coated glass. The felt rubbing pad is subjected to a to-and-fro translation motion with a stroke length of 120±5 mm at a speed of 54-66 strokes/min combined with a continuous rotation of the pad of 6 rpm or of a rotation of between 10° to 30° at the end of each stroke. The back and forth motion along with the rotation constitutes 1 cycle. The specifications of the circular felt rubbing pad include a diameter of 14-15 mm, thickness of 10 mm and density of 0.52 g/cm². The felt pad is attached to a mechanical finger that is 15 mm to 20 mm is diameter and placed under a load of 4 Newtons. The transmission between 380 nm and 1100 nm is measured to evaluate abrasion resistance and the standard dictates an absolute change in transmission of no more that 0.5% with respect to a reference sample.

TABLE 4
Varying of Abrasion Resistance by Changing The
Ratio of Precursors on Tin-Sided TCO Glass

| | Pre-Abrasion | Post-Abrasion |
|---|---|---|
| Composition | Transmission Gain | Transmission Gain |
| Example 2 | 2.56 | 1.69 |
| Example 3 | 3.17 | 2.83 |
| Example7 | 2.49 | 2.49 |
| Example 8 | 2.08 | 1.83 |
| Example 9 | 2.69 | 2.43 |
| Example 10 | 2.06 | 1.95 |

The coatings of the present disclosure have abrasion resistance that can be tuned or modulated in a variety of ways. Examples in Table 4 demonstrates how the abrasion resistance of the coatings from this disclosure can be tuned or modulated by changing sol composition from which the coatings are obtained. It would be beneficial to be able to provide coatings as in Example 3 that have a higher durability against abrasion for solar modules or glass substrates that are exposed to abrasive natural environments like sandstorms or cleaning actions that involve contacting the antireflective coatings with abrasives. In areas where the solar modules are unlikely to be exposed to significant abrasive environments it might be more beneficial to provide coatings that have a higher pre-abrasion transmission as in Example 2.

It is possible that the beneficial properties of the coating can also be tuned by changing the molecular weight of the sols that comprise the coating or changing the ratio of low and high molecular weight components in the sols that comprise the coating or by the changing the polydispersity of the sols that comprise the coating. Altering the pH and changing the catalysts used in the reaction could also be used to change the molecular weights and molecular weight distribution or polydispersity of the components in the sol. For example, changing the polydispersity of the sols could impact how the polymerized silane molecules pack together. This could have an impact on abrasion resistance of the cured coating. Another example is modifying the surface characteristics of the final coating by the presence of low molecular weight hydrolyzed organofluoroalkoxysilane molecules in the sol. As the coating dries, these low molecular weight species could rise to the coatings surface and modify the wettability of the coating and thereby alter its anti-soiling and/or self-cleaning properties.

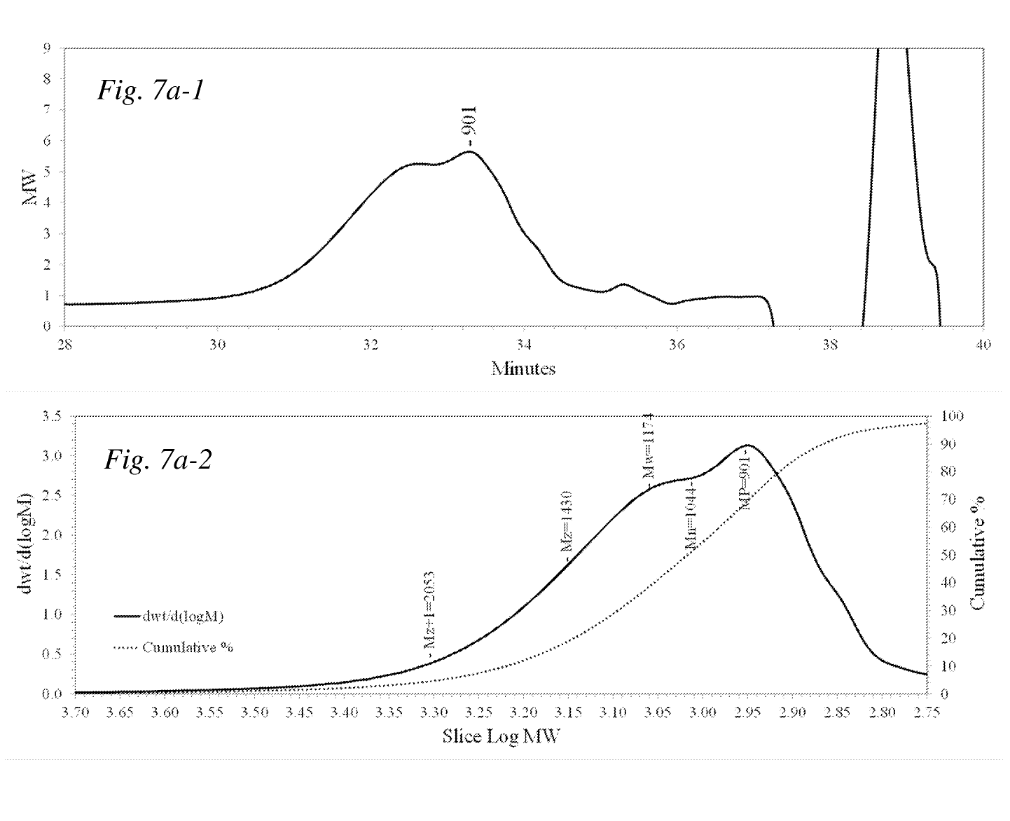

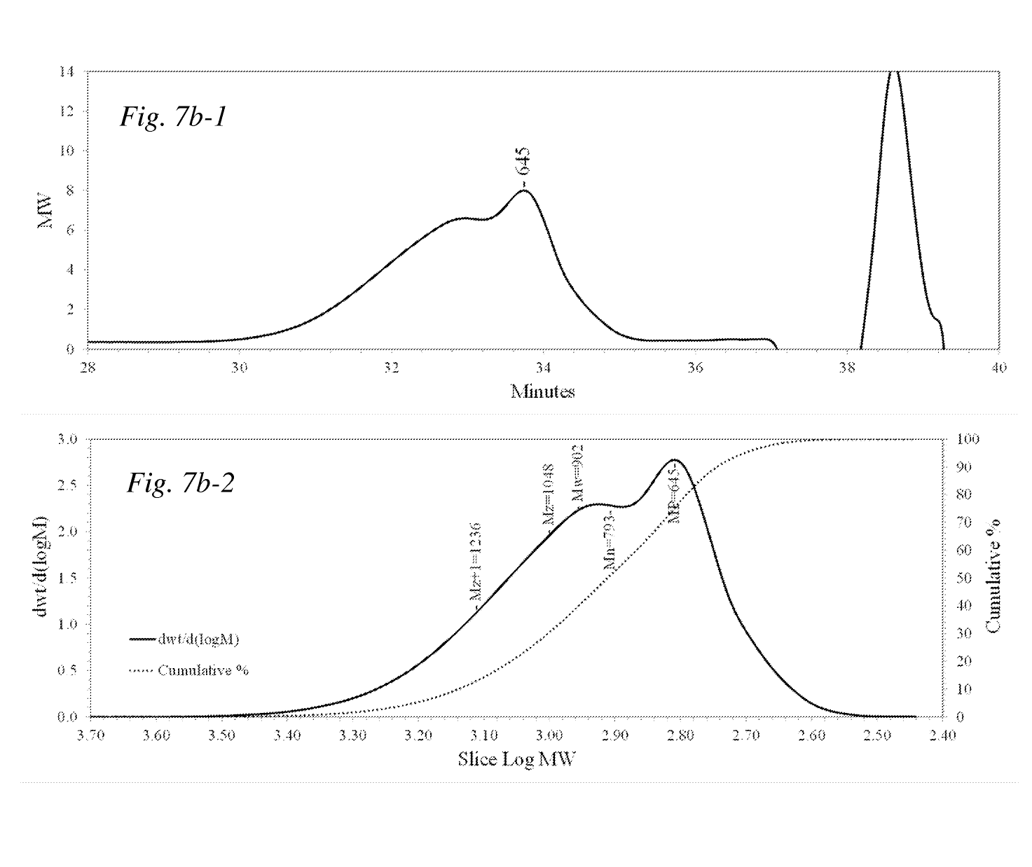

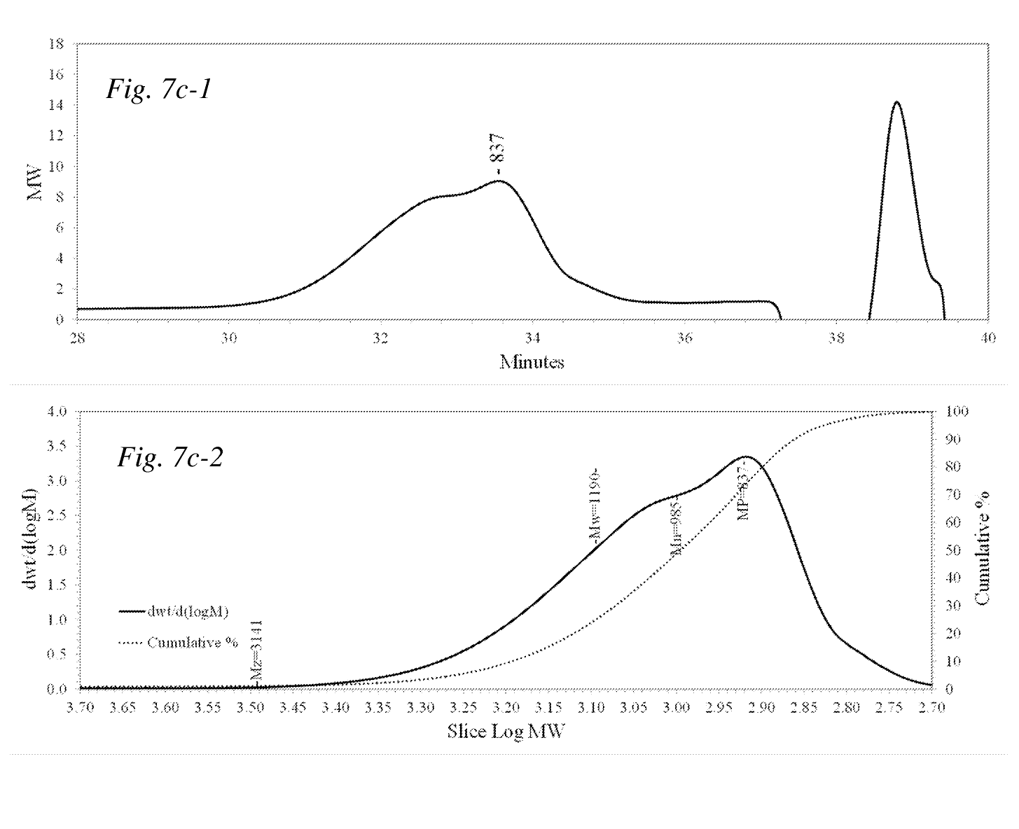

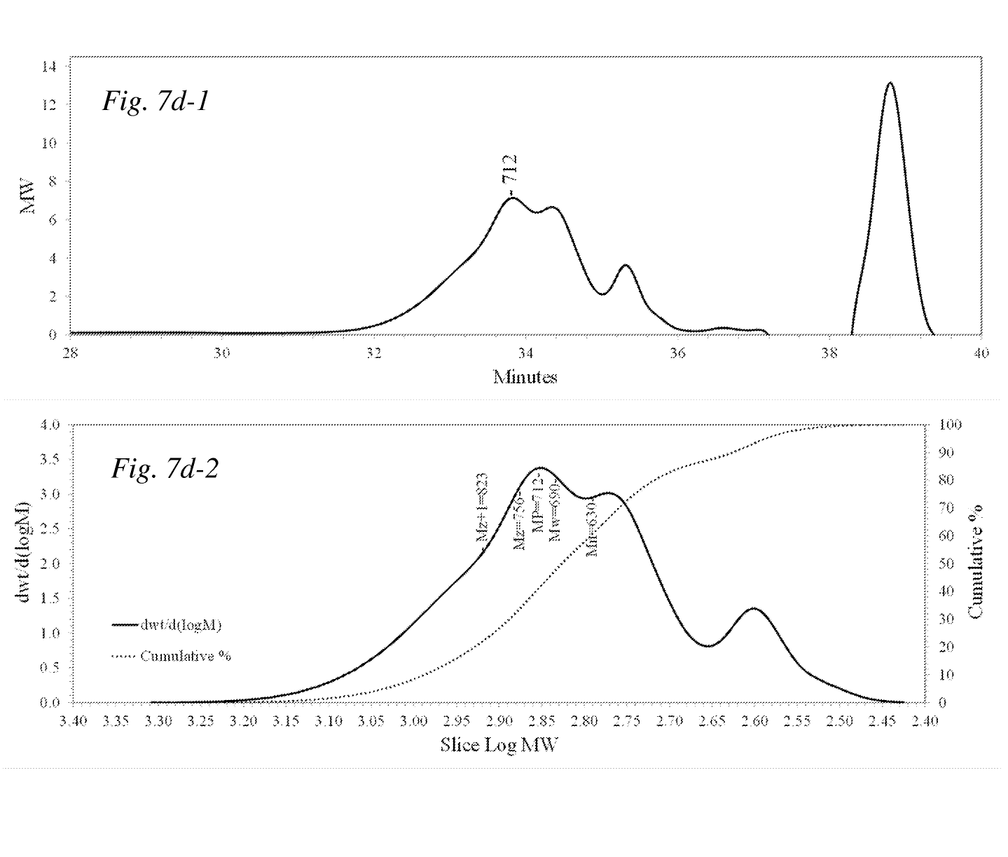

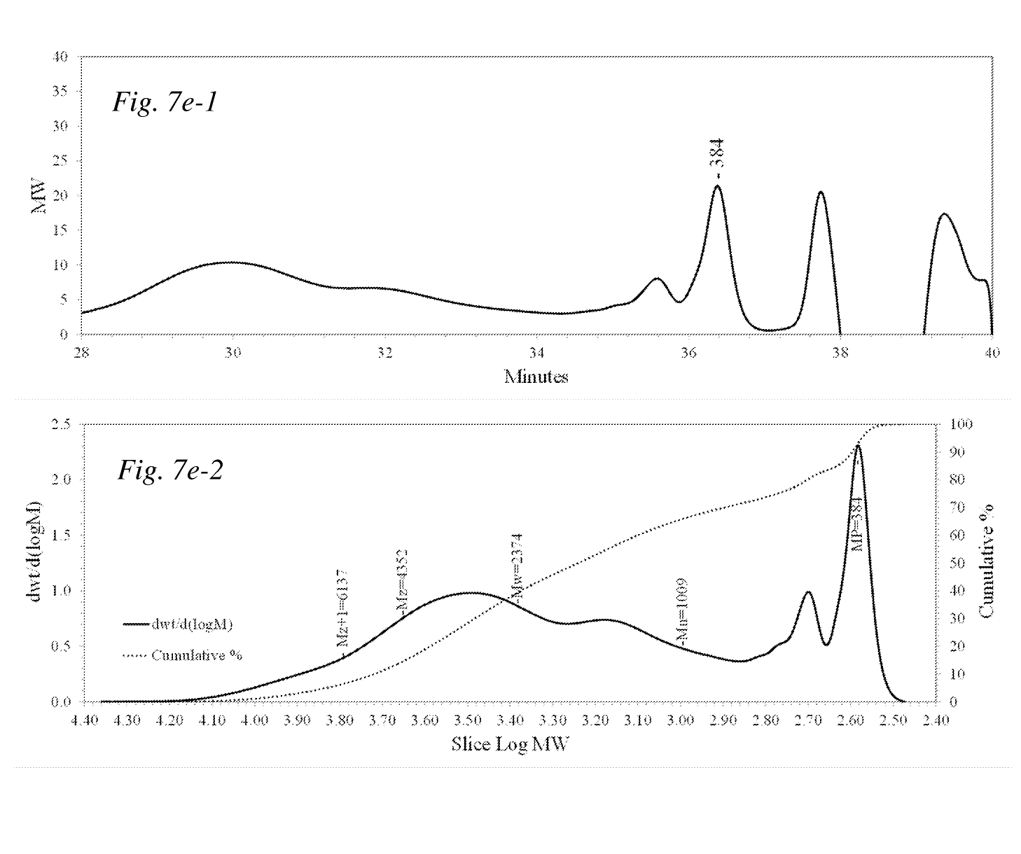

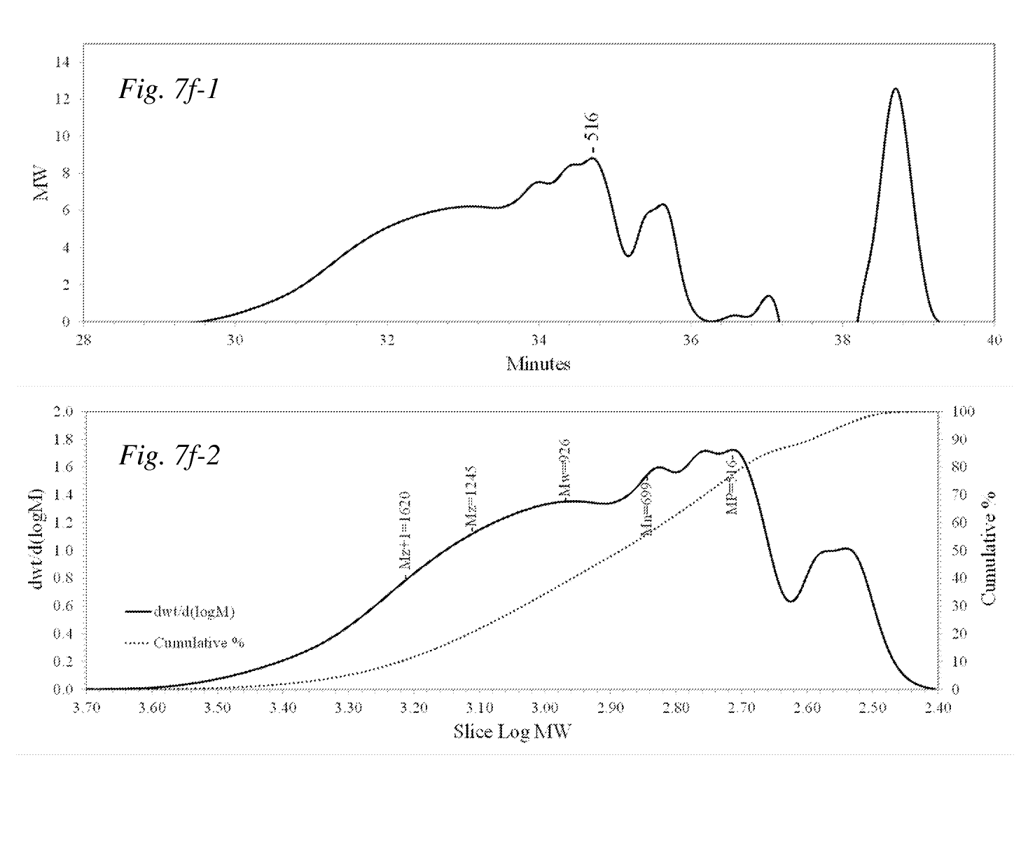

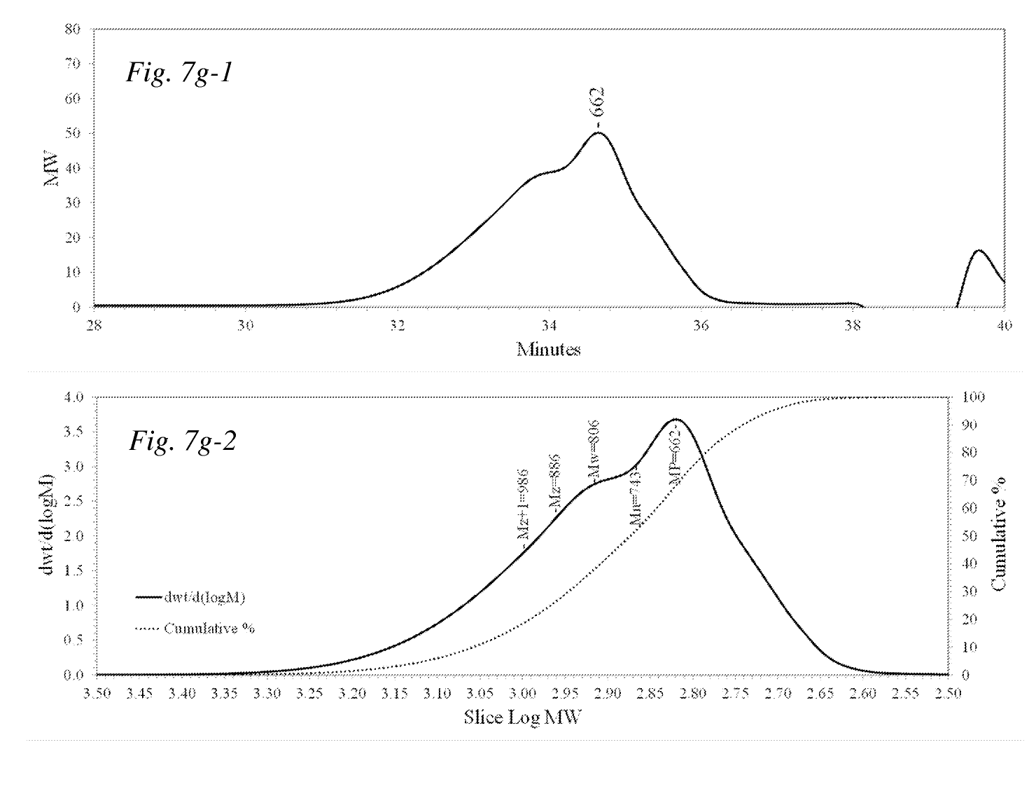

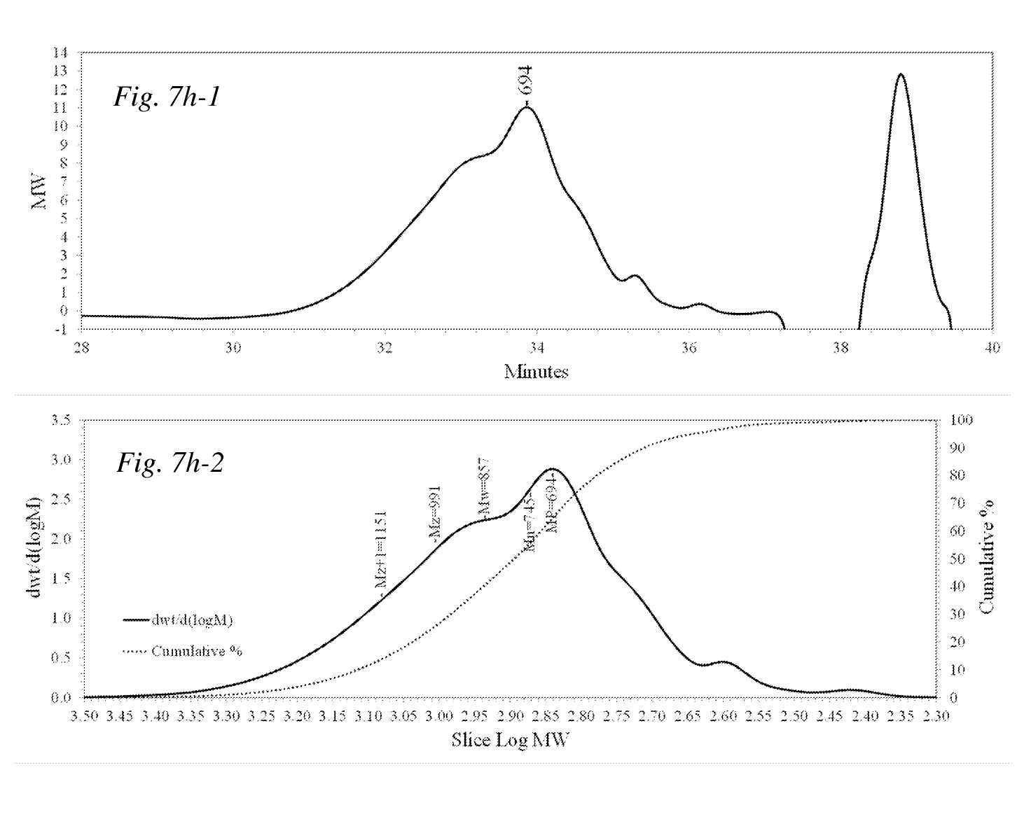

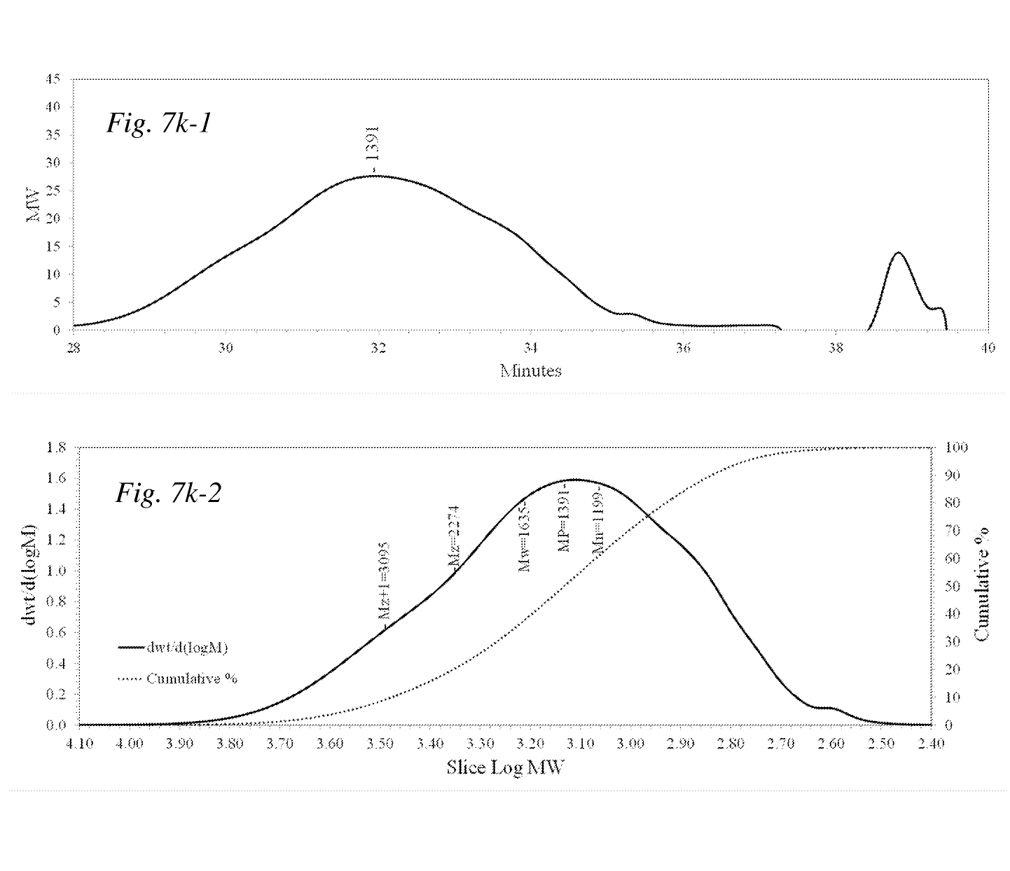

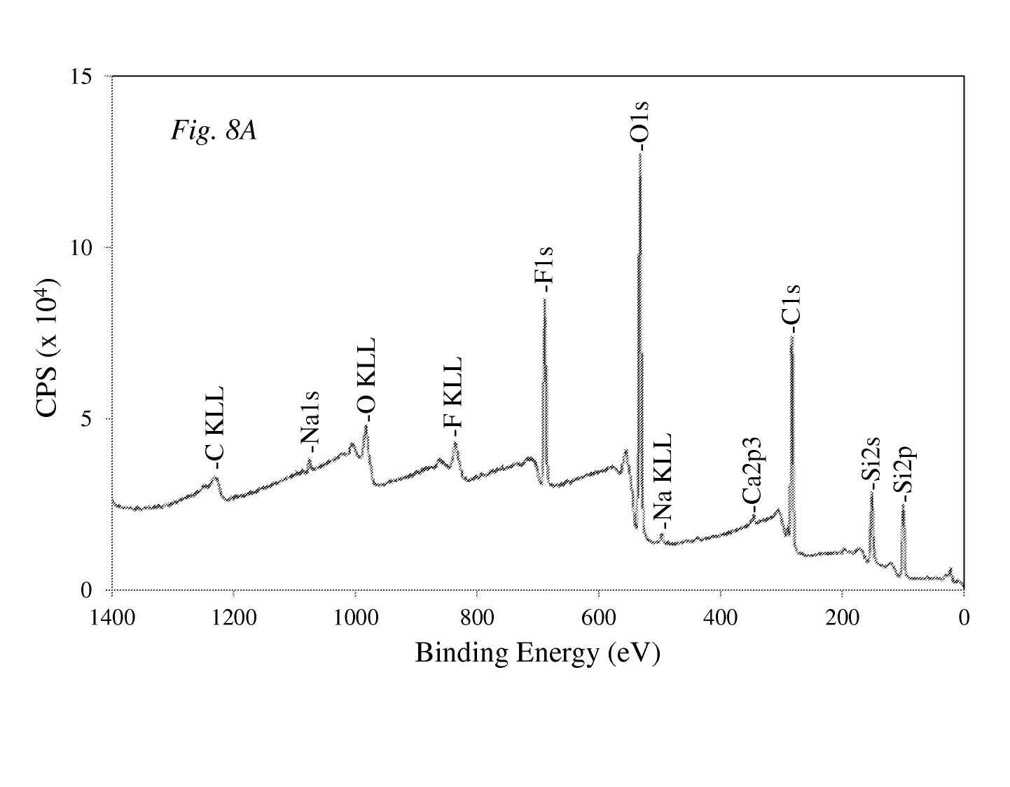

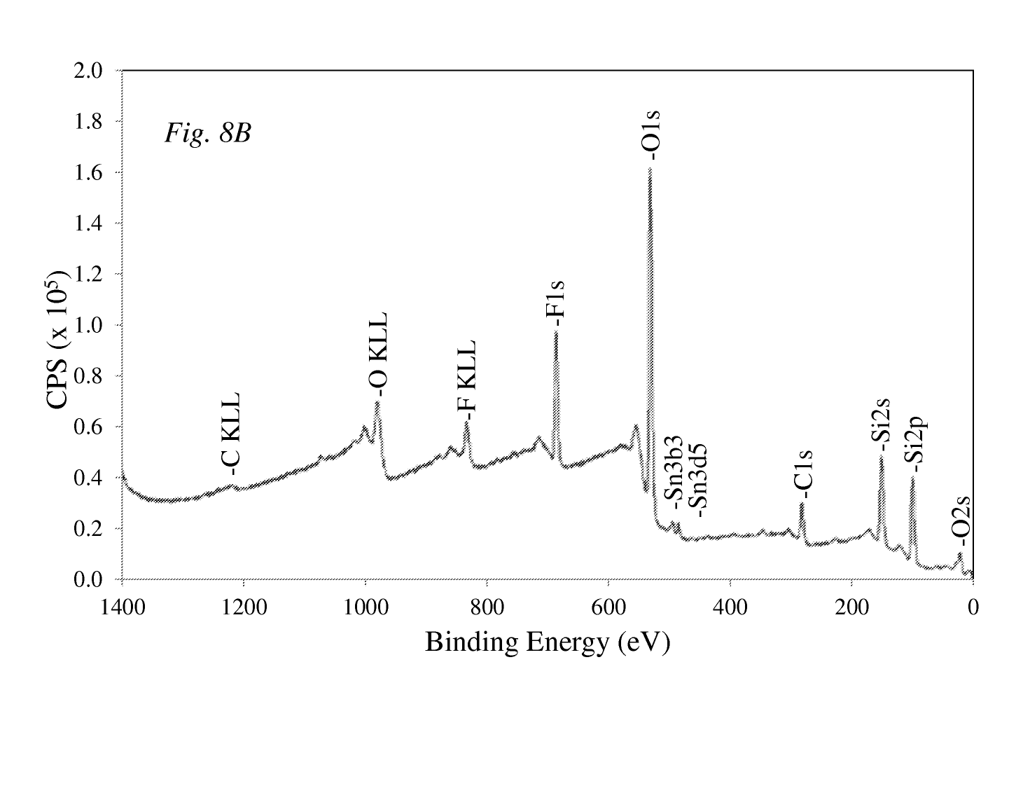

Gel Permeation Chromatography (GPC) is a technique that is used to characterize the molecular weight of polymers. We have used Waters GPC systems for molecular weight analysis. The method details are as follows HPLC system: a 1515 isocratic pump equipped with 2707 Autosamplerand 50 uL loop, 2414 RI detector with column heater. Column and detector oven heated to 40° C. Flow used was 1.0 ml/min. Four 4.6×300 mm GPC columns in-line: lus Styragel HR 2 for an effective MW range were used. The columns came pre-equilibrated in THF and THF was used as the eluent. Polystyrene narrow standards were used and the standard curve was fit to a 3rd-order polynomial. Nine polystyrene standards from approx. 530 MW to 50,000 MW were used. The PS standards were prepared at 10 mg/ml each in THF and diluted their samples 1:10 in THF for injections. Results are shown in FIG. 7a and FIG. 7b for sol made from Example 2. It can be seen that the sols used for preparation of coatings in this disclosure can have a weight average Molecular Weight (Mw) of less than 1000 and number average molecular weight of (Mn) of less than 1000 with polydispersity (PD) of less than 1.2.

Yet another way to modulate the abrasion resistance of the coatings of the present disclosure is by changing the temperature at which the coatings are cured after drying. Similar films when cured at ambient temperature typically will have a lower abrasion resistance compared to films cured at 120° C. which can be lower than films cured at 200° C. or 300° C. in a conventional oven.

In general, the various coatings of the present disclosure provide a means of making a transparent substrate or glass transmit more photons without altering its intrinsic structure and other properties, along with passivating the surface so that it becomes resistant to the adhesion of water, dirt, soil, and other exogenous matter. Accordingly, the coating mixtures and resulting gels and coatings as described herein have numerous commercial applications.

Regarding the coating mixtures themselves, these may be packaged for commercial sale as a coating mixture or commercial coating formulation for others to use. For example, the coating mixtures may be provided as a liquid composition, for example, for subsequent small scale treatment of glass in a treatment separate from their usage as windows in solar or architectural systems. In this case the coating mixture may be packaged for sale as sols after the silane precursors have been hydrolyzed.

One or more of the following organic solvents can be used for formulation of sol in coating mixture; methanol, ethanol, 1-propanol, 2-propanol, 1-butanol, ethyl acetate, propylene glycolmethyether, propyleneglycol, propyleneglycolmethyletheracetate, acetone, cyclohexanone, methylethylkeone, dimethylether, diethylether, 2-butanol, 2-butanone, tetrahydrofurane, 1,2-diethoxyethane, diethyleneglycol, 1,2 dimethoxymethane and methyl t-butyl ether,

In addition, the coating mixtures may be deposited and allowed to gel on a particular substrate that is subsequently packaged for sale as a finished assembly. In particular, the coating compositions of the present disclosure can be coated onto any transparent substrate that has hydrogen bond donor or hydrogen bond acceptor groups on the surface. For example, the coating can be applied as a treatment for a given glass or other transparent substrate before or after it has been integrated into a device, such a solar cell, optical window or enclosure, for example, as part of a glass treatment process. In other embodiments, the disclosure provides for the use of the coating compositions as an efficiency enhancement aid in architectural windows in building and houses by the provision of anti-reflection benefits and/or by the provision of anti-soiling benefits to augment the anti-reflection benefits. In other embodiments, the disclosure provides for the use of the coating compositions as an efficiency enhancement aid in treatment of transparent surfaces that require regular cleaning to make them self-cleaning. For example, the coatings can be used in conjunction with glass used in windows, windshields, screens, architecture, goggles, eyeglasses, etc.

In other embodiments, the disclosure provides for the use of the coating compositions as an efficiency enhancement aid in photovoltaic solar module assemblies (e.g., the outer cover of solar modules) by the provision of anti-reflection benefits and/or by the provision of anti-soiling benefits to augment the anti-reflection benefits. These devices convert solar energy into electrical energy and rely upon efficient absorption of photons, and effects such as reflection, scattering, and loss of absorption due to adsorbed soil or dirt particles can lead to reduced power output. As noted, the coatings of this disclosure when coated onto a glass surface reduces reflection of photons (the so-called anti-reflective property) and also reduces adsorption and binding of dirt, soil, and other particulate matter from the environment to boost the transmission of photons through the glass as well as to prevent reduction in photons associated with deposition of particulate matter onto the surface.

The coatings for solar module applications provide unique challenges that are not present with coatings typically utilized in other common applications. The use of anti-reflective coating in solar modules necessitates long-term exposure of solar radiation that usually results in extensive degradation of polymeric materials under prolonged UV exposure due to photolytic breakdown of bonds in these materials. The coating compositions of the present disclosure utilize silane precursors that when hydrolyzed and dried and cured give rise to a network that is similar to glass with Si—O—Si bonds that are stable to radiative breakdown. An additional advantage of using silica based materials in solar applications is the intrinsic hardness of the material that makes the coating resistant to scratches, indentations, and abrasion. Further, the coatings of the present disclosure provide for enhanced light transmittance across the entire solar region from about 350 nm to about 1150 nm, which is desirable for solar applications.

Further, the sols resulting from the coating compositions of this disclosure do not need to be applied to the solar modules during manufacturing and may be applied after manufacturing to avoid any interference with the solar module manufacturing process. It is expected that the solar module maker themselves may be able to use the composition of this disclosure to coat the modules at appropriate points within their manufacturing process. In such instances, the provision of a stable sol, that can be used according to the methods described herein, provides a direct means for the applying the coating mixture after manufacture of the modules or even after final installation of the modules. This may streamline the manufacturing process and enhance the economic value of existing modules, either existing inventory or modules already installed and in use, to which the coatings can be applied.

In one embodiment, the process of coating the solar modules consists of preparing the module surface, coating the surface with the final sol made in accordance with the present disclosure, drying the coating under ambient conditions, and curing the dried modules at elevated temperature. The module surface is prepared by polishing the module with a cerium oxide slurry, followed by washing the module with water, and drying it under ambient temperature-pressure conditions for a period ranging from about 10 hours to about 12 hours.

Once the module surface is prepared, in one embodiment, the final sol of the present disclosure is deposited onto solar modules by means of a flow coater. The sol is deposited onto the modules via gravitational free flow of the liquid sol from top to bottom. The solar modules are placed on the mobile platform that moves at a rate that is optimal for the free flow of the sol without introducing break points in the liquid stream or introducing turbulent flow. The rate of liquid flow and the rate of movement of platform carrying the solar module are optimized for deposition of uniform, crack-free coatings that are homogenous, free of deformities, and characterized by uniform thickness.

More specifically, in one embodiment, the module is placed on a mobile stage that is connected to a computer and programmed to move at a speed ranging, in some embodiments, from about 0.05 cm/s to about 300 cm/s, in other embodiments from about 0.1 cm/s to about 10 cm/s, and in other embodiments from about 0.25 cm/s to about 0.5 cm/s. The sol is then deposited onto the module surface using a computer controlled nozzle dispensing unit such that the rate of flow of sol is, in some embodiments, from about 5 ml/min to about 50 ml/min, in other embodiments from about 5 ml/min to about 25 ml/min, and in other embodiments from about 10 ml/min to about 15 ml/min. The rate at which the sol is deposited is important for proper deposition of the coatings. Notably, the nozzle diameter of the sol can be adjusted to ensure appropriate flow rate, with diameters of the nozzle ranging from about 0.3 mm to about 0.9 mm.

A particularly advantageous aspect of using a sol is that it is in a liquid state but is also viscous enough to spread without breakdown of the stream. The uniformity of the coatings is further ensured by adjusting the flow rate and the rate of the movement of the platform containing the solar modules. For a given flow rate of the sol, if the rate of the movement of the platform is too fast then it leads to rupture of the sol stream causing uneven coatings. For a given flow rate of the sol, if the rate of the movement of the platform is too slow it results in excessive flow and material build up that deteriorates the uniformity of the films. Therefore, a specific optimum of sol flow rate and the platform movement are important to provide even, uniform, and homogenous coatings. The use of specific pH, solvent, and silane concentrations as outlined above provide the ideal viscosities.

The coating process is also facilitated by the evaporation of the solvent during the flow of the sol onto the panel, which also affects the development of uniform films or coatings on the module surface. The coatings are formed when the free flowing sol dries on the surface and forms a solid on the glass surface. More specifically, the bottom edge of the sol represents the leading wet line while drying occurs at the top edge. As the solvent evaporates, the sol becomes more viscous and finally sets at the top edge while the bottom edge is characterized by liquid edge spreading. The spreading liquid at the bottom edge enables the free flow of the sol while the setting sol at the top edge fixes the materials and prevents formation of lamellar structures. A balance of these factors is important for formation of uniform films.

The flow coating method does not allow seepage of the sol into the internal parts of the solar module assembly as the excess sol can be collected into a container at the bottom of the assembly and recycled. Similarly, it does not facilitate corrosion and/or leaching of the chemicals from the interior of the solar module assembly. The flow coater method exposes only the glass side to the sol while the other side of the module assembly, which may contain with electrical contacts and leads does not come into any contact with the liquid sol. As such, the flow coating process is particular beneficial to coating solar modules during either the assembly or the post-assembly stages.

The methods described here can be used to coat solar modules of variable sizes and in variable configurations. For example, typical modules have the dimensions of about 1 m×1.6 m, which can be coated either in portrait configuration or landscape configurations mode via appropriate placement in the mobile platform.

The flow coater can be used to coat the modules at the rate of about 15-60 modules per hour. The rate of coating of individual modules would depend upon the size of the modules and whether they are coated in the portrait mode or landscape orientation. Additionally, multiple coaters operating in parallel, or a single coater that runs along the entire length can be used in conjunction with the module assembly line to increase the production rate.

After depositing the coating, the module is dried for a period ranging from about 1 minute to about 20 minutes or longer under ambient conditions. The coated module is then cured using any of the techniques for curing described above, after which the coated module is ready for use.

The anti-reflecting coatings described herein increase the peak power of the solar cells by approximately 3% due to the anti-reflective property. In addition, it is estimated that the anti-soiling property would contribute to minimize transmissive losses associated with accumulation of dirt on the modules. Typical soiling losses are estimated at about 5% and use of these coatings is expected to reduce the losses in half.

### Examples

The following describes various aspects of the coatings made according to certain embodiments of the disclosure in connection with the Figures. These examples should not be viewed as limiting. The general procedure used for the preparation of sol from hydrolysis of methyltrialkoxysilane and tetraalkoxysilane and fluoropropyltrialkoxysilane is described as follows:

In the 1^{st} part of the sol, a 500 ml flask was charged with DIW, HCl and IPA stir @ 100 rpm at RT for short time (1 min) followed by addition of methyltrialkoxysilane. The reaction was stirred at ˜100 rpm at RT for 30 min. Similarly 2^{nd} part of the sol was prepared by charging a 500 ml flask with DIW, HCl and IPA stir @ 100 rpm for short time (˜1 min) followed by addition of trifluoropropyltrialkoxysilane. The 2^{nd} part was stirred at ˜100 rpm at RT for 30 min. The 1^{st} and 2^{nd} parts of the sol were combined and tetraalkoxysilane was added to this mixture and stirred for another 30 minutes at ˜100 rpm at RT. The final sol product mixture was aged for 48 hours at RT and then characterized by GPC and NMR.

In one embodiment referred to as Example 1, Sol I was prepared by charging a 500 mL flask with 177 g of IPA and 25 g of 0.04 M HCl. After stirring @ 100 rpm at RT for short time (˜1 min), 2.87 g (0.021 moles) of methyltrimethoxysilane (MTMOS) was added to the mixture. The mixture was stirred at RT for 30 min. Sol II was prepared by charging a 500 mL flask with 177 g of IPA and 25 g of 0.04 M HCl. After stirring @ 100 rpm at RT for short time (˜1 min), 3.71 g (0.021 moles) of (3,3,3-trifluoropropyl)-trimethoxysilane (F3TMOS) was added to the mixture. The mixture was stirred at RT for 30 min. Sol I and II were mixed together followed by addition of 6.39 g (0.042 moles) of tetramethoxysilane (TMOS). The final mixture was stirred at RT for 30 min. This mixture was allowed to age under ambient conditions for 24 hours up to 120 hours. After aging sol formulation, a 30×30 cm glass sheets (polished with cerium oxide polish, washed, and allowed to dry) were flow coated with the final sol mixture and allowed to dry for approximately 1-10 minutes followed by curing at temperature of 120° C. for 60 minutes.

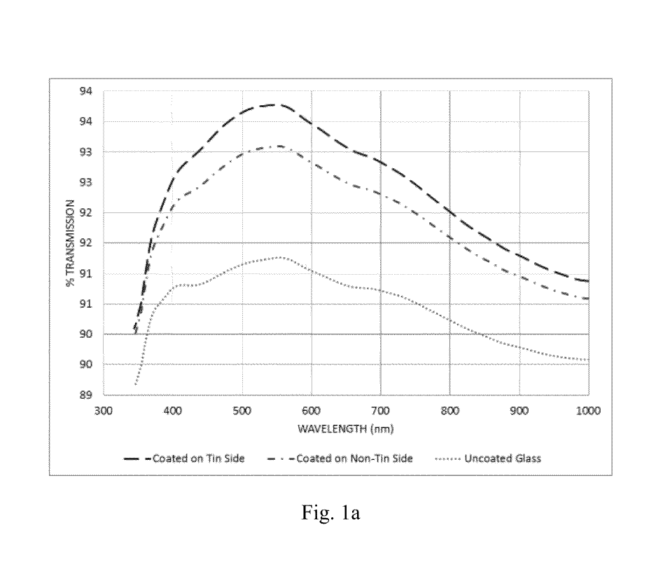

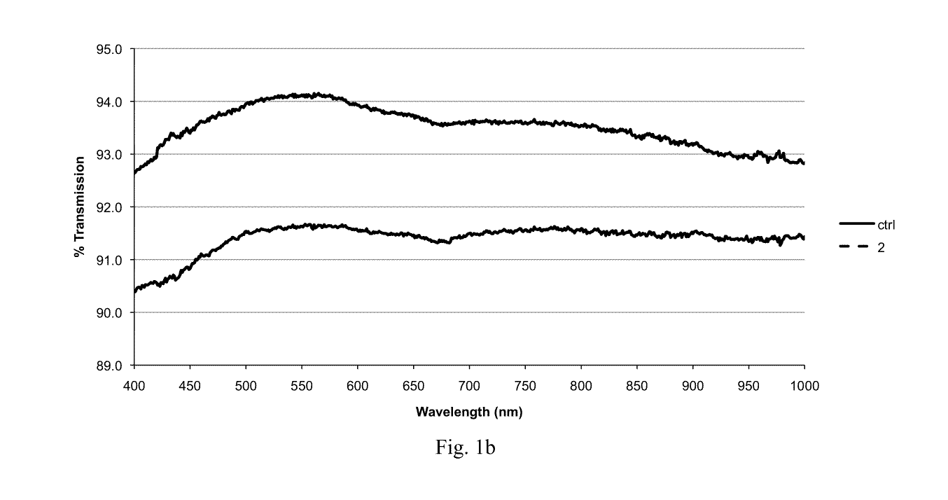

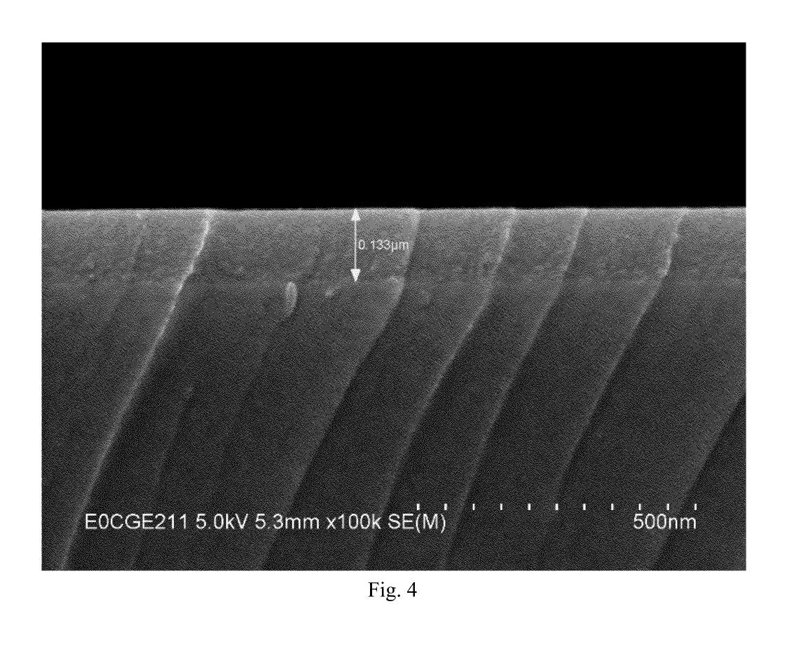

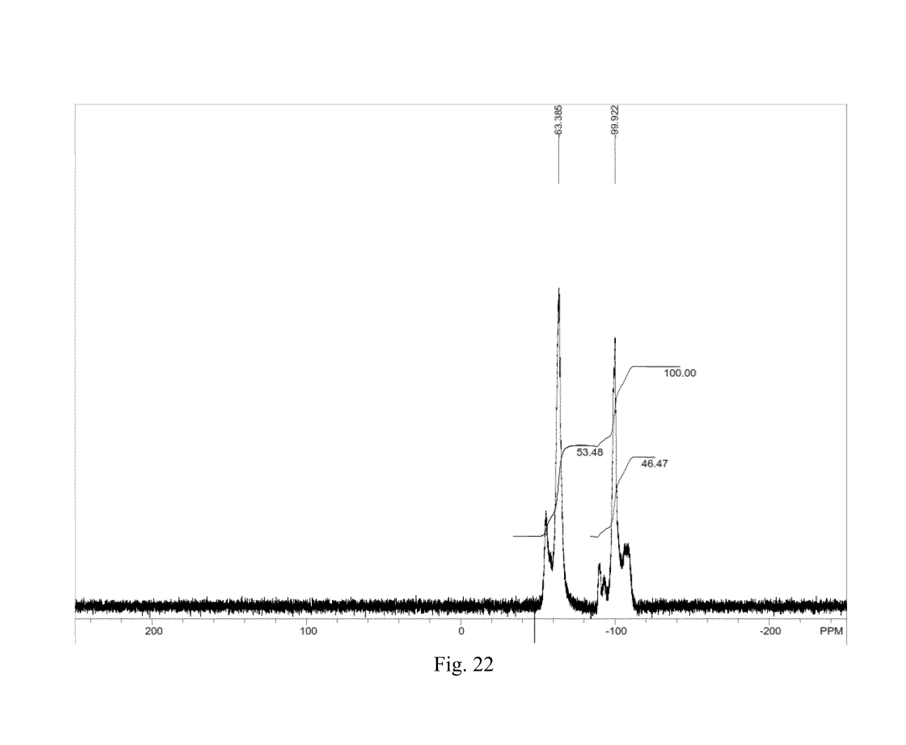

In another embodiment referred to as Example 2, Sol I was prepared by charging a 500 mL flask with 177 g of IPA and 25 g of 0.04 M HCl. After stirring @ 100 rpm at RT for short time (˜1 min), 2.87 g (0.021 moles) of methyltrimethoxysilane (MTMOS) was added to the mixture. The mixture was stirred at RT for 30 min. Sol II was prepared by charging a 500 mL flask with 177 g of IPA and 25 g of 0.04 M HCl. After stirring @ 100 rpm at RT for short time (˜1 min), 3.71 g (0.021 moles) of (3,3,3-trifluoropropyl)-trimethoxysilane (F3TMOS) was added to the mixture. The mixture was stirred at RT for 30 min. Sol I and II were mixed together followed by addition of 8.8 g (0.042 moles) of tetraethoxysilane (TEOS). The final mixture was stirred at RT for 30 min. This mixture was allowed to age under ambient conditions for 48 hours. The GPC test of the final sol produced molecular weight of Mw=1,137 g/mol with polydispersity PD=1.13. After aging sol formulation, a 30×30 cm glass sheets (polished with cerium oxide polish, washed, and allowed to dry) were flow coated with the final sol mixture and allowed to dry for approximately 1-10 minutes followed by curing at temperature of 120° C. for 60 minutes. The GPC results are shown in FIGS. 7a-1 and 7a-2 and the Si-NMR results are shown in FIG. 21 and FIG. 22. SEM cross-section of a representative sample of cured film from example 2 is shown in FIG. 4

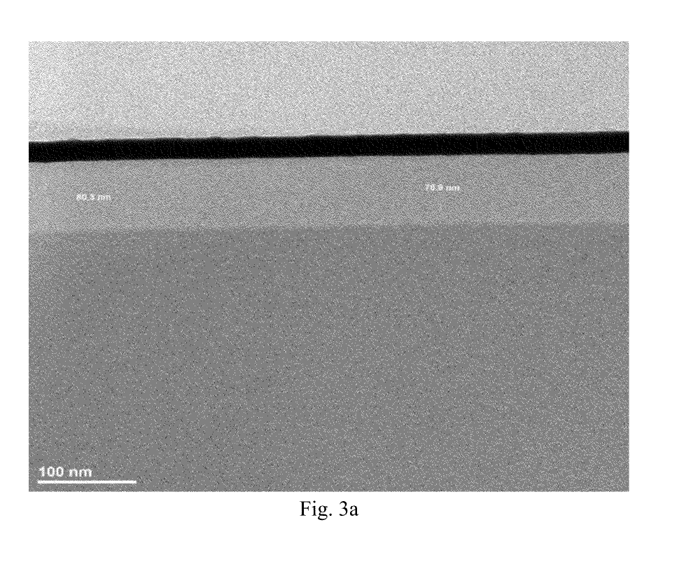

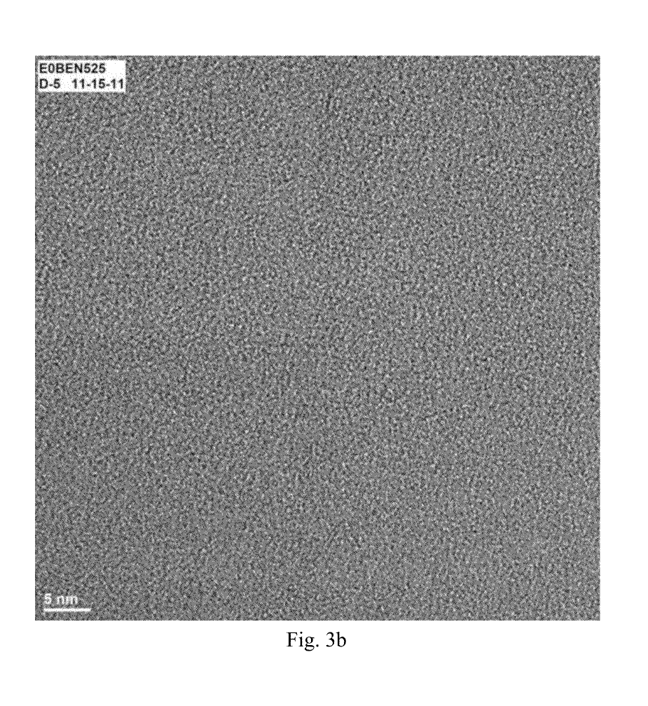

TEM cross-section of a representative sample of the dried and cured film from example 2 is shown in FIG. 3a. TEM cross-section and the High Resolution TEM of the film from example 2 show no evidence of long range order within the film. The film morphology at a scale of 5 nm show little evidence of porosity.

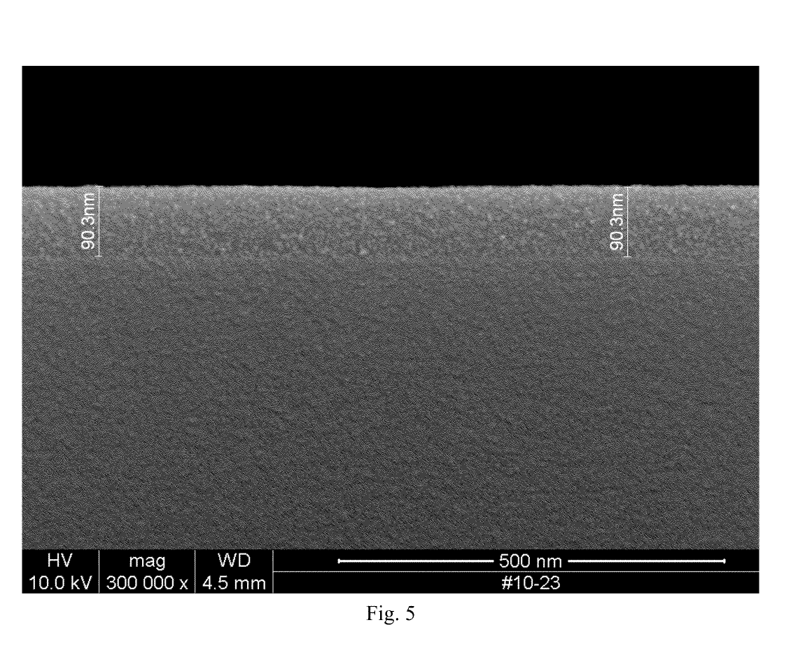

In yet another embodiment referred to as Example 3, sol was prepared by charging a 500 mL flask with 354 g of IPA and 50 g of 0.04 M HCl. After stirring @ 100 rpm at RT for a short time (˜1 min), 4.37 g (0.0321 moles) of methyltrimethoxysilane (MTMOS) was added to the mixture. The mixture was stirred at RT for 30 min followed by addition of 7.08 g (0.034 moles) of tetraethoxysilane (TEOS). The final mixture was stirred at RT for 30 min. This mixture was allowed to age under ambient conditions for 48 hours. The GPC test of the final sol produced molecular weight of Mw=906 g/mol with polydispersity PD=1.10. A 30×30 cm glass sheets (polished with cerium oxide polish, washed, and allowed to dry) were flow coated with the final sol mixture and allowed to dry for approximately 1-10 minutes followed by curing at temperature of 120° C. for 60 minutes. GPC results are shown in FIGS. 7b-1 and 7b-2, and NMR results are shown in FIG. 21 and FIG. 22. SEM cross-section of a representative sample of cured film from example 3 is shown in FIG. 5.

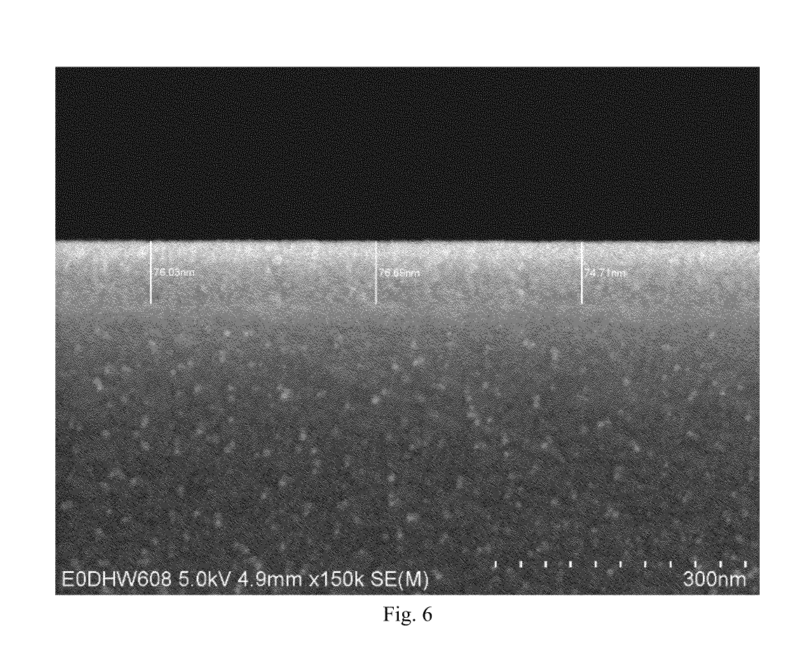

In yet another embodiment referred to as Example 4, Sol I was prepared by charging a 500 mL flask with 177 g of IPA and 25 g of 0.04 M HCl. After stirring @ 100 rpm at RT for short time (˜1 min), 2.63 g (0.0193 moles) of methyltrimethoxysilane (MTMOS) was added to the mixture. The mixture was stirred at RT for 30 min. Sol II was prepared by charging a 500 mL flask with 177 g of IPA and 25 g of 0.04 M HCl. After stirring @ 100 rpm at RT for short time (˜1 min), 0.284 g (0.0013 moles) of (3,3,3-trifluoropropyl)-trimethoxysilane (F3TMOS) was added to the mixture. The mixture was stirred at RT for 30 min. Sol I and II were mixed together followed by addition of 6.24 g (0.03 moles) of tetraethoxysilane (TEOS). The final mixture was stirred at RT for 30 min. This mixture was allowed to age under ambient conditions for 48 hours. The GPC test of the final sol produced molecular weight of Mw=690 g/mol with polydispersity PD=1.10. A 30×30 cm glass sheets (polished with cerium oxide polish, washed, and allowed to dry) were flow coated with the final sol mixture and allowed to dry for approximately 1-10 minutes followed by curing at temperature of 120° C. for 60 minutes. GPC results are shown in FIGS. 7c-1 and 7c-2. SEM cross-section of a representative sample of cured film from example 4 is shown in FIG. 6.

In yet another embodiment referred to as Example 5, Sol I was prepared by charging a 500 mL flask with 177 g of IPA and 25 g of 0.04 M HCl. After stirring @ 100 rpm at RT for short time (˜1 min), 2.39 g (0.01753 moles) of methyltrimethoxysilane (MTMOS) was added to the mixture. The mixture was stirred at RT for 30 min. Sol II was prepared by charging a 500 mL flask with 177 g of IPA and 25 g of 0.04 M HCl. After stirring @ 100 rpm at RT for short time (˜1 min), 0.399 g (0.00262 moles) of (3,3,3-trifluoropropyl)-trimethoxysilane (F3TMOS) was added to the mixture. The mixture was stirred at RT for 30 min. Sol I and II were mixed together followed by addition of 6.24 g (0.03 moles) of tetraethoxysilane (TEOS). The final mixture was stirred at RT for 30 min. This mixture was allowed to age under ambient conditions for 48 hours. The GPC test of the final sol produced molecular weight of Mw=1,190 g/mol with polydispersity PD=1.25. A 30×30 cm glass sheets (polished with cerium oxide polish, washed, and allowed to dry) were flow coated with the final sol mixture and allowed to dry for approximately 1-10 minutes followed by curing at temperature of 120° C. for 60 minutes. GPC results are shown in FIGS. 7d-1 and 7d-2.

In yet another embodiment referred to as Example 6, sol I was prepared by charging a 500 mL flask with 177 g of IPA and 25 g of 0.04 M AcOH (acetic acid). After stirring @ 100 rpm at RT for short time (˜1 min), 2.32 g (0.017 moles) of methyltrimethoxysilane (MTMOS) was added to the mixture. The mixture was stirred at RT for 30 min. Sol II was prepared by charging a 500 mL flask with 177 g of IPA and 25 g of 0.08 M AcOH. After stirring @ 100 rpm at RT for short time (˜1 min), 3.71 g (0.017 moles) of (3,3,3-trifluoropropyl)-trimethoxysilane (F3TMOS) was added to the mixture. The mixture was stirred at RT for 30 min. Sol I and II were mixed together followed by addition of 7.08 g (0.034 moles) of tetraethoxysilane (TEOS). The final mixture was stirred at RT for 30 min. This mixture was allowed to age under ambient conditions for 48 hours. The GPC test of the final sol produced molecular weight of Mw=2,370 g/mol with polydispersity PD=1.85. A 30×30 cm glass sheets (polished with cerium oxide polish, washed, and allowed to dry) were flow coated with the final sol mixture and allowed to dry for approximately 1-10 minutes followed by curing at temperature of 120° C. for 60 minutes. GPC results are shown in FIGS. 7e-1 and 7e-2.

In yet another embodiment referred to as Example 7, sol was prepared by charging a 500 mL flask with 354 g of IPA and 50 g of 0.08 M AcOH. After stirring @ 100 rpm at RT for short time (˜1 min), 9.13 g (0.067 moles) of methyltrimethoxysilane (MTMOS) was added to the mixture. The mixture was stirred at RT for 30 min followed by addition of 13.96 g (0.067 moles) of tetraethoxysilane (TEOS). The final mixture was stirred at RT for 30 min. This mixture was allowed to age under ambient conditions for 48 hours. The GPC test of the final sol produced molecular weight of Mw=926 g/mol with polydispersity PD=1.32. A 30×30 cm glass sheets (polished with cerium oxide polish, washed, and allowed to dry) were flow coated with the final sol mixture and allowed to dry for approximately 1-10 minutes followed by curing at temperature of 120° C. for 60 minutes. GPC results are shown in FIGS. 7f-1 and 7f-2.

In yet another embodiment referred to as Example 8, sol was prepared by charging a 500 mL flask with 354 g of IPA and 50 g of 0.08 M HCl. After stirring @ 100 rpm at RT for short time (˜1 min), 9.13 g (0.067 moles) of methyltrimethoxysilane (MTMOS) was added to the mixture. The mixture was stirred at RT for 30 min followed by addition of 13.96 g (0.067 moles) of tetraethoxysilane (TEOS). The final mixture was stirred at RT for 30 min. This mixture was allowed to age under ambient conditions for 48 hours. The GPC test of the final sol produced molecular weight of Mw=805 g/mol with polydispersity PD=1.12. A 30×30 cm glass sheets (polished with cerium oxide polish, washed, and allowed to dry) were flow coated with the final sol mixture and allowed to dry for approximately 1-10 minutes followed by curing at temperature of 120° C. for 60 minutes. GPC results are shown in FIGS. 7g-1 and 7g-2.

In yet another embodiment referred to as Example 9, sol was prepared by charging a 500 mL flask with 345 g of IPA and 50 g of 0.08 M HCl. After stirring @ 100 rpm at RT for short time (˜1 min), 9.13 g (0.067 moles) of methyltrimethoxysilane (MTMOS) was added to the mixture. The mixture was stirred at RT for 30 min followed by addition of 13.96 g (0.067 moles) of tetraethoxysilane (TEOS). The final mixture was stirred at RT for 30 min. This mixture was allowed to age under ambient conditions for 48 hours. 10.5 mL of 1-methoxy-2-propanol was added to the sol mixture and stirred for 1 min. The GPC test of the final sol produced molecular weight of Mw=857 g/mol with polydispersity PD=1.12. A 30×30 cm glass sheets (polished with cerium oxide polish, washed, and allowed to dry) were flow coated with the final sol mixture and allowed to dry for approximately 1-10 minutes followed by curing at temperature of 120° C. for 60 minutes. GPC results are shown in FIGS. 7h-1 and 7h-2.

In yet another embodiment referred to as Example 10, sol was prepared by charging a 500 mL flask with 197 g of IPA and 140 g of 0.08 M HCl. After stirring @ 100 rpm at RT for short time (˜1 min), 30.2 g (0.288) of methyltrimethoxysilane (MTMOS) was added to the mixture. The mixture was stirred at RT for 30 min followed by addition of 60 g (0.288 moles) of tetraethoxysilane (TEOS). The final mixture was stirred at RT for 30 min. This mixture was allowed to age under ambient conditions for 48 hours. The GPC test of the final sol produced molecular weight of Mw=1,635 g/mol with polydispersity PD=1.40. A 30×30 cm glass sheets (polished with cerium oxide polish, washed, and allowed to dry) were flow coated with the final sol mixture and allowed to dry for approximately 1-10 minutes followed by curing at temperature of 120° C. for 60 minutes. GPC results are shown in FIGS. 7k-1 and 7k-2.

Where applicable, the measurement of anti-reflective properties of the coatings was done as follows: The transmittance of the coatings was measured by means of UV-vis absorption spectrophotometer equipped with an integrator accessory. The anti-reflective enhancement factor is measured as the relative percent increase in transmittance compared to untreated glass slides versus glass slides coated with compositions of this disclosure. ASTM E424 describes the solar transmission gain, which is defined as the relative percent difference in transmission of solar radiation before and after the application of the coating. The coatings exhibit about 1.5% to about 3.25% gain in solar transmission. The refractive index of the coating was measured by an ellipsometer.

The abrasion resistance of the coating is measured by an abrader device according to European standard EN1096.2 (glass in building coated glass). The coatings made according to Examples 1, 2, 3, 4, 5, 6, 7, 8, 9, and 10 without any added composition modifying additives, are able meet the passing criteria of the standard. Coatings made from Example 3, 7, 8, 9, 10 are exceptional in that it is able to have almost no damage after 500 cycles of testing per the EN1096 standard. Abrasion losses are less than 0.5%.

The contact angle of the coatings is measured by means of goniometer wherein the contact angle of the water droplet is measured by means of a CCD camera. An average of three measurements is used for each sample. On tin-sided float glass, average contact angles for coatings made from Examples 2, 4, 5 and 6 measure 85° and on tin-sided TCO glass, average contact angles measure 90°.

The reliability results of the coatings in this disclosure are broadly similar to existing anti-reflective coatings. However, under 85° C./85% RH test conditions per IEC61215 and IEC61646 the coatings of this disclosure have a protective effect on glass corrosion which is not observed when highly porous anti-reflective sol-gel coatings are tested under similar conditions. Without being bound to theory, we believe that porous anti-reflective coatings facilitate easy leaching of sodium ions from the glass whereas the coatings of this disclosure can be tuned to achieve hydrophobic properties which slow down the rate and/or decrease the amount of water that is contact with the glass. Coatings made from examples 2, 4, 5 and 6 of this disclosure exhibit minimal glass corrosion compared to uncoated glass. The other remarkable feature of the passing reliability results is that these reliability results have been achieved with a coating cured at just 120° C. Existing anti-reflective coatings are typically sintered at 400˜700° C. to achieve the level of reliability indicated by these results.

FIG. 1a illustrates the UV-vis transmittance spectra showing maximum transmittance enhancement of coatings on tin side of float glass from composition given in Example 2. A statistical comparison of 11 samples from coating made from composition in Example 2 on tin side vs non-tin side of float glass provided a solar weighted photon gain of 2.23% vs 1.93%. Without being bound to theory, the coatings of this disclosure interact with the tin side of float glass to provide an enhancement in the beneficial properties of the antireflective coatings.

FIG. 1b illustrates the UV-vis transmittance spectra of roll coated coating made from Example 3 on patterned glass substrate

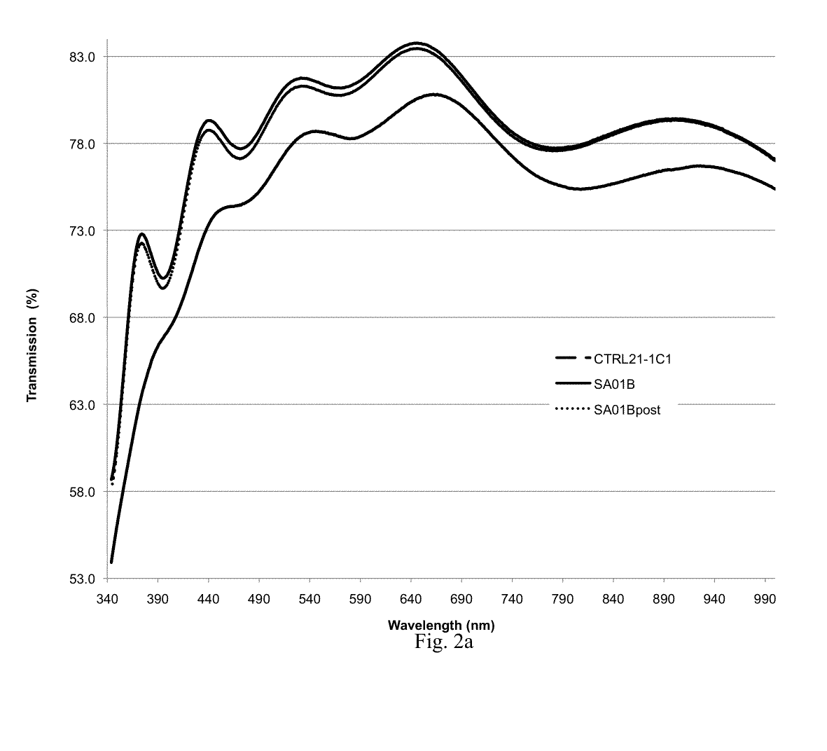

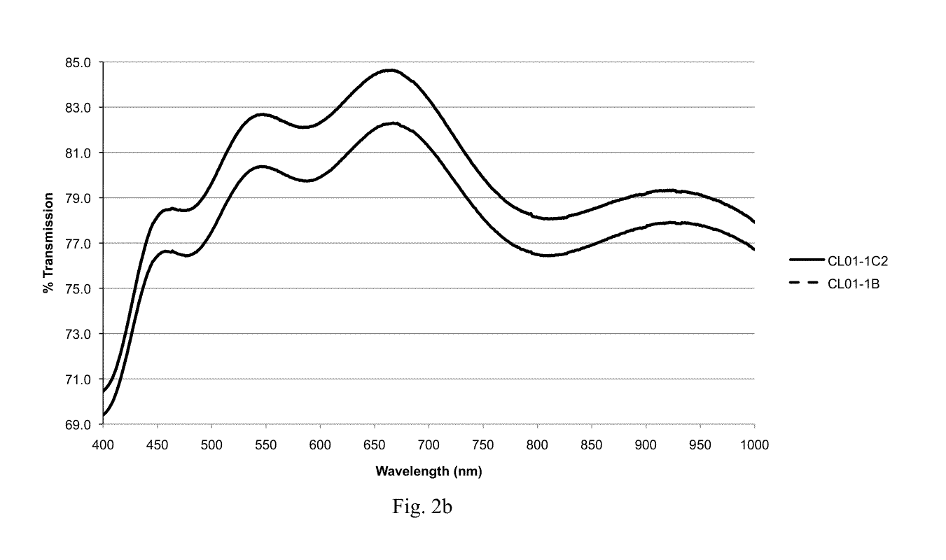

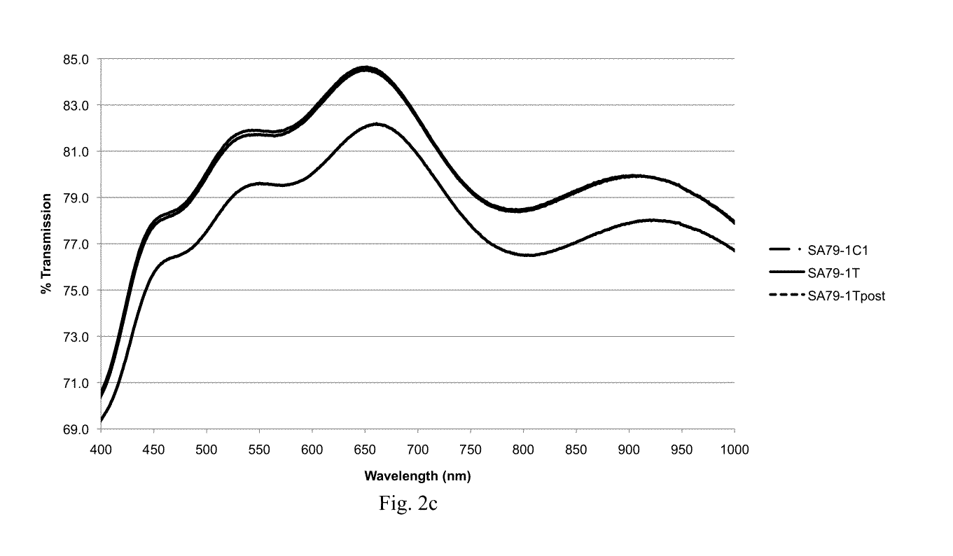

FIG. 2a illustrates the UV-vis transmittance spectra showing maximum transmittance enhancement of coatings on tin side of TCO glass substrates made from compositions given in Example 3 comparing pre- and post-abrasion spectra.

FIGS. 2b and 2c illustrates the UV-vis transmittance spectra of coatings made from example 5 and Sols from the three formulations could have different inherent viscosities and it would be preferable to be able to tune the viscosities of the sols such that their solar weighted photon gain is maximized.

FIG. 3a is a TEM cross-sectional view of a coating made from the composition of Example 2 on a glass slide substrate. The TEM images show the absence of any discernible porosity in these coatings. The film thickness about 70-80 nm.

FIG. 3b is a HRTEM of a coating made from the composition of Example 2 on a glass slide.

FIG. 4 is an SEM cross-sectional view of a coating made from the composition of Example 2 on a 30×30 cm float glass substrate. The SEM images show the absence of porosity and a film thickness of 133 nm.

FIG. 5 is an SEM cross-sectional view of a coating made from the composition of Example 3 on a 30×30 cm float glass substrate. The SEM images show the absence of porosity and a film thickness of ˜83 nm.

FIG. 6 is an SEM cross-sectional view of a coating made from the composition of Example 4 on a 30×30 cm float glass substrate. The SEM images show the absence of porosity and a film thickness of 76 nm.

It should be appreciated that in some embodiments, the coatings of the present disclosure provide an increase in transmission from about 1% to about 3.5% and in some embodiments from about 1.5% to about 3%, and a contact angle of about 80 degrees to about 120 degrees and in some embodiments about 85 degrees to about 100 degrees.

Typical hardness for a mixture of pure silica sol-gels coatings is observed to be around 1.05 GPa. Without wishing to be bound by theory, the enhanced mechanical properties of the coatings of this disclosure (as compared to pure silica-based coatings) may be due to several factors that contribute to increased hardness. First, the extensive cross-linking due to the use of the three-precursor system makes the Si—O—Si network stronger. Second, the combined use of organosiloxane and organofluorosiloxane enhances the noncovalent interactions between the organic side chains to promote better interactions that enhance the overall mechanical properties. Third, the increased interactions between the side chains promote a better filling of porous void space in the sol-gel network to make a homogenous and largely nonporous coating. Taken together, the unique combination of precursors along with the absence of visible porous microstructure and the enhanced side chain interactions between the organic groups provides the improved mechanical properties as compared to coatings of the prior art.

The anti-soiling and self-cleaning property of coatings of this disclosure can be tuned by changing the surface characteristics of these coatings. XPS data for example coatings of this disclosure show how the fluorine content of the coatings can be varied from 0-9.1% and carbon content can be varied from 16.8% to 41.7%.

TABLE 5
Showing XPS Data for Coatings of this Disclosure on Tin Side of TCO Coated Glass
Subject to 20 sec Ar+ sputter to remove any adventitious impurities

| Sample | F % | C % | Si % | O % | N % | Na % | Ca % |
|---|---|---|---|---|---|---|---|
| Example 2 + 20 sec sputter | 9.1 | 41.7 | 16.1 | 32.1 | nd | 0.6 | 0.4 |
| Example 3 + 20 sec sputter | nd | 16.8 | 28.5 | 53.6 | nd | 0.7 | 0.3 |
| Example 4 + 20 sec sputter | 7.4 | 25.3 | 21.7 | 44.1 | 0.5 | 1.0 | nd |

TABLE 6
Showing XPS Data for Coatings of this Disclosure in the native state and after 10 minutes
of Argon Sputter Etch on Tin Side of TCO coated Glass

| Sample | F % | C % | Si % | O % | Sn % | Na % | Ca % |
|---|---|---|---|---|---|---|---|
| Example 2 as received | 11.9 | 39.5 | 14.9 | 32.7 | nd | 0.6 | 0.4 |
| Example 2 after 10 min sputter | 12.9 | 13.1 | 28.7 | 45.1 | 0.2 | Nd | nd |

A comparison of the XPS data for the as received sample from Example 2 and the XPS data for the same sample after it was sputtered with Argon ions for 10 minutes show that Fluorine from the coating material is present in the as received sample and after the 10 minute etch. The data also shows that small amount of tin from the tin side of the TCO coated float glass are detected along with the coating.

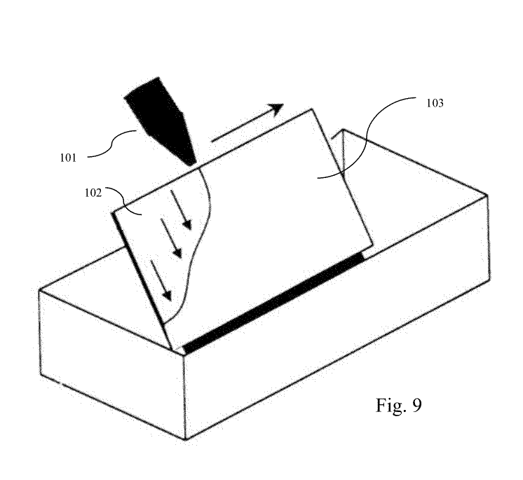

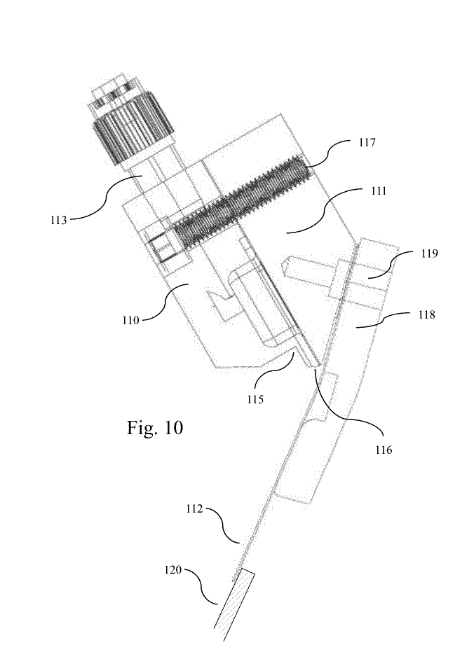

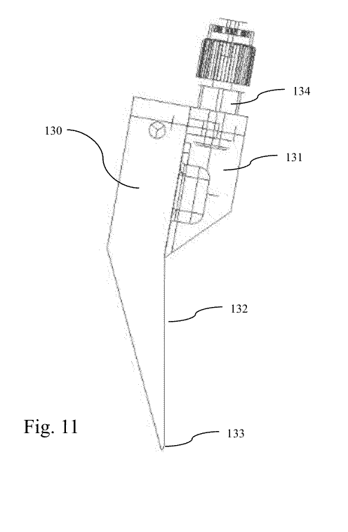

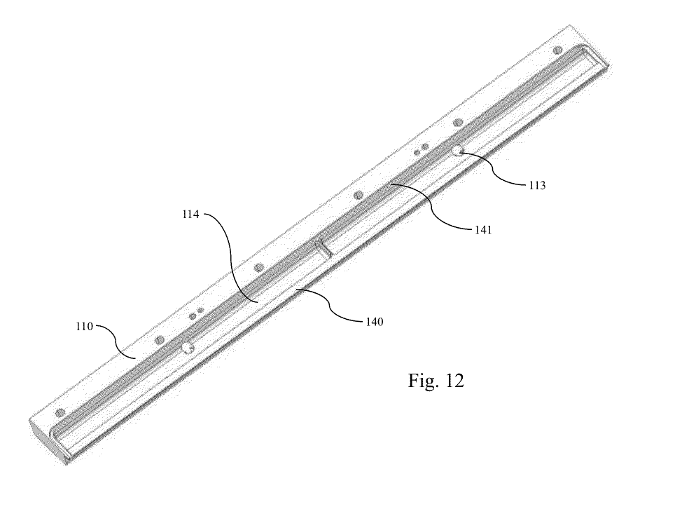

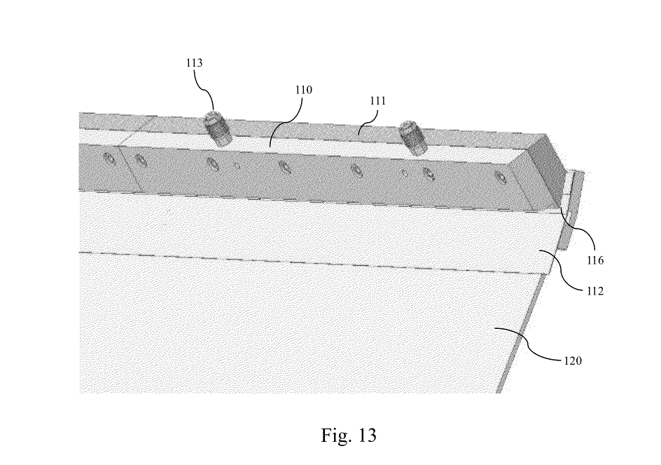

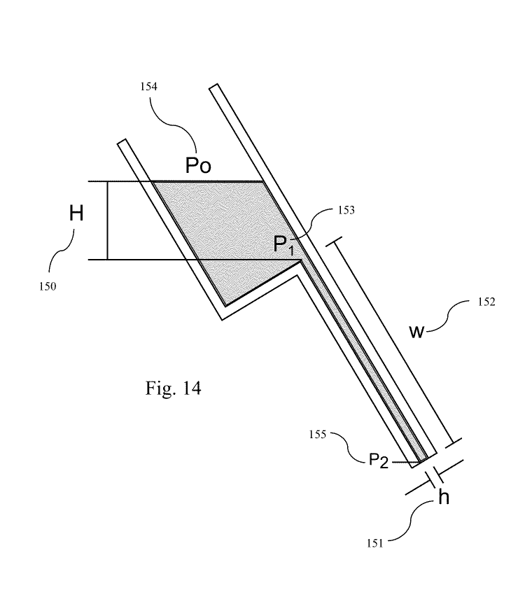

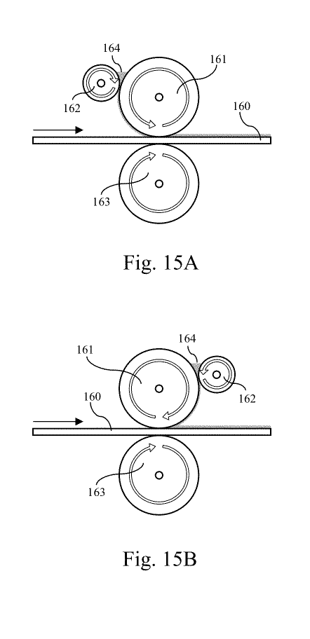

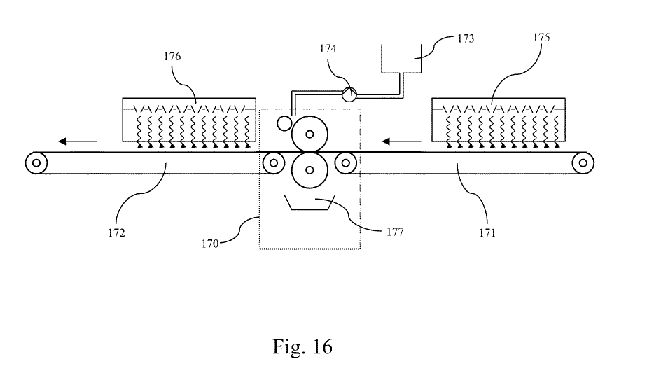

As indicated elsewhere herein, the coatings of this disclosure may be deposited on substrates by techniques known in the art, including dip coating, spraying, drop rolling, or flow coating to form a uniform coating on the substrate. A flow coating head, as depicted in FIG. 9, FIG. 10, FIG. 11, FIG. 12, FIG. 13, and FIG. 14 may enable improvements in flow coating. A process for roll coating is described in FIG. 15A, FIG. 15B, and FIG. 16.

FIG. 9 depicts an embodiment of laboratory scale flow coating. In embodiments, a nozzle (101) dispenses a material (102) onto an inclined substrate (103) as it is moved across the top edge of the substrate. The material flows down the substrate, and the excess drips from the bottom edge of the substrate. The material that remains adhered to the substrate undergoes a gelation process as it dries and forms a thin-film coating on the substrate.

While the basic laboratory system shown in FIG. 9 can be scaled up in substrate size, its rate of coating may be slow and wasteful of coating material. It is possible to recover the coating material that drips off the bottom edge and recycle it to the nozzle, but this makes control of composition and contamination of the recycled material difficult. What is needed is a flow coating system that has a fast coating rate and that is economical with coating material with minimal wastage dripping from the bottom edge, without recycling of this material.

In one embodiment, a coating head such as the one shown in FIG. 13 and in cross-section in FIG. 10 may be used in flow coating. The coating head includes a long slot (116) formed between a lower slot manifold (110) and an upper slot manifold (111). This slot is positioned parallel to and extends along the length of the top edge of an inclined substrate (120). In an embodiment, the slot is approximately as long as the edge of the substrate to be coated. For example, the slot may be oriented along the longer edge of a rectangular substrate, such that the fluid flows down the substrate along its shorter edge. This orientation minimizes the time required for gravity to carry the fluid across the entire area of the substrate. In an embodiment, a distribution blade (112) bridges the gap between the slot and the top edge of the substrate such that coating material flowing out of the slot is deposited on to the distribution blade and then flows under gravity to the bottom of the distribution blade, which contacts the front surface of the substrate just below the top edge of the substrate. The coating material then flows off the distribution blade onto the front surface of the substrate and from there down the substrate until eventually it either drips from the bottom edge or is removed by other means. The length of the distribution blade is slightly longer than the length of the slot and of the edge of the substrate that is being coated. In an embodiment, the distribution blade extends beyond each end of the slot manifold assemblies. For example, the distribution blade may extend 2-100 mm beyond each end of the slot manifold assemblies. In another example, the distribution blade may extend 10 mm beyond the substrate.

Coating material is supplied to the slot by a dispensing system, such as a pump (not shown) capable of transferring the liquid coating material, and that is also capable of delivering a measured quantity of coating material through one or more inlet ports (113) in the lower slot manifold. The inlet port directs material into a corresponding internal pocket (114) within the lower slot manifold that allows the coating material to accumulate below the lip of the slot and to spread evenly along the slot before it begins to overflow the slot and flow onto the distribution blade, providing a uniform fluid front of material over the blade. FIG. 12 shows an isometric view of the internal detail of a lower slot manifold (110). The coating material flows from the port inlet, located in the middle of the internal pocket, outwards toward the ends of the internal pocket and so is distributed evenly along the back side of the slot lip (140). Once enough material has filled the internal pocket it will begin to overflow the slot lip evenly along the length of the slot. The upper slot manifold (not shown in FIG. 12) forms the opposing side of the slot. A seal channel (141) may allow the assembly to close to the appropriate slot width, as is described herein.

Producing high quality coatings of uniform thickness onto the substrate may depend on the rate at which the fluid flows through the slot. In turn, the rate at which the material flows may be highly dependent upon several factors of the design including the slot length (l), width (w) (152) and height (h) (151), as seen in FIG. 14, the viscosity (p) and density (p) of the coating material, and the pressure differential (ΔP) over the width of the slot. In an embodiment, the fluid flow in the slot is both laminar and has a fully developed velocity profile upon exit onto the distribution blade. Laminar flow in the slot can be achieved by ensuring the fluid has a Reynolds number less than 1,400. In an embodiment, the Reynolds number (Re) of the coating fluid within the slot is less than 100. The coating fluid may exit the slot with a velocity profile that is independent of subtle edge effects, turbulence and other disturbances present at the coating fluid's entry into the slot. This condition can be achieved by ensuring the width of the slot is significantly longer than the flow's characteristic entrance length (Le). In an embodiment, the slot width is equal to at least 10 times the entrance length. Such a condition is governed in the following relation, which uses the Blasius approximation to solve for the entrance length between parallel surfaces:

$${L}_{e} = \frac{{hRe}_{h}}{100}$$

The volumetric rate at which the coating fluid flows through the slot is closely approximated by the following relation:

$$Q = \frac{l Δ {Ph}^{3}}{12 w μ}$$

With average flow speed, V, determined by:

$$V = \frac{Q}{lh}$$

In an embodiment, sol coating flow rates per unit slot length of between 5×10⁻⁹ and 5×10⁻⁴ m²/s are useful for coating glass substrates of high quality, and uniform thickness. In an embodiment with a 2 meter long slot, this equates to a volumetric flow rate between 1×10⁻⁷ and 1×10⁻³ m³/s. To prevent splatter or turbulent flow or other undesirable phenomena from impacting the distribution blade or substrate, coating material may not be forced from the slot under high pressure or flow rates. For example, gravity force may be used to drive fluid from the internal pocket to the distribution blade. In an embodiment, the slot is designed such that for the chosen coating material properties, the flow rate out of the slot is less than the flow rate into the internal pocket. This has the effect of building a reservoir of coating material behind the slot in the internal pocket, forcing it to spread evenly under the influence of gravity along the entire length of the slot and to build up a head height H (150), as in FIG. 14, inside the internal pocket. If the flow rate through the slot is too high, then coating material will completely flow through part of the slot before spreading along the entire length of the slot and reaching the ends furthest away from the inlet port. If the flow rate is too low, then the internal pocket may completely fill with coating material causing an increase in pressure that will create uneven flow rates and excessive back pressure on the coating fluid, and adversely affect the flow rate through the slot. All of these issues can cause the slot flow rate to vary and can affect the quality and uniformity of the coating. The pressure drop over the slot width, ΔP, can be related the fluid head height within the interior pocket, H (150), the internal pocket pressure Po (154), pressure at the entrance to the narrow slot, P1 (153), and the pressure at the exit of the slot, P2 (155), the fluid material density ρ and the gravitational constant g according to the following relationship:

ΔP=P₁−P₂

ΔP=ρgH+Pₒ

This pressure input as a function of head height, combined with the desired flow rate drives the desired slot height, h (151). As a result, careful consideration should be paid to the pressure in the internal pocket. Some embodiments keep the internal pocket sealed via a gasket, o-ring or sealant such that pressure is controlled by the relative flow rates of coating material into and out of the pocket. Other embodiments may include vents between the internal pocket and ambient pressure or to an auxiliary pressurization system. In an embodiment, pressure inside the pocket is vented to the atmosphere and slot height, h, is determined by the following relationship:

$$h = \sqrt[3]{\frac{12 Qw μ}{l ρ gH}}$$

Given the above parameters, for a typical sol coating, the width of the slot is between 0.05 and 2 mm, and preferably 0.1 to 0.5 mm. This width may be controlled by placing shims between the upper and lower slot manifolds. Alternative embodiments may use machined steps or other gap control methods. The assembly of upper and lower slot manifolds may have a gasket-like seal along the top and sides to ensure material is directed towards the slot. An O-ring or similar internal pocket seal may allow the assembly to close to the appropriate slot width, and may be facilitated with the use of a seal channel (141).

The distribution blade may serve at least three functions in enabling consistent and uniform coating thickness; 1) it provides a path for coating material to flow from the slot to the substrate; 2) it has a high energy surface that causes the material to spread evenly by surface tension during its travel from the slot to the substrate; and 3) it provides an interface to the substrate surface that is tolerant of imperfections in flatness or warping of the substrate. In one embodiment, the distribution blade is relatively more flexible than the substrate and is able to conform to an uneven or warped substrate. For example, the distribution blade is 316L stainless steel, 2020 mm long, 45 mm wide and 0.38 mm thick and the substrate is tempered soda-lime glass 1970 mm long, 984 mm wide and 3.2 mm thick. In another embodiment, the distribution blade is relatively more rigid than the substrate and a mechanism clamps the substrate to the back surface such that it is held flat against the distribution blade. In one embodiment, the distribution blade has a surface energy between 25 mN/m and 100 mN/m.

The coating material exiting the head slot may not naturally form a continuous curtain or ‘waterfall’ of coating material in the absence of the distribution blade, and instead, the coating material may exit the slot with many drips or small rivulets of material all along the length of the slot which may not result in a consistent or uniform thickness coating on the substrate. To achieve a curtain or “waterfall” out of the slot head in the absence of the distribution blade would require significantly greater flow rates of coating material, and could therefore result in significant waste of coating material. Thus, the distribution blade enables a consistent and uniform thickness coating with minimal material waste.

In FIG. 10, the distribution blade is a thin piece of material that is held in place by a backing plate (118) that along with the distribution blade is attached to the upper slot manifold (111) by a plurality of bolts or other fastening means (119). This backing plate also serves to tension the distribution blade by forcing it forward at a slight angle. This reduces warping of the thin distribution blade along its length. The upper and lower slot manifolds are held together by a plurality of bolts or other fastening means (117). In some embodiments the bottom edge of the thin distribution blade may be beveled or rounded. In a preferred embodiment it is beveled between 15° and 60°.

In some embodiments the distribution blade is made from a stainless steel alloy such as 316L. In other embodiments it could be made from titanium, chrome or nickel plated steel, various corrosion resistant alloys, glass, ceramics, polymer or composite materials such as a metal coated polymer. The material may be chosen to be chemically resistant to the composition of the coating material such that it is not damaged by the coating material and such that it does not contaminate the coating material in any way.

In FIG. 10, the lower slot manifold has a notch (115) just below the slot. The purpose of this notch is to prevent the flow of coating material from the slot along the bottom edge of the lower slot manifold and from there dripping on to the distribution blade or the substrate.

FIG. 11 shows an alternative embodiment of a distribution blade (130) wherein the blade is a solid piece of material that also forms the upper slot manifold. The front surface of the blade (132) acts to distribute the coating material evenly from the slot to the substrate. The bottom edge of the blade is profiled (133) to facilitate the flow of coating material from the blade onto the substrate. It should be understood that the exact shape of this profile can include curved or angled flat bevels and that the transition of angle from the face of the distribution blade can range from gradual to abrupt and that the final angle that the edge makes with the substrate surface can be from 10° (sharp) to 110° degrees (obtuse). In another embodiment, the thick or solid distribution blade does not also form the upper slot manifold, but is instead a separate piece that is bolted onto the slot manifold in a manner similar to the thin distribution blade shown in FIG. 10.

Some embodiments of the distribution blade include coatings or surface treatments on the front side (that is the wet side) and on the back side. For example, a front side surface treatment may enhance the spreading of the coating material as it flows to the substrate. A back-side treatment might repel the coating material to suppress material gathering on the backside due to capillary action that then dripped onto the substrate as it was removed from the distribution or gather on the backside and contaminate the next substrate positioned against the blade. Other embodiments of the distribution blade include laminates and composites where dissimilar materials are fused or assembled together to provide differences between the front and backside surface properties as might also be achieved in the case of a coated metal blade.

Some embodiments of the coating head manifolds may have coatings or surface treatments to protect them from adverse chemical reactions with the coating material or to change how the coating material flows within the internal pocket or over the slot lip.

A full coating head may be composed of a plurality of slot manifold assemblies. For example each slot manifold assembly might be 50 cm long. Four such assemblies may be mounted on a supporting structure such that they form a 200 cm long coating head. The dimensions of the slot manifold assembly and the number of such assemblies used for a particular length of coating head may be selected to manage the cost of manufacturing the slot manifolds themselves and the complexity of constructing the coating head from multiple slot manifold assemblies. In the case where multiple slot manifold assemblies are used to assemble a coating head, it is advantageous to have a single distribution blade that is continuous over the entire length of the coating head. However, multiple adjacent or overlapping segments of distribution blade comprising the length of the coating head are not precluded.

It should be understood that the number of internal pockets and inlet ports within a slot manifold is variable and may be more or less than the two shown in FIG. 12. The number of pockets and inlet ports may be selected to manage the manufacturing complexity of the slot manifold and the uniformity of flow of coating material from the slot.

In the slot manifold, the wall between internal pockets may be kept as thin as possible. This wall affects the flow of material over the slot lip in its immediate vicinity. By keeping the wall as thin as is practical, the effect is minimized.

The method of coating using the apparatus may include the following steps. First, optionally, the substrate may be prepared for the coating by increasing the surface energy of the surface to be coated, thus making it possible for the coating material to spread evenly on the substrate surface by surface tension. In one embodiment, the substrate is glass and the surface energy is increased by washing vigorously with water and/or mechanical brushes. In other embodiments, the substrate surface may be prepared using gas plasma such as oxygen or by treatment with a gas flame. Other pretreatments are described further herein.

Next, the substrate to be coated may be positioned with its top edge aligned with and parallel to the bottom edge of the distribution blade. The bottom edge of the distribution blade may overlap slightly with the top edge of the substrate. The amount of overlap is dependent upon the coating requirements but may be at least 0.1 mm and in a preferred embodiment be approximately 3 mm. The ends of the distribution blade may extend slightly beyond the left and right edges of the substrate, between 2 and 100 mm on each side. In an embodiment, it extends by 10 mm on each side. The substrate may be inclined at an angle of 60° to 85° relative to horizontal. In the case of a flexible thin distribution blade, the angle between the surface of the substrate and the surface of the distribution blade may be between 0° and 5°. The substrate can be pushed slightly against the distribution blade to apply pressure to the contact area such that the distribution blade conforms to any gross irregularity or deviation from flatness of the substrate. In the case of a rigid distribution blade, the substrate may be positioned with its front surface parallel to the back surface of the distribution blade and a clamping mechanism may hold the substrate to the distribution blade such that any warping or deviation from flatness of the substrate is eliminated against the flat back side of the distribution blade. In one embodiment, the coating head is stationary and the substrate is brought to it. However, in other embodiments, the substrate may be stationary and the coating head moved to position or both elements may move together to arrive at the final coating position. It is also possible for both elements to be stationary relative to each other but to be moving relative to the larger coating system.

Next, the front surface of the substrate may be completely wetted with a pre-wet solution. This pre-wet solution is dispensed in a manner that quickly wets the entire substrate surface rapidly, such as in less than 30 seconds. In one embodiment, a plurality of fan nozzles positioned on a rotatable mechanism above and in front of the substrate and along its length aligned to the coating head starts spraying pre-wet solution such that it first wets the distribution blade along it entire length. Then the nozzle assembly rotates such that the fan shaped jets of pre-wet solution from the nozzles travel down the substrate from its top edge to its bottom edge and in the process deposit pre-wet solution on the full surface of the distribution blade and the substrate. When employed, the pre-wet step decreases the time for the coating material to completely wet the substrate to between 1 and 25 seconds; improves the uniformity of distribution of the coating material on the substrate to ±25% by volume per unit area and reduces the amount of coating material needed to completely coat the substrate by up to 90%. The composition of the pre-wet solution is chosen to provide a number of properties: The viscosity is within ±50% of the viscosity of the coating material and more preferably within ±10% and even more preferably within ±2% and/or the surface tension is within ±50% of the surface tension of the coating material and more preferably within ±10% and even more preferably within ±2% and/or the vapor pressure is within ±50% of the vapor pressure of the coating material and more preferably within ±10% and even more preferably within ±2%. In one embodiment, the pre-wet solution comprises the same mixture of solvents, mixed in the same ratios as the coating material. For example, the pre-wet solution might be composed of 90% isopropyl alcohol and 10% water that approximately matches the ratio of isopropyl alcohol and water in a sol-gel coating material. In an alternative embodiment, the pre-wet solution could be a non-ionic, cationic or anionic surfactant, such as for example sodium dodecyl sulfate or perfluoroalkyl sulfonate.

Next or some time shortly after the pre-wet step has commenced, a pre-determined amount of coating material may be dispensed from the coating head on to the substrate. The coating material flows down the substrate completely covering the front surface of the substrate. Excess coating material may drip from the bottom edge or be wicked away from bottom edge by capillary action onto a mechanism designed for that purpose. In some embodiments, excess coating material may be collected at the bottom of the substrate for reuse. The decision to reuse this material or not depends on the composition of the coating material and substrate. For example, if the coating material is quite stable and does not significantly change during the time it travels down the substrate and if the substrate does not contaminate the coating material then a decision might be made to reuse excess material collected from the bottom edge.

Next, optionally, there may be a pause of between 1 and 600 seconds after the dispensing of coating material has finished while excess coating material is able to drain out of the internal pocket and from the wet surface of the distribution blade onto the substrate. The length of this pause may be optimized to reduce the possibility of drips from the distribution blade after the substrate is removed from the coating head. In some embodiments, this pause may be long enough to allow the distribution blade and/or the top area of the substrate to dry or partially dry.

Next, the substrate may be withdrawn from the coating head. In some embodiments, if the coating head is still wet, a drip guard may quickly move into place between the substrate and the bottom edge of the distribution blade. This drip guard may optionally touch the bottom edge of the blade to wick away excess material in which case the surface of the drip guard may have similar surface characteristics to the front surface of the distribution blade to encourage the coating material to easily flow off the distribution blade.

Finally, the substrate may be allowed to dry in a manner that allows the coating material to undergo gelation such that a uniform high quality coating is formed on the substrate surface.

This coating method, enabled by the novel design of the coating head can have several of the following advantages over alternative coating techniques. First, by dispensing material simultaneously across the full width of the substrate the time to dispense can be greatly shortened. Second, by pre-wetting the substrate the amount of time for the coating material to flow down the substrate can be greatly shortened and the amount of coating material required to fully wet the substrate surface is greatly reduced. Third, if coating material is not collected at the bottom of the substrate for reuse then only fresh (virgin) material can be deposited on the substrate so control of coating material purity and composition can be greatly increased. Fourth, by utilizing a distribution blade in conjunction with a properly sized slot dispenser, the uniformity of flow of material on to the substrate can be greatly increased at very low cost and with a very simply system. Fifth, the technique can be very tolerant of deviation of flatness on the substrate without requiring any precision mechanical control or design. Sixth, the method does not necessarily pose any significant chemical compatibility challenges where it may be difficult to identify critical coating components with properties that are not sensitive to or contaminate the coating material. Finally, the method can be inherently single sided allowing the flexibility to coat one side of the substrate or both (in a second coating step) if needed.

Is should also be understood that in some embodiments the formulation of the coating material will have a significant effect on the uniformity of the thin-film. In particular, in a sol-gel coating material the ratio of solids or particle content to solvent in conjunction with the ambient conditions during drying may affect the gelation process that occurs as the thin-film forms. Careful control of these elements will enhance the uniformity of the final thin-film especially in the top to bottom direction on the substrate.

FIG. 15a shows a simplified schematic of a forward roll coating apparatus. FIG. 15b shows a simplified schematic of a reverse roll-coating apparatus. In both figures, a flat substrate (160) is fed from left to right. A counter pressure roller (163) supports the substrate from the bottom and moves in a complementary direction to the movement of the substrate. A coating material (164) is deposited in a reservoir created between a doctor roller (162) and an application roller (161). The pressure or spacing of the doctor roller to application roller controls the amount of coating material that is transferred to the application roller. The surfaces of the doctor and application rollers may be smooth or textured, soft or hard. The roller surfaces need not be the same. For example, the doctor roller may be compliant and textured while the application roller could be hard and smooth and vice versa. The application roller transfers coating material to the surface of the substrate. The pressure or distance between the application roller and the substrate surface is adjustable to facilitate control of the final wet-coating thickness and/or uniformity of the material on the substrate. In forward roll-coating, the application roller (161) moves in the same direction as the direction of motion of the substrate. In reverse roll-coating, the application roller (161) moves in the opposite direction to the motion of the substrate.

The substrate may be continuous, such as for example a roll of polymer sheet or steel, or it may be discontinuous, such as discrete pieces of glass or wood or individual solar modules. In the case of discontinuous substrates, the application roller assembly may be moved in a vertical direction such that it touches down on the leading edge of the substrate as it enters the roll-coater and then lifts off the trailing edge as the substrate exits the roll-coater. This technique may reduce uniformity on the leading and trailing edges.

The selection of the materials within the roll-coater that come into contact with the liquid coating material are a consideration. In some embodiments, the coating material may be corrosive, having either a high or low pH. In an embodiment, the pH of the coating material is between 1.8 and 2.8. Additionally, in some embodiments, the coating material contains organic solvents such as isopropyl alcohol, methanol, ethanol, propylene glycol monomethyl ether, propylene glycol monomethyl ether acetate, and the like. All materials may be selected to withstand both the organic solvents and pH conditions used. For metallic components, stainless steel is preferential with chrome-plated steel, for example. In selecting polymer materials for pipes, fittings and seals made from polytetrafluoroethylene, polypropylene, polyether ether ketone, and polyvinylidene difluoride may be considered. For polymer coatings on the rollers polyurethane, EPDM (ethylene propylene diene monomer) rubber and nitrile rubber are suitable. The particular embodiment of a roll-coater selected for a specific sol-gel coating application depends upon a number of factors. The wet film thickness is a process parameter to consider in achieving the final cured film thickness. The desired wet thickness may be dependent on the desired final dry thickness, the solids content of the coating material and the target porosity of the final dry film. In one embodiment, the desired final thickness is 120 nm (DT), the solids content (SC) of the coating material is between 1% and 3% by volume and the target porosity (P) is 10%. The target wet thickness (WT) may be calculated with the following formula:

$$WT = \frac{DT}{SC * ( 1 - P )}$$

For example, the equation yields a target wet thickness between approximately 4 μm and 14 μm using the input parameters above. Wet thickness can be controlled by a number of process controls on the roll-coater system. Selection of which parameters are most important is dependent upon the characteristics of the coating material, such as for example its viscosity, and the architecture or operation mode of the roll-coater, such as forward or reverse. Typically, the parameters adjusted are the doctor roller spacing and/or pressure to the application roller; the application roller spacing/pressure to the substrate; the speed at which the substrate moves and in the case of reverse roll-coating the difference in speed between the substrate and the application roller. The speed at which the doctor roller moves relative to the application roller is also a process parameter. FIG. 16 shows an embodiment of a roll-coater used for sol-gel coating of flat substrates such as glass or solar modules. The roll-coater (170) is positioned after a feed-in conveyor (171) and ahead of a feed-out conveyor (172). In FIG. 16, substrates move from right to left. Coating material (173) is fed to the roll-coater from a storage tank at a controlled rate by a pump (174). Excess material is collected (177) off the ends of the rollers and recirculated. An optional pre-heater (175) may be positioned such that it can heat the substrate prior to the roll-coater. The substrate may be heated to a temperature, such as a temperature between 2° C. and 80° C. In some embodiments, this pre-heat step can serve to reduce thermal stress during the very rapid heating of subsequent process step. In other embodiments, it is used to control evaporation rates of the coating material placed on the substrate to achieve specific process targets such as uniformity, film-thickness, porosity or process speed. Careful consideration should be paid regarding heat transfer from warmed substrates to the application roller such that it is accounted for in the process. In one embodiment, a flash-off heater (176) is positioned at the output of the roll-coater to control evaporation of the solvent of the coating material to facilitate the gelation of the thin-film. In some embodiments, the pre-heater and the flash-off heater may be radiant infra-red or in other embodiments they may be electric or fuel fired convection heaters. In another embodiment forced air at ambient or close to ambient temperature could be used to accomplish the flash-off process by accelerating solvent evaporation.

The conveyor systems used to move substrates between process stages may be continuous belt driven systems. In some embodiments robots might be used to convey substrates between process stages. In other embodiment substrates might be conveyed by humans using carts. In any case it should be understood that substrates may be conveyed between process steps by many means known in the art.

An important consideration when using roll-coaters is accommodating or controlling for evaporation of coating material solvent from the equipment itself as the machine is running To mitigate this evaporation, it can be advantageous to add make-up solvent to the coating material such that the solids concentration is controlled within a workable range. Make-up solvent can be added at a constant rate known to match the steady-state rate of evaporation; it can be added periodically based on pre-determined intervals based on time, quantities of substrates coated, or coating material consumed. Make-up solvent can be added based on an active feedback loop wherein the solids concentration is measured directly or indirectly and then used to control the amount added. Solids concentration might be measured by optical means such as dynamic light scattering or adsorption or refractive index; it could be measured by physical properties such as for example density or viscosity; it could be measured chemically such as for example monitoring pH.

Sol-gel materials used for coatings are often sensitive to environmental conditions such as relative humidity and temperature during the gelation process. Additionally, sol-gel materials may release significant amounts of solvent vapor prior to or during cure. It is therefore desirable to engineer the environment around the roll-coating system such as that temperature and humidity are controlled, and solvent vapor is removed. In some embodiments a containment chamber is built around the complete roll-coater system with a dedicated HVAC unit to control temperature and relative humidity. In an embodiment, there is a secondary interior containment around the coater application roller and the flash-off area that is small in volume such that its temperature and relative humidity can be controlled more easily. This interior containment area is also used to collect solvent vapor for venting, destruction or recycling. This has an additional advantage to prevent people working inside the primary containment area from being subjected to elevated levels of solvent vapor. Such an environmental chamber system would have safety interlocks such that the tool could be stopped and any coating material safely contained if the solvent vapors approached flammability safety limits.

FIG. 17 shows a cross-sectional schematic view of one embodiment of a curing apparatus and method for skin-cure. In this apparatus, an air-knife (180) directs heated air on to the surface of a substrate (181) presented to the air-knife by a feed-in conveyor (182) and extracted by a feed-out conveyor (183). The air may be heated by an electrical element (184), as shown in FIG. 17, may also be heated by any other method known in the art. The air may be heated to any temperature useful in the method, such as to a temperature of 300° C. to 1000° C. Air may be forced through the heating element and air-knife by a fan (185). The temperature of the air is controlled by an electronic controller (186) and temperature sensor (188) located in the heated air stream. Optionally, overheat protection of the heating element may be provided by the electronic controller and, optionally, a second temperature sensor (187) located close to the heating element. When no substrate is present, air may flow from the fan through the heating element, through the air-knife and then directly to the exhaust (197). When a substrate is present, the air flows along the top surface of the substrate. In an embodiment, a pre-heating stage (189), for example an infra-red emitter, heats the substrate prior to the air-knife. The pre-heat temperature is controlled by an electronic controller (190) and a temperature sensor (191) with an optional safety over-heat sensor (192). In another embodiment, a flat plate attached to the leading edge of the air-knife forms a pre-heat chamber (189) with the top surface of substrate. This pre-heat chamber traps the hot air close to the substrate surface for a longer period allowing the hot air more time to pre-heat the substrate surface. A post-heating stage (193), for example an infra-red emitter (190) located subsequent to the air-knife provides additional heat that can extend the time that the substrate stays at an elevated temperature. The post-heating temperature is controlled by an electronic controller (194) and a temperature sensor (195), with an optional safety over-heat sensor (196). In another embodiment, there is a heating element in place of the pre-heat chamber. The pre-heating of the substrate can serve to reduce thermal stress during the very rapid heating under the air-knife and to provide an additional control on the peak temperature the substrate reaches under the air-knife, the peak temperature being a function of the initial temperature plus the temperature rise due to the air-knife.

A major advantage of this embodiment of a skin-cure system is that it allows the curing of a thin-film sol-gel coating without heating the entire substrate to a high temperature. A properly configured air-knife is able to heat the surface very fast (high power) without imparting a great deal of heat (energy) to the full substrate. Thus while the surface heats rapidly to a high temperature the overall substrate does not heat up excessively. In one embodiment the substrate is glass coated on one side with thin-film solar cells, and the opposing side of the glass is the desired surface for the sol coating. In this case, it is desirable to avoid heating and raising the temperature of the semiconductor photovoltaic material as much as possible while curing the sol coating. Thin-film solar materials such as CdTe, CIGS or amorphous silicon can be quite sensitive to elevated temperatures. High temperatures can cause dopants within the material to defuse in a detrimental manner or can cause metal electrode materials to defuse into the photovoltaic material. In some embodiments, the temperature of the photovoltaic cell may be kept from exceeding 100° C. to 120° C. as the sol is cured. Additionally, polymer materials within the finished solar module such as encapsulates may be kept from exceeding their glass transition temperature of 150° C. to 200° C.

FIG. 10 shows an example temperature profile for a skin-cure system. In this example the substrate is a dummy thin-film solar module consisting of two pieces of glass typical of those used in thin-film module manufacturing, laminated together with temperature sensors embedded between the glass sheets such that they measure the interior temperature of the dummy module and temperature sensors attached to the top surface. The module was moved at a speed of 1 cm/s under an air-knife set to an exit air temperature of approximately 650° C. and a gap distance (from substrate top surface to the air-knife opening) of approximately 1 cm. Two temperatures are shown, the top surface temperature representing the temperature reached by the interior of the dummy module. In this example the pre-heat chamber embodiment was used. From the profile, the pre-heat chamber caused an initial rise in temperature of the top surface (202) to approximately 100° C., there after the air-knife caused a very rapid temperature rise (200) to approximately 300° C. after which the post-heat infra-red emitter set to a temperature of 300° C. as measured by a sensor placed between the substrate and the IR emitter, maintains the top surface temperature (201) at approximately 200° C. Through-out the process the interior temperature never exceeds approximately 90° C.

In one embodiment, the substrate is glass of thickness 1 mm to 4 mm. In an embodiment of a skin-cure apparatus, the air-temperature exiting the air knife is between 500° C. to 750° C. as controlled by the power setting of the heating element and the volume of air provided by the fan. The speed of the substrate is between 0.25 cm/s and 3.5 cm/s. The resulting temperature of the substrate surface is between 150° C. to 600° C. and this temperature is attained between the start of the pre-heat chamber and the end of the air-knife. In other embodiments the substrate is pre-heated by an infra-red emitter to approximately 25° C. to 200° C. prior to the air-knife wherein it is further heated to approximately 150° C. to 600° C. Thereafter, the substrate is maintained at a temperature of between 120° C. to 400° C. until the end of the post-heat section. Such a configuration of the skin-cure apparatus has been shown to cure the sol coating while leaving the opposing surface at a temperature below 120° C.

The process of rapidly heating the substrate using the air-knife and then maintaining that temperature with radiant heat facilitates the curing of the sol-gel material. In an embodiment, the curing is achieved by providing sufficient energy so that a sufficient portion of the remaining Si—OH moieties within the coating undergo a condensation reaction and form Si—O—Si crosslinks that greatly strengthen the material enabling it to pass Taber abrasion testing to standard EN1096.2 with no more than 0.5% loss of absolute transmission. In other embodiments, the curing temperature is used to facilitate other processes such as volatizing a sacrificial component of the coating to form a desired porosity or a desired surface morphology. Other embodiments may use very high temperatures to completely oxidize all organic components in the coating creating a hydrophilic pure silica film. Yet further embodiments may use the heat and/or reactive gas composition of the air-knife to initiate chemical reactions that modify the properties of the coating, such as for example, surface energy, color, refractive index, surface morphology and surface chemistry. In embodiments, the skin-cure process works in concert with the composition and properties of the coating material to facilitate tuning of the properties of the final thin-film coating.

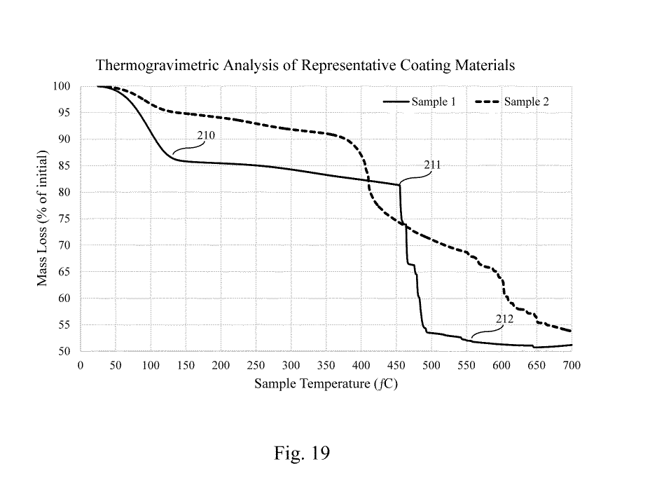

FIG. 19 shows a thermogravimetric analysis of representative samples of coating material. Thermogravimetric analysis is performed by heating a sample gradually and recording the loss of mass as various components of the sample volatize. When performed on coating materials such as these example sol-gel coatings for glass, it can be used to determine critical temperatures required to cure the coating material. The figure shows three temperatures of interest. Using Sample 1 from example 3 in FIG. 19 as an illustrative example, there is a point of inflection (210) at approximately 125° C., another much steeper point of inflection (211) at approximately 450° C. finally there is a flattening out (212) above 500° C. Without being bound by theory, these three points are interpreted as follows. As temperature increases to point 210 any residual water and solvent is volatilized and all easily accessible Si—OH moieties react, condense and release water. This represents a cured film that has attained a useful hardness and abrasion resistance at a relatively low temperature. Further heating in the range from point 210 until point 211 represents an approximately linear reduction in mass as additional remaining Si—OH moieties condense and release water. This temperature range represents increasing hardness and abrasion resistance of the material with increasing temperature, without detrimental effects on the coating. This reduction in mass causes a corresponding decrease in density and hence a decrease in refractive index. In coating materials that form hydrophobic films, the reduction in Si—OH will also result in an increase of the hydrophobic effect as measured by increasing water contact angle. Heating beyond point 211 begins to oxidize organic moieties within the coating and decomposes the material, the byproducts of which may then volatilize. In some embodiments these moieties may be methyl groups or other hydro-carbon groups or fluoro-carbon chains or any combination thereof. Other reactions may also occur such as for example the formation of SiC and SiₓO_{y}C_{z}. This temperature regime may be generalized as the oxidation of the organic components of the coating, reactions between byproducts of that oxidation with each other and with components of the film itself and the transformation of the coating to a substantially inorganic silica coating. At this point further heating no longer causes significant mass loss and the curve flattens out as indicated by point 212. Sample 2 of example 2 exhibits approximately the same shape and inflection points as Sample 1. It also illustrates that when more complex organic moieties are present in the coating the transformation that occurs after the second inflection point can be more complex and more prolonged. Therefore for the purposes of developing a process for curing these coatings we can determine from this analysis that a first low temperature cure can be accomplished at a temperature of approximately 125° C., which is the first point of inflection. A second higher temperature cure at the second point of inflection (approximately 450° C. for the material in Sample 1 and 350° C. for the material in Sample 2) results in increased hardness, abrasion resistance and hydrophobicity. Temperatures beyond the second inflection point result in the breakdown and modification of organic moieties that may in some embodiments be useful.

The curing process parameters including substrate speed, air knife output air temperature, air knife air flow volume, air knife opening distance to substrate surface, pre and post heating set temperatures are used to control process cure parameters including maximum temperature, rate of heating, duration at temperature, cumulative temperature exposure and rate of cooling that can be used to tune specific properties of the final cured film. One property is hardness as measured by nanoindentation methods. In some embodiments, the curing system described herein may cure sol-gel coatings on glass substrates to a hardness of approximately 0.2 GPa to 10 GPa and preferably to a hardness of approximately 2 GPa to 4 GPa. Another property is abrasion resistance. In some embodiments, the curing system described herein may cure sol-gel coatings on glass substrates to an abrasion resistance whereby they lose no more than 1% of absolute optical transmission as measured by spectrophotometer after 500 strokes of an abrasion test performed in accordance with specification EN1096.2 and preferably no more than 0.5% loss of absolute optical transmission after 1000 strokes. Such a test can be performed using a Taber reciprocating abrader model 5900 with a ratcheting arm assembly. A third property is surface energy as measured by water contact angle (WCA). In some embodiments the curing system described herein may cure sol-gel coatings to a WCA of approximately 60° to 120° and preferably to a WCA of approximately 70° to 100°. In other embodiments the film can be cured to a WCA of approximately 5° to 30° and preferably a WCA of approximately 10° to 20°. A fourth property is refractive index (RI) as measured by ellipsometer. In some embodiments curing system described herein may cure sol-gel coatings to a RI of approximately 1.25 to 1.45 and preferably a RI of approximately 1.35 to 1.42. A fifth property is final film thickness as measured by ellipsometer. The final film thickness is a function of the initial (pre-cure) dry film thickness and the cure parameters such that the cure parameters modify the initial dry thickness. In some embodiments the curing system described herein may cure sol-gel coatings to a thickness of 50 nm to 150 nm and to a preferred thickness of 70 nm to 130 nm.

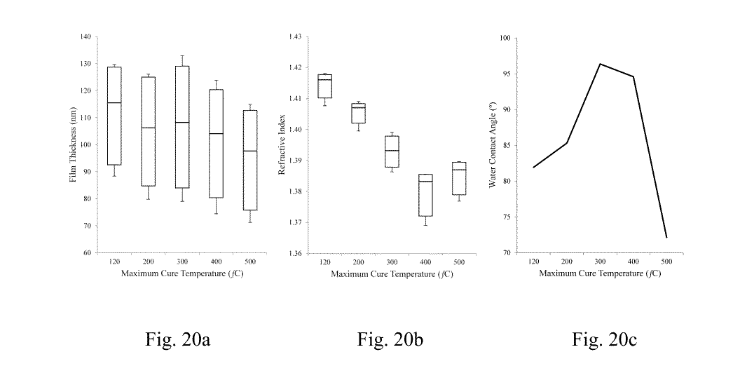

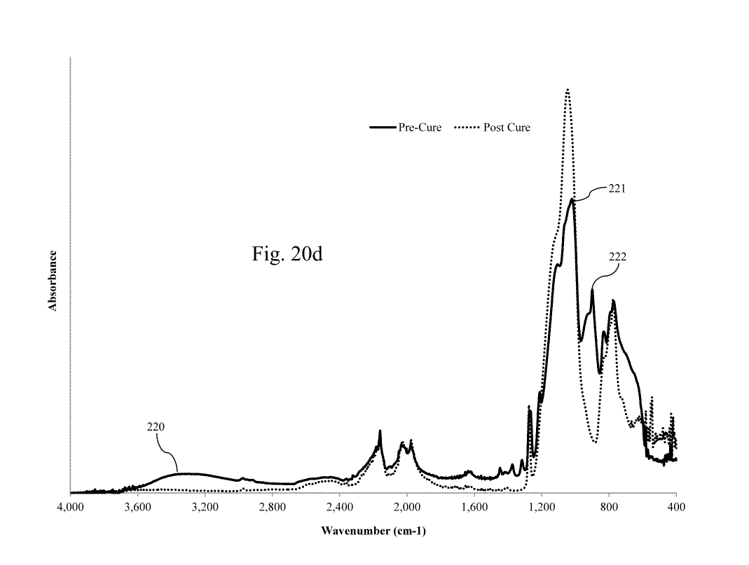

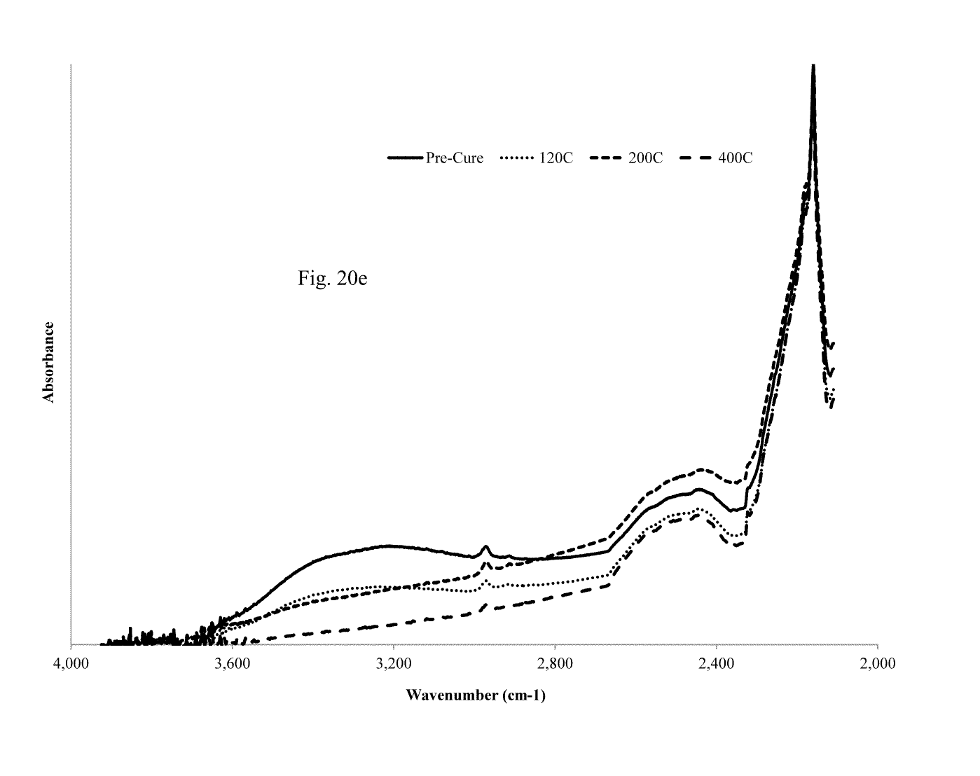

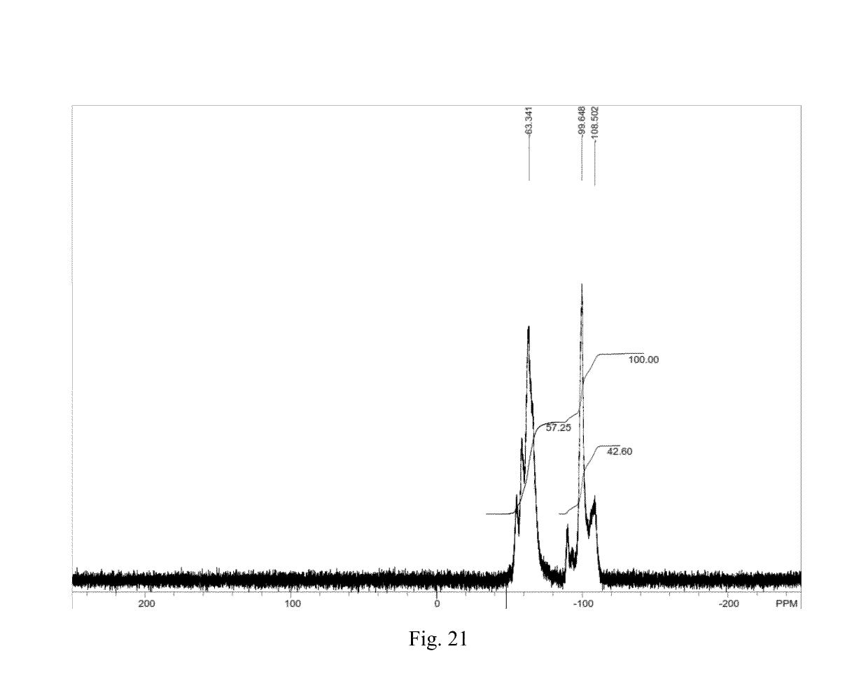

FIGS. 20a, 20b and 20c depict data for an exemplary sol-gel coating that demonstrate control of final film thickness, refractive index and water contact angle as a function of maximum cure temperature.

FIG. 20d shows Fourier transform infrared spectra (FT-IR) of sol-gel coating material from example 3 taken before and after a cure process step. This analysis technique shows how chemical bonds within the material change during the curing process. In particular the spectral peaks denoted by points 220, 221 & 222 have changed during the process. Without being bound by theory, these changes can be interpreted as the reduction of Si—OH bonds through condensation causing the reduction of the peaks at points 220 and 222. These bonds are converted to Si—O—Si bonds causing the increase in the peak at point 221. This analysis technique can be used to quantify the proportion of Si—OH bonds that condense and hence to quantify the degree to which the film is cured. FIG. 20e shows FT-IR spectra of example 3 cured at different cure temperatures of 120° C., 200° C. and 400° C. FIG. 20F shows FT-IR spectra of example 2 before and after cure.

The coating and curing process steps may further be configured to create coatings of varying complexity and structure. In embodiments, any combination of coating technique and curing technique may be used to achieve a final coating for a substrate. Embodiments of such combinations may include coating via a flow coating technique followed by a skin cure process or cure by conventional means, coating via a roll coating technique followed by a skin cure process or cure by conventional means, and the like. To generate multilayer coatings, any combination of coating and curing apparatus may be used sequentially to generate such a coating. The sequential use of such apparatus may be enabled by an arrangement that places multiple coating apparatus and curing apparatus in sequence. Alternatively, handling facilities may exist for handling the substrate between one or more coating and curing apparatus. For example, two roll-coaters may be placed in sequence with an optional flash-off heater in between. This facilitates coating of a first layer by the first roll-coater, drying of the layer by the flash-off station, then deposition of a second layer by the second roll-coater before curing in a skin-cure station or in a simple oven. Alternatively, a high temperature skin-cure step may be interposed between the roll-coaters to enable a high temperature heat treatment to the first layer before application of the second layer. It is understood that this technique for multiple layer coatings may be extended to more than two layers. Multi-layer coatings manufactured by this technique may be high performance anti-reflective interference type coatings or multiple layers coatings could be used to modify the surface energy of the top surface coating by for example adding a fluorinated silane mono-layer to an underlying layer to make the final coating hydrophobic and oleophobic on the environmentally exposed surface. The multi-layer coatings may be used to enhance single layer anti-reflective coatings by adding a lower refractive index material on the environmentally exposed surface to create a graded index coating between the environment and the underlying substrate. In embodiments, a second layer of coating may be applied to an existing base layer to provide a functional benefit of the multi-layer coating in combination with the base layer. For example, a mobile phone/touch screen glass may be coated with an inorganic coating that provides anti-scratch benefits, then a low-temperature anti-soiling coating may be on top of the anti-scratch coating.

The foregoing apparatus and methods are particularly well suited to the application of sol-gel thin-films to glass. In an embodiment, the glass to be coated is the front (sun facing) surface of a solar module and the sol-gel thin-film is an anti-reflective coating. Either bare glass may be coated and/or cured by the apparatus or fully assembled solar modules or solar modules at any intermediate stage of manufacture. In other embodiments, the apparatus may be used to coat and/or cure windows, architectural glass, displays, lenses, mirrors or other electronic devices.

In an aspect, a coating and curing apparatus may include a conveyor system of a combination roll coating and curing facility, wherein the combination roll coating and curing facility comprises at least one roll coating facility and at least one curing facility, and wherein the conveyor system is adapted to transport a substantially flat substrate through the combination roll coating and curing facility, a processor that controls a process parameter of the at least one roll coating facility, and an air knife of the at least one curing facility, wherein the air knife is adapted to direct heated air to a portion of the flat substrate as it is transported through the at least one curing facility, wherein the at least one roll coating facility is adapted to coat the substantially flat substrate with a sol gel coating material. The substantially flat substrate may be a part of at least a partially finished solar module. The apparatus may further include an electrical element disposed within the air stream to heat the air flowing through the air knife. The air may be heated to a temperature between about 300° C. and 1000° C. The apparatus may further include a fan in the air stream that directs air to the air-knife. The apparatus may further include an electronic controller that controls the temperature based on readings from at least one temperature sensor located in the air stream. The apparatus may further include an exhaust to remove heated air from the apparatus. The apparatus may further include a flat plate attached to the leading edge of the air-knife, wherein the flat plate is adapted to form a pre-heat chamber with the top surface of the substantially flat substrate. The apparatus may further include an infra-red emitter disposed along the conveyor system prior to the air knife, wherein the infra-red emitter is adapted to heat the substantially flat substrate to a temperature of between 25° C. to 200° C. The apparatus may further include an infra-red emitter disposed along the conveyor system subsequent to the air knife, wherein the infra-red emitter is adapted to maintain the flat substrate at a temperature of between 120° C. to 400° C. The process parameters may include at least one of a doctor roller spacing and/or pressure to an application roller, the application roller spacing or pressure taken with respect to the substantially flat substrate, a speed at which the substantially flat substrate is conveyed by the conveyor system, and in the case of reverse roll-coating, a difference in speed between the substantially flat substrate and the application surface of the application roller. The processor may further control a process parameter of the curing facility. A plurality of roll coating facilities and curing facilities may be arranged sequentially. The air-temperature exiting the air knife may be between 500° C. to 750° C. The speed of the substantially flat substrate on the conveyor system may be between 0.25 cm/s and 3.5 cm/s. The resulting temperature of a surface of the substantially flat substrate may be between 150° C. to 600° C.

In an aspect, a method of coating and curing may include conveying a substantially flat substrate to be coated with a conveyor system through a combination roll coating and curing facility, wherein the combination roll coating and curing facility comprises at least one roll coating facility and at least one curing facility, roll coating the substantially flat substrate with a sol gel coating material with the at least one roll coating facility, and curing the sol gel coating material on the substantially flat substrate with an air knife of the at least one curing facility, wherein the air knife is adapted to direct heated air to a portion of the substantially flat substrate as it is transported through the curing facility by the conveyor system. A sol-gel coated substantially flat substrate may be formed by the method, wherein a portion of the sol-gel coating material is cured while a different portion of the sol-gel coating material remains uncured.

In an aspect, a method of tuning the performance of a sol gel coating may include determining a desired cure temperature profile to achieve a specific performance metric for a sol gel coating using at least one physical analysis method, selecting settings for an air knife curing system's operating parameters to achieve the desired temperature profiles for the sol gel coating on a substantially flat substrate, and curing the sol-gel coating on the substantially flat substrate with the air knife curing system. The at least one physical analysis method may include at least one of thermogravimetric analysis, Fourier transform infrared spectroscopy, ellipsometry, nanoindentation, abrasion testing, spectrophotometry, and a water contact angle measurement. The air knife curing system operating parameters may include at least one of substrate speed, air knife air-flow volume, air knife output air temperature, air knife opening distance to substrate surface, a temperature set-point for a pre-heating zone and a temperature set point for a post heating zone. The performance metric for the sol-gel coating may include at least one of hardness, abrasion resistance, surface energy, refractive index, optical transmission, thickness and porosity. The method may further include a step of coating the substantially flat substrate with the sol gel coating using a roll-coating system before the step of curing. A sol-gel coated substantially flat substrate may be formed by the method. The specific performance metric may include a hardness of the sol-gel coating within a range of 0.2 GPa to 10 GPa. The specific performance metric may include a test in which no more than 1% of absolute optical transmission is lost after at least 500 strokes of an abrasion test performed in accordance with specification EN1096.2. The specific performance metric may include a water contact angle where the water contact angle is within 60° to 120°. The specific performance metric may include a water contact angle where the water contact angle is within 5° to 30°. The specific performance metric may include a refractive index of the cured, coated sol gel from 1.25 to 1.45. The thickness may be approximately 50 nm to 150 nm. A sacrificial component of the sol-gel coating may be volatilized to form a desired porosity.

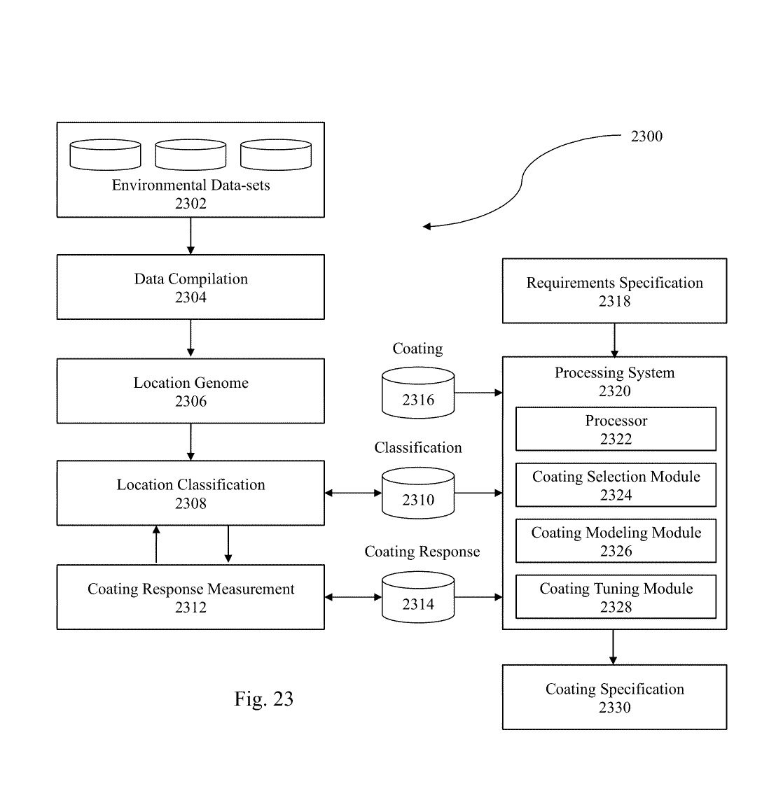

Methods and systems for determining prescriptive glass coatings for achieving advantageous interactions with the environment for solar modules and for other applications are described. FIG. 23 depicts a platform 2300 that prescribes a custom coating for glass that will be applied to the glass that is based upon the environmental factors that exist at the location where the glass will be used. In some embodiments, the glass is the cover glass for a solar module.

The platform 2300 depicted in FIG. 23 may be integrated with a solar module manufacturing facility, a glass manufacturing facility, and the like. In embodiments, the platform may be integrated with a pricing/vending system in order to accurately price coatings based on demand and the cost of starting materials/reactants, as well as to accurately price finished products after coating.

The system may include a processing system 2320 with a processor 2322 that ingests various data and inputs from the Requirements Specification 2318 and the various databases 2310, 2314 and 2316, and is operative to output a coating specification that includes a coating material, a coating process, a curing process and the like. The processing system 2320 may include various modules that support the selection process. The modules may be based on algorithms that employ one or more rules to make the selection based on the data and/or inputs. Some modules, which are more fully described herein, include a selection module 2324, a modeling module 2326, a tuning module 2328, and the like. Operation of the modules is further described herein.

In the first step 2302, geo-located environmental data from many sources is gathered. This data may include basic climatic classification data such as Köppen-Geiger, Trewartha & Holdridge Life-Zone; meteorological data sets such as those provided by NOAA in their Climate Data Online (CDO) datasets, NREL's National Solar Resource Database (NSRDB) and their Typical Meteorological Year dataset and the UK's UKCP09 dataset; pollution data such as the EPA's Air Facility System (AFS) & Air Quality System (AQS) and the European Environmental Agency's Long-Range Transboundary Air Pollution (LTRAP) dataset; Soil classification datasets such as Pennsylvania State University's CONUS-SOIL and STATGO databases and the Harmonized World Soil Database (HWSD) from the International Institute for Applied Systems Analysis (IIASA) and biological datasets such as annual pollen counts and the like. Data such as measurements of temperature, precipitation, wind, rainfall, snowpack, river flow, time spent below dew point, solar, spectral distribution, air quality index, specific pollutant concentration, SO₂ concentration, dust type/concentration/dust chemistry, alkali lakebed presence, road salt exposure, iron oxide exposure from train tracks, petrochemical/combustion exposure from nearby industry, tire/break debris from nearby roads, seabird population, insect populations, biofilm/mold prone areas, moss, altitude, rivers, lakes, desert, seaside, sea foam exposure, and the like from diverse sources may also be included.

In the second step 2304, Data Compilation, these data sets are analyzed using a Geospatial Information System (GIS) tool such as for example ArcGIS available from Esri. This analysis can consist of importing each dataset as a set of maps, for example maps of annual rainfall or night-time dew-points or soil type. These maps can then be viewed and manipulated as a stack of layers. New layers can be derived by algorithmically combining multiple layers. For example, in some embodiments maps could be derived that were a combination of high temperature, high humidity, sandy soil, windy conditions and desert climate zone that would show areas that may have a high probability of soiling. Using these tools it is possible to associate many different variables derived from a diverse set of environmental datasets with specific geographic locations in a single GIS database. This database then enables creation of a ‘location genome’ in step 2306. This is a set of values for particular variables associated with specific locations that describe many of the relevant data for predicting the interactions of glass surfaces with that environment. For example in some embodiments this genome includes values for daytime relative humidity; night time dew-point, prevailing wind direction, upwind soil type, annual pollution of PM10 particles, annual pollen counts, annual rainfall total, rainfall frequency and other relevant variables.

Derivation of the location genome can be optimized in several ways. In one embodiment, locations can first be filtered to include only locations that are suitable for the installation of solar energy generation systems. For example, locations with steep terrain or slopes, locations occupied by rivers, lakes & forest, locations of ecological, archeological or geological sensitivity, locations occupied by roads, highways and freeways could be excluded. Additionally locations could be ranked according to how suitable they are for the installation of solar energy generation systems. For example, locations with higher than average electricity consumption, locations with above average solar resources (insolation), locations close to electrical grid transmission lines or locations that enjoy regulatory, governmental or financial incentives that support solar energy generation could contribute to a higher ranking Locations could be evaluated in the order in which they were ranked. Another optimization is the size of the locations evaluated, for example locations might be 1 hectare, 1 square kilometer, or many 10's of square kilometers. Additionally the location size might be varied depending on other database variables, for example, smaller location sizes might be derived for urban environments where small-scale variables such as proximity to roads or pollution sources such as industrial sites might be relevant. Larger location sizes might be used in remote or rural areas where in comparison the environment is homogenous over larger areas.

In step 2308 locations are sorted into a limited number of classification groups based upon their genome values. The process of sorting classification groups locations is done in a manner that is functionally relevant to the interactions of glass with the environment at that those locations. That is to say, glass placed at locations that share the same classification experience similar environmental interactions with respect to phenomena such as soiling, cleaning and biological interactions. Thus classifications are useful because they serve to reduce the very large number of unique location genomes to a smaller number of classes that can then be used to select specific coating properties.

Development of different classifications can be by theory, based upon empirical evidence from field sensors or lab experiments or a combination of all or some of these. In some embodiments, theory may include concepts of soiling mechanisms such as wind deposition of dust where the rate is a function of mean dust sizes and mean wind speeds; deposition rates as a function of episodic dust events that are non-linear with respect to time; natural cleaning is a function of wind speed and adhesion forces of dust to glass; cleaning as a function of dew formation and cleaning as a function of rainfall amounts. Field sensors may include existing infrastructure such as solar energy facilities, buildings and glasshouses where the interaction of glass with the environment can be measured; or dedicated instruments designed to measure interactions such as soiling, bio-fouling, reflection and transmission of glass in a particular location; or sensors designed to measure meteorological phenomena and/or air pollution or particulates. Lab experiments may include data on controlled deposition of dust onto glass; or growth of bio-films on glass; or cleaning of glass by simulated natural processes.

Classifications could be hierarchical, such that high-level general classifications such as for example “hot” may have sub-classes such as for example “hot-humid” and “hot-dry”. There may be multiple levels of sub-class. It should be understood that where terms such as “hot” or “humid” are used, they represent specific ranges of values that are defined numerically.

Classifications may broadly follow climate classifications given that climate and associated weather is a primary variable affecting the performance of glass. However, climate classifications are refined by the addition of other data mentioned above to indicate different interactions of glass even within the same climate classifications or indeed interactions of glass that are similar even across different climate classifications. Thus, in some embodiments, the classification process may start with climate variables such as “hot” and “dry”, then add environmental variables such as land use: “urban”, “agricultural” or “industrial” and pollution “high” or “low”.

The possible set of classifications is not static. It can evolve by changes to existing classes and by the addition or removal of classes based upon new data about glass performance in particular environments or new learning about environmental factors that were not previously taken into account.

Location classifications are stored in a database 2310 such that a query for a location designated by latitude/longitude or zip/postal code or city name or county name or the like can retrieve the correct classification assigned to that location.

In step 2312 the response of glass with and without various coatings is tested at locations with selected classifications. This step serves two functions: as mentioned above measuring the performance of glass at specific locations verifies the classification assigned to that location and helps to refine the classification system in general; second, the differences in response by different coatings to a particular class of environment enables building a coating response database 2314. Several response variables of glass can be measured. Some embodiments include measurements of light transmission and reflection; soiling rate; self-cleaning performance; anti-fouling performance; durability; resistance to abrasive factors including windborne sand and cleaning by human or machine; degradation due to environmental factors such as UV light exposure, high & low temperatures, rain, hail and snow.

Coating response can be measured by both manual and automated methods. In some embodiments specialized test stations are deployed that measure optical transmittance by placing an optical sensor such as for example a calibrated solar cell behind a sample piece of coated glass and continuously measure light intensity. A test station may consist of a plurality of such sensors testing different coated glass samples. Additionally the test station may include sensors for directly measuring solar irradiance and weather factors such as temperature, dew-point, humidity, wind speed and direction and the like. The station may also include the means to record the output of all sensors digitally, to store that data locally and to transmit that data electronically to remote computer servers for storage and analysis. The station may also include a means for keeping reference pieces of coated glass in a clean condition. Additionally or alternatively coated glass samples may be manually inspected and measured on a periodic basis. For example a technician could assess soiling by measuring the reflectance using a portable spectrophotometer from a sample of coated glass, or take photographs for later image analysis of surface contamination; or take samples such as swabs for biological or chemical analysis; cleaning tests could be performed to assess the relative ease or difficulty of cleaning different coated glass samples.

Coating response might also be measured based on performance of one or more coated solar modules in a full solar energy generation system. The response could be measured relatively between differently coated solar modules or relative to predictive models of solar module performance.

There are several possible response variables that may be measured for each different coating of many different coatings may be tested at a particular location. Several locations per class may be selected for testing, and multiple different classes of coatings may be tested. Additionally responses may be measured over the course of significant time in order to measure seasonal and annual variation. All these data are stored in a coating response database 2314.

The requirements specification 2318 is a set of requirements typically provided by a coating customer who wishes to purchase the optimal coating material for a particular application. The specification includes the geographic location (latitude/longitude, address, zip code, city name or county name and the like) where the coated glass will be used; the type of use, for example ground mounted or roof mounted or building integrated or heliostat and the like; the orientation (altitude & azimuth) of the coated glass such as flat, angled, vertical, facing west and the like; the ranked performance criteria for the coating for example, durability, anti-soiling performance and optical performance; economic requirements such as a cost limits; manufacturing integration requirements such as limits to coating curing temperature or a requirement to coat substantially completed solar modules or to coat bare glass;

In embodiments, the requirements specification 2318 may include data on various aspects of the solar glass or solar module. Information on the surface of the glass, such as a front surface, may include the type of glass (e.g. float glass or patterned glass; smooth or textured), the chemical composition (various types borosilicate, soda lime, etc.), the presence of a tin coating, any aesthetic elements such as colors or patterns, and the like.

In embodiments elements within the requirements specification 2318 can guide operation of the processing system 2320 with specified rules. For example, if the requirements specifications 2318 indicate that there is tin on the surface of the glass and the input specification is that the glass should be a particular color, only certain pigment additives to the coating might be suggested by the processing system 2320. In embodiments, if the requirements specification 2318 is that the glass is textured, the processing system 2320 may only suggest coatings that can be applied with a coating technique that works well on the surface, such as flow coating or roll coating. If the requirements specification 2318 is that the glass surface is rough, the processing system 2320 may only suggest coatings that can be applied with a coating technique that works well on the surface, such as spraying. The coating may comprise lower volatility solvents. If the requirements specification 2318 is that the glass is not flat, the processing system 2320 may only suggest coatings that can be applied with a coating technique that works well on the shape of the glass, such as spraying. If the requirements specification 2318 is that certain optical elements are present, such as a lens, diffuser, mirror, and the like, the processing system 2320 may only suggest coatings that can be applied with a coating technique that can accommodate the presence of these elements. If the requirements specification 2318 indicates a bulk property of the glass, such as low refractive index, the processing system 2320 may only suggest coatings with a refractive index less than the glass' in order to obtain an anti-reflective property. In embodiments, the processing system 2320 may calculate the optimal refractive index indicated by the requirements specification 2318 and only suggest coatings that meet that calculation. For example, a transparent material with a high refractive index, such as sapphire with a refractive index of 1.77, might be coated with a coating having a refractive index of 1.33. If the requirements specification 2318 indicates a presence of certain mechanical components of the solar module (e.g. junction box, rails, brackets, etc.) or a certain shape of the glass, the processing system 2320 may select a coating technique that can accommodate the components, such as selecting flow coating versus roll coating, for example.

Information relating to a surface of the glass, such as a back surface, may include information about any encapsulant material present, such as EVA (ethylene vinyl acetate) or PVB (polyvinyl butyral); any electrode material applied such as TCO (transparent conducting oxide) or any applied semiconductor material such as CIGS (copper indium gallium selenide). For example, different semiconductors may have different sensitivities to the solar spectrum, so the coating material may be selected to tune the light transmission through the solar module. In an embodiment, the coating may be selected to filter certain wavelength, such as to allow light at working wavelengths but reducing light at other wavelengths, such as in order to increase the longevity of the back surface material that may be degraded over time by incident light. For example, the selected coating may contain UV-sensitive dyes, rear earth elements such as cerium, quantum dots or other dopants that absorb photons in the UV range, or other material that absorbs UV photons and either emits the energy in a more useful or less damaging portion of the spectrum or dissipates it through thermal loss. Information relating to a surface material may be used to select a curing technique or process that does not adversely affect the material. For example, if TCO is heated, its resistivity may be altered. In another example, if there is a semi-conductor junction on the back surface of the glass and it is heated, the materials, such as any dopants, may move and unwanted intermetallics may form. Semi-conductor junctions may be manufactured with a thermal budget that should not be exceeded, so such information in the module specifications 2168 may be used to select a curing temperature. In embodiments, coatings may be designed or selected to fit the thermal profile of the material on the other side of the glass.

The requirements specification 2318 may also include variables that may be used as filters applied to various options and/or data available to the processing system 2320. These variables may include optical factors, biological factors, weather factors, soiling factors, cleaning factors, geospatial factors, user requirements, and the like. Process or in-process specifications may be a variable. For example, the manufacturing stage may be an important variable to consider, such as whether the solar module is substantially complete, whether the solar glass is to be coated before placement in a module, whether the solar module is in a stage that is not substantially complete, whether the solar module is installed at an end-use location, and the like. Certain coatings may not be suitable for an in situ application for example. Other process variables may include temperature restrictions, other coatings to be applied during manufacture, the solar module assembly equipment in use, volume output of solar modules required, and the like. Other variables may include supply chain simplification, economic factors, ability to retrofit, coating techniques and other customer specifications or requests, such as glass type, anti-soiling properties, anti-reflective properties, abrasion resistance, spectral filtering properties, spectral shifting properties, aesthetics/color, system integration requirements, desired coating equipment, extent or type of available personnel to coat or reapply coating, ambient temperature around an installed module receiving a coating, environmental factors, the presence, nature and extent of any existing coatings, and the like.

In embodiments, the requirements specification 2318 may include supply chain simplification as a variable such that it may refer to the need to reduce the inventory of an item required, the need to use items already in stock, or the desire to reduce the need for expensive components. Economic factors may refer to overall cost of a project, market price of certain chemicals (such as constituent materials for coatings), prices for coatings, prices of energy produced by a module, predicted energy prices for a region, budgetary constraints or requirements, the time value placed on money (e.g., comparing current expenditures with future savings), and the like. For example, if a party places a high premium on long-term optimal performance and is relatively insensitive to the time value of money, the party may prefer a coating that provides optimal long term energy performance (e.g., has high anti-reflective properties and is very durable) even if the coating is more expensive to purchase or to apply. Meanwhile, a party with a short-term economic focus may prefer a less expensive coating that provides good enough performance for a few years. In embodiments, the methods and systems disclosed herein may provide alternatives that present the economic tradeoffs among different coating options, such as providing side-by-side comparison of predicted price and performance for various coatings over different time periods.

In some embodiments the requirements specification 2318 is a data file such as for example a structured text file for example using a schema like XML or HTML, or a text file consisting of a list of attribute-value or name-value pairs, or a binary file containing a data structure, or a spreadsheet file or the like. In other embodiments requirements could be entered by a user into a user interface such as an electronic form, survey, questionnaire or a series of question-answer steps (“wizard”).

In some embodiments the process of gathering the requirements specification might be a standalone step that is performed “in the field” for example by a salesperson at a customer's place of business. It may be performed by a dedicated program on a mobile computing device or a computer terminal or a website displayed in a web browser. It may generate a file such as mentioned previously or it may directly transmit or upload the requirements to a server. In some embodiments it may be part of a larger interactive user interface that both gathers the requirements specification and reports results directly back to the user in real time.

A coating database 2316 stores performance, process and cost data for different coating materials. In some embodiments the performance data includes measurements of refractive index, surface energy, porosity, color, viscosity, hardness, modulus, chemical composition, toxicity, VOC content, safety data. Process data includes curing temperature profiles, wet to dry thickness relationship, coating conditions like acceptable temperature and humidity ranges, surface pre-treatment requirements. Cost data includes raw materials costs, material production costs by production location and cost of ownership for the full coating manufacturing step.

The coating selection module 2324 selects one or more coatings from the coating database 2316 that best matches the requirements specification 2318 based upon the information stored in the coating database 2316, the coating response database 2314 and the location classification database 2310. The selection is achieved by one or more algorithms performed by the coating selection module.

It should be appreciated that while the location classification database 2310, the coating response database 2314 and the coating database 2316 are discussed as separate entities, there is no need for this to be the case. All of these data-structures could be in a single database, or multiple databases or incorporated into other databases or be stored in other manners as needed or any combination thereof. The only requirement being that the data may be stored and retrieved.

In some embodiments the coating selection module 2324 evaluates the location given by the requirements specification 2314 and then retrieves its location classification from the location classification database 2310. It then retrieves the coating response data for that classification from the coating response database 2314. The retrieved coating responses may then be joined with the additional coating information from the coating database 2316 to generate a data view or table of everything known about the available coatings for the location of interest from the requirements specification 2318. The coating selection module may now seek a best match between the criteria in the requirements specification 2318 and this data view.

In some embodiments the coating selection module 2324 may use a two-step approach wherein the first step eliminates candidate coatings based upon performance, process or cost requirements before further evaluation in a second step. For example, the requirements specification 2318 may require that the coating cure temperature be below 300° C., so therefore all coatings that require higher curing temperatures may be eliminated from further consideration. In another example a hydrophobic coating might be required allowing the elimination of non-hydrophobic coatings from further consideration. In general the requirements specification 2318 can be used to pre-filter the coating candidates by implementing a set of “must-have” requirements that are evaluated first.

In some embodiments the requirements specification provides a list of ranked performance criteria, such that higher ranked criteria are evaluated first and only coatings that meet the higher ranked criteria are evaluated for each successively ranked criteria. For example the performance criteria might be ranked as 1) durability, 2) anti-soiling and 3) coating uniformity. In this case, only coatings that met the durability requirement would be evaluated for anti-soiling and only coatings that met both would be evaluated for coating uniformity. It should be recognized that performance criteria might be specified numerically such as performance >=x; or relative manner such as “high” meaning better than the population average or in the 75^{th} percentile or the like; or may be implicit, meaning that criteria should be maximized, for example maximum durability.

In some embodiments the requirements specification might provide a weighted list of performance criteria such that for each criteria the performance achieved by a coating is multiplied by the weight and a score for the coatings is calculated. Then using the calculated score a ranked list of candidate coatings is assembled wherein the highest ranked coatings best match the desired performance criteria.

In some embodiments, numerical performance data may be normalized, for example using logarithmic or linear scales to allow comparisons to be made between different variables and between selection criteria. Other numerical techniques may be used to compare data such as best fit or least squares to determine degree of similarity between data.

The selection module 2324 may be embodied as a mapping engine. The mapping engine may be programmed with various guesses or anecdotal material for selecting a coating based on a single link point.

The selection module 2324 may be embodied as a matrix. Each unique intersection of factors in the matrix may be mapped to a coating that is adapted for that combination of factors. The matrix may be 3×3, 4×4, M×N×O×P, and the like. Thus, when input is received, the matrix can be implemented to identify a suitable coating.

In some embodiments the coating selection module may be used interactively by a user such that lists of candidate coating materials with their associated response at the location of interest are presented and the user applies filters to the list to reduce the number of displayed coating materials and narrow the choice. For example several filters might be presented such as hardness, surface energy, reflectance, self-cleaning performance. For each filter a set of allowed values are displayed from which a value for the filter can be selected. When the user selects a filter value it is applied to the list, removing all list items that do not pass the filter criteria. Provision would also be made for sorting the displayed list by values so that the user could interactively rank the list by performance criteria of interest.

In some embodiments the user could select specific coating materials that have been identified by the coating selection module and request a price quotation or a pricing schedule versus volume or a draft or outline supply agreement.

In some embodiments the coating selection module can generate a configuration file or parameter file or data to be entered manually or interface directly through an API or the like, to solar energy generation system performance prediction software such as for example NREL's System Advisor Model (SAM) and the commercially available program PV_{SYST}, such that a user can simulate system performance with the coating material and hence compare system performance with and without the coating or between multiple different coating materials. Additionally, the coating selection module might be setup to work interactively with tools like SAM and PV_{SYST} to provide calculate data such as KWHr/KWp and LCOE for a particular system design input by a user so that they could compare the energy generation and financial performance of the target system with various coatings.

In an embodiment, the coating modeling module 2326 enables a process for developing a custom coating adapted to the conditions/soiling types that the solar/glass module is in or will be in, wherein the process includes using an algorithm to generate a custom coat formula based on information about the conditions/soiling types along with any other inputs. Upon generating the new coating, it may be deposited into the coating database 2316 for future use in a matching process. For example, a certain monomer known to form abrasion resistant sol gels may be combined with another monomer known to form corrosion-resistant sols. In some embodiments, a matrix of monomer properties may be used to form a starting point for experimentation in order to develop new sols suitable for a custom environment and/or input requirement. In some embodiments, the new coating formula may be generated by combining existing coatings from coating database 2316.

In an embodiment, the modeling module 2326 enables a determining a soiling fingerprint for a location and a coating adapted for the soiling fingerprint. Based on the weather conditions in a location, a soiling fingerprint may be determined. In embodiments, really heavy rain may clean glass while light rain may re-distribute dirt on glass. In other embodiments, light wind may transport dust to glass, while strong winds may clean glass. In yet other embodiments, certain levels of humidity and dew points may result in the formation of water on glass and may result in dirty glass. Patterns of these soiling and cleaning mechanisms of nature may be analyzed by the modeling module 2326 to determine a soiling fingerprint for a location. For example, the modeling module 2326 may generate a soiling fingerprint for a location that is humid in the morning, then sunny and windy in the afternoon. The modeling module 2326 will then identify a coating that is effective (e.g. mitigates soiling) with respect to the soiling fingerprint and those mechanisms for soiling inherent in the fingerprint. In other embodiments, such as where the soiling fingerprint indicates an overall cleaning mechanism for the glass, the modeling module 2326 may match a coating that enhances the overall cleaning mechanism. Over time, machine learning applied to the output of the modeling module 2326 may result in improved and more rapid selection of candidate coatings. In embodiments output from the modeling module 2326 may be further narrowed or filtered by any other module of the processing system.

In embodiments, the final number of coatings is selected by the modeling module 2326 may be greater than one, for example, a number of coatings selected may be in the teens. In order to narrow down the coatings for use in an area, a weighting function may be applied to the output of the module 2326. For example, relative humidity may be weighted heavier than wind speed for one location, but in another location, the user may choose to weight wind speed more heavily than relative humidity.

In general, tunability of the anti-reflective coatings may relate to real-time tunability, such as during a stage of the manufacture of a solar module, and/or pre-selected tunability, such as a customer or designer selecting from a menu of options or inputs available to generate a solar module tuned for a particular set of requirements. Of course, even if tunability options are pre-selected, they may also be modified in real-time during a manufacturing run.

In an embodiment, the tuning module 2328 may determine a modification to one or more of a custom coating, a coating technique, and a curing technique, wherein the process includes using an algorithm to determine one or more modifications that satisfies one or more input conditions. When input requirements are made for a certain property or properties of the coating, the tuning module 2328 may be queried to determine if there is some way of tuning an existing coating formulation, or an existing coating process in order to satisfy the input requirement. In other embodiments, the tuning module 2328 may be embodied as filters to the selection module 2324.

For example, if the requirements specification 2318 is to continue to use a particular batch of coating but based on a desire to modify module performance, such as to increase the transmission gain for a subset of solar modules by a particular percentage, the tuning module 2328 may calculate a modification in the thickness of the coating required to make the modification and subsequent solar modules will only be coated to that newly calculated thickness.

In another example, the thickness of the coating may be tuned based on a desire to modify module performance on a module-by-module basis if the input is that all of the modules manufactured should perform in a uniform fashion. Each module may have its performance tested and the tuning module 2328 may calculate a coating thickness required to obtain the same module performance as other coated solar modules. In an embodiment, the coating itself or an aspect of the coating process may be selected to modify the module performance. In one example, the coating or how the module is coated with the coating may be selected to increase the solar energy collection efficiency or otherwise improve its performance.

In another example, the coating or how the module is coated with the coating may be selected based on a desire to modify module performance such as to decrease the solar energy collection efficiency or otherwise decrease its performance. A manufacturer may have an inventory of only one kind of panel, so in order to match the power profile of existing solar modules, a coating may need to be applied to either decrease or increase the solar panel's performance. In embodiments, the solar modules may first be binned by performance and the coating may be serially applied to those modules that fall within a first performance bin. The custom coating or some aspect of the coating process is selected so that the solar modules in the first performance bin match the performance of solar modules in a second performance bin.

In embodiments, the tuning module 2328 may enable optimizing the peak performance of the solar modules by selecting a particular thickness of coating layer. For multi-layer coatings, the thickness of all of the coating layers may be tuned to obtain different peaks while maintaining control over interference effects between interfacial layers of a multi-layer coating.

In embodiments, the tuning module 2328 may enable tuning a coating to accommodate variability in peak performance of solar glass/modules based on, for example, some inconsistent manufacturing factor. For example, solar module manufacturers may purchase lower cost solar materials or materials of a specific output wattage and use the tuning module 2328 to make coating selections that enable obtaining higher performance out of cheaper solar cells or that enable obtaining different output wattage than originally specified. In embodiments, the coating may be optimized on a per module basis, to accommodate this variability or any other variability, by modifying an aspect of the coating or the coating process by using the real-time, in-process control enabled by the system.

In an embodiment, a post-coating curing technique may be selected by the tuning module 2328 in order to adjust an aspect of the coating. For example, the curing temperature of individual modules or batches of modules may be modified in order to modify the density, porosity, or hardness of the coating.

In an embodiment, the tuning module 2328 may specify addition of an activatable chemical agent so that certain properties may be obtained if so desired. A heat-activated agent may be added to the sol material before coating. For example, a porogen may be added to the coating material that is only active at high temperature, wherein the material evaporates at high temperature leaving pores. In another example, a cross-linking agent may be added, wherein cross-linking occurs above a certain temperature to obtain a stronger coating material. In both these examples, the coating material may only be modified if and when a threshold temperature is applied. In yet another example, the activatable chemical agent may be a color-changing agent, such as a thermochromic agent. As the temperature of processing the coating changes, the color of the material may change. For example, the agent may be activatable with application of a heat gun or air knife. In embodiments, the temperature at which the coating is cured may determine a final color of the coating. In embodiments, certain agents may be activated with UV exposure.

In an embodiment, the tuning module 2328 may specify an elevation in curing temperature sufficient to render the cured coating hydrophilic.

In an embodiment, the tuning module 2328 may specify addition of a material that undergoes a phase change. In an embodiment, a crystalline material may be included in the coating, wherein the crystalline material undergoes a phase change with a modification of a condition. For example, if the input is a desire for an aesthetic change, the crystalline material may cause a change in color or transparency of the coating based on a phase change. Such a property may be desirable in order to modify the aesthetics of a solar module with a coating containing a phase change material. The coating with the phase change material may be applied to a solar glass or solar module. During the curing step, patterns may be generated on the solar glass by locally increasing the temperature on a portion of the coating, such as exposing the coating to hot bars. In another example, conductivity can be changed with addition of a phase change material to the coating, such as to increase an anti-static property.

In an embodiment, an economic factor may be used by the tuning module 2328 to modify the coating selection or the coating process. The tuning module 2328 may attempt to balance the cost/price of the coating or an element thereof with performance. For example, a multi-layer coating may give better performing optical performance but may come with a higher initial cost due to the complexity and materials used in a multi-step process versus a single step process.

In general and in many of the embodiments disclosed herein, to reduce manufacturing complexity, multiple functions (e.g. anti-reflective, abrasion resistant, hydrophobic, biocide, etc.) may be included or enabled in a single coating. In another example, if the input requirement is for an anti-soiling coating, a coating containing a fluorinated compound may be selected, such as by the selection module 2324. However, the input may be filtered by the tuning module 2328 to only those coatings falling under a certain price point. Thus, the selected coating, while still being anti-soiling, may only be selected from lower cost coatings, which may exclude the relatively higher costing fluorinated compounds. The tuning module 2328 may further be used for additional fine-tuning Continuing with this example, even though a cost limitation was placed by the tuning module 2328 on the selection of an anti-soiling coating, the tuning module 2328 may further specify that if the solar module is to be used in an environment that experiences excessive soiling, such as an urban environment, then the restriction to cost may be eliminated or reduced in priority.

As described herein, any of the variables in the requirements specification 2318 may be utilized by any of the modules of the processing system 2320 to select and tune a coating that can be applied at low temperature for an optical element, and one or more databases may be searched to determine data relevant to the selection and/or tuning Various scenarios for selecting and/or tuning a coating, including a coating that can be low temperature coated, exist and may be based on or otherwise involve information obtained about the coating through observation, experimentation, and/or modeling. Exemplary scenarios are described herein. Various parameters may be utilized by any of the modules of the processing system 2320 in selecting or tuning a coating, such as abrasion resistance, refractive index, chemistry (e.g. hydrophobicity tuning by chemistry, hardness tuning by chemistry), optical properties, hardness, and the like.

There may be multiple ways of mitigating mold on solar glass. One way is to make the surface of the solar glass very dry, slippery and non-stick. Alternatively, the surface may be made hydrophilic to attract certain organisms but the coating may include a biocide. Another way to combat mold may be to include a poison in the coating, such as silver or copper. Thus, a coating that imparts such properties or includes such elements might be desired in an environment that is humid or prone to developing mold and might be selected by the processing system 2320.

With respect to soiling, an anti-soiling coating may be a useful selection for certain environments. However, in some extreme environments, such as those where there might be a dust storm, an anti-soiling coating may be insufficient. Indeed, in addition to an anti-soiling coating, or in place of, a coating that might best resist the damage of the soiling itself as well as the clean-up efforts might be selected. Continuing with this example, a solar module placed in an environment prone to dust storms might be preferably recommended to be coated with a hard, abrasion resistant, non-stick coating. Moisture may form a bond between dust and glass, therefore the coating may be selected to keep the surface of the solar module dry such as with a super hydrophobic coating.

With respect to optimizing longevity of the solar glass/module, coating hardness and the UV resistance may be important factors to consider. Coatings that rate well in these categories may be selected by the processing system 2320 if longevity is desired. In embodiments, coatings adapted for longevity may degrade in a benign fashion.

In embodiments, coatings adapted for longevity may be re-activated or reapplied in the field. For example, an anti-reflective coating may be applied that is very dense, non-porous and hydrophobic. The hydrophobicity may weather away over time; however, because the coating is dense, a thin layer of a hydrophobic material (e.g. monolayer of fluorosilane) may be applied to make the surface hydrophobic again for at least a period of time. Continuing with the example, because the coating is hard, it can withstand preparation for field coating. Indeed, the processing system 2320 may enable longitudinal coating planning and tuning of the coating properties versus the capital cost of the overall project. A customer may input specific longitudinal plans for the solar modules and the system 2320 may be able to select coatings that accommodate the plan, taking into account, for example, the cost and availability of personnel to reapply coatings in the field, and the accessibility of the environment in which the coated modules will be deployed. For example, one input, which may be tied to an example of warranty planning, may be a desire for maximum gain for 5 years with a potential trade-off in longevity. Another input may be steady performance for 15 years with a trade-off in maximizing performance. Yet another input may be maximum performance desired after a period of time. For this input, part of the longitudinal coating plan is to apply a coating that is thicker than needed and plan for a wear profile of the coating that delivers later-in-life maximum performance. The processing system 2320 may enable performance timing, such as being able to have solar modules installed at different times and have them reach their maximum performance at the same time. In embodiments, the coating may be optimized for less than maximum performance at the beginning of application and wear could improve performance so that batches of modules could be tuned to peak at the same time. In other embodiments, the coating applied could improve performance over time due to continued curing, such as by UV or heat curing. The processing system 2320 may enable installation of modules at very different times during a project. For example, a solar module installation project may take years to complete. With the desire to have the power profile of the solar modules match, the first installed solar modules may be coated with a coating and the processing system 2320 may indicate that subsequent modules should be coated so that they match the wear profile of the currently installed solar modules. In this case, the first modules may have been coated thicker than required and an abrasion/ablation/wear profile was modeled or planned for such that a later-coated solar module could be matched to the first solar module in terms of performance. In some embodiments, the selected coating may be reactivated periodically in the field such as by removing a weathered portion of the coating. In this embodiment, the coating may be applied thickly with the intent of re-exposing the performing elements of the coating periodically over the life of the solar module. In embodiments, the coating may be adapted to enable re-application of a functional layer in the field. For example, the coating may have exposed hydroxyl groups or easily exposed hydroxyl groups in order to facilitate new siloxane linkages.

With respect to mitigating UV damage of any material on the solar module, the coating and/or the glass may be selected to shift the spectrum transmitted through the glass. The spectral-shifting coating or glass material may absorb UV photons and re-emit them as longer-wavelength photons, so long as the spectral shifting materials have exposure to the solar spectrum. This shift may serve to increase the power of the module if the UV photons can be shifted into the visible range, while also protecting it from damage. In an embodiment, phosphorus systems in the coatings or in the glass itself may be used. Quantum dots, such as tin oxide quantum dots, may be used, which may be tunable to absorb particular frequencies. If the requirements specification 2318 indicates that the glass is UV capturing, the coating may be selected to avoid something that filters UV.

If the requirements specification 2318 is that the coating needs to be applied in the field, certain considerations may be made when selecting a coating and a coating process. For example, less hazardous solvents may be used in the coating formulation. Alternatively, an aqueous-based coating or one that is easier to dispose of may be selected. The selected coating may be tolerant of dirt on the glass, as field-installed solar modules will not be as clean in the field as they are in the factory. The coating may be selected for its ease of application, such as application by simple spraying or wiping. The coating may be selected for its ease of curing, such as curing at environmental temperatures.

If the input specification is that the coating needs to be seasonally optimized, the coating properties may be matched to the season requirements. In an embodiment, a hydrophilic coating may be useful during the summertime. In embodiments, the coating may be selected to include a material that may be removable with the change of the season. For example, anti-soiling in the spring (e.g. pollen) may be a chief concern, and diffuse light capture or anti-reflectivity may be a primary concern in the winter, where a small amount of dirt may actually aid in light capture. A coating that is optimized to work under both these seasonal conditions may include a layer that wipes off or is otherwise removable so that an alternative layer geared for the season may be applied. For example, a springtime coating may include an anti-static property that electrostatically prevents the pollen from sticking. The coating may include a sacrificial layer that gets removed to allow the solar module to be re-coated for non-spring conditions or to expose a fresh surface. In environments where snow and ice are present, a hydrophobic coating may be indicated to cause snow or ice to slide off the solar panel. In other embodiments, the coating may be tuned to resist ice formation. In an example, a solar module may have a conductive front glass and thermally conductive frame. Heat from the frame may be sufficient to loosen the snow. In embodiments, a portion of the infrared component of light striking the surface of the solar module may be utilized in the heating the coding in order to melt the snow. Nanotube fillers and/or nano wires may be utilized in the construction of the solar module in order to mitigate the effects of snow and ice accumulation on the panel.

In embodiments, the anti-static nature of the coating may be tuned by modifying the conductivity of the coating. Hydrophilic coatings are conductive due to the moisture that may be present on the coating. Conductive polymers or metals may be used in anti-static coatings. The conductive polymers may be UV resistant, transparent conducting film made from carbon nanotubes, and the like. Conductive fillers may be used to provide conductivity, which may also serve to increase abrasion resistance.

In some embodiments, conductive coatings on solar modules may enable the active dispersal of dust and other soiling elements. Along with electronics that control the conductivity, conductive coatings may be used to create shifting electric fields to move dust particles from one location on the solar module to another location. Such a system may also be adapted to work in conjunction with another cleaning source, such as a hose, sprinkler, rainfall, and the like. The system may be used with remotely located or otherwise inaccessible solar modules, such as on commercial roofs, microwave transmitters/relay stations, utility poles, and the like.

In embodiments, the photoelectric effect may be sufficient to discharge the surface. For example, a test array may be used in an environment to measure the typical accumulated charge on the module in that environment and a coating may be selected based on the measured accumulated charge. In another embodiment, the solar modules may be designed to intentionally build a charge on the surface in order to dissipate particles.

If the requirements specification is that the coating needs to be able to shed water, such as for solar modules mounted on ocean-going drones or seafaring vessels, so that the salt in the seawater does not build up on the module when the water dries, the processing system may select a coating tuned to this requirement, such as by being hydrophobic.

If the requirements specification is that the coating needs to be able to be applied to a space-residing solar cell, the processing system may select a coating that is adapted to be applied directly to the photovoltaic cell rather than glass. Further, the coating may be adapted to withstand intense ultraviolet radiation and the impact of solar winds.

If the requirements specification relates to a human or economic factor, such as whether or not skilled personnel are available to apply or maintain/clean the coating, the coating may be adapted to accommodate the human or economic factor. For example, in the case where there are no skilled personnel available to apply the coating, the coating may be adapted to be applied in a manner easily done by a layperson. In another example, water may not be available for cleaning so the coating may be adapted to be cleaned with dry brushes, compressed air, vacuum, or the like. In embodiments, the compressed air or vacuum facilities may be powered by the solar module or solar module array itself. In another example, only cheap/salty water may be available to clean the modules, so the coating may be adapted to withstand and/or shed the salty water.

If the requirements specification is that the coating requires a functional element, such as antibiotics, fluorocarbons, quantum dots, metal ions, pigments, dyes, PV elements, phosphors, catalysts (e.g., to create oxygen radicals), the coating may be selected such that the functionalizing component is included and the coating and/or curing process may be selected such that the component is not damaged.

In embodiments, the coating may be a two-layer interference abrasion resistant coating with high refractive index in one layer and low refractive index in another layer that in combination creates disruptive interference to improve performance of the solar module. In embodiments, the requirements specifications 2318 may indicate the existence of one of the layers already and the processing system 2320 may suggest coating with a second layer to create disruptive interference. In embodiments, the second layer may be coating using a flow coating technique, a roll coating technique, or any other coating technique.

Once a particular formulation has been determined to be effective in a certain environment or condition or against a certain agent/environmental insult, customers may be able to bypass the system and instead purchase pre-identified coating formulations. For example, a coating known to be suitable where large avian populations are present may be sold as a coating to mitigate the effects or reduce the adherence of avian feces. In another example, a coating known to be suitable in a humid environment may be sold as a coating to mitigate the effects or reduce the adherence/growth of biofilms and/or mold. In yet another example, a coating known to be suitable in a volcanic environment may be sold as a coating to mitigate the effects of acid rain/SO₂ exposure to the module.

If the input specification is that the coating is to be applied on a solar module to be used at high altitude where the UV flux may be much higher, the coatings may be selected for increased UV resistance such as by including UV filters or by including factors that restore a UV filtering function of the glass. Snow/ice may be an issue at higher altitudes and has been discussed herein. At higher altitudes, the spectrum of light available to the solar module may be different than at lower altitudes, so the coding may be selected to optimize collection of light in that spectrum.

In embodiments, the platform 2300 may be embodied in a user interface. In one embodiment, a coating determination user interface may receive at least one input, user requirement, or process specification, and receive one or more preferences for data, libraries, and inventory to be searched in identifying a candidate coating. The user interface may use a processor to process the at least one input, user requirement, or process specification in accordance with the preference to identify at least one candidate coating. The user interface may display the at least one candidate coating in a ranked list that is sortable by one or more properties or fields (e.g. price, AR, durability). In another embodiment, a coating determination user interface may receive at least one input, user requirement, or process specification and receive one or more preferences for data, libraries, and inventory to be searched in identifying a candidate coating. The user interface may use a processor to process the at least one input, user requirement, or process specification in accordance with the preference to identify at least two candidate coatings. The user interface may display the at least two candidate coatings in a comparison chart that aligns one or more properties or fields of the candidate coatings (e.g. price, AR, durability).

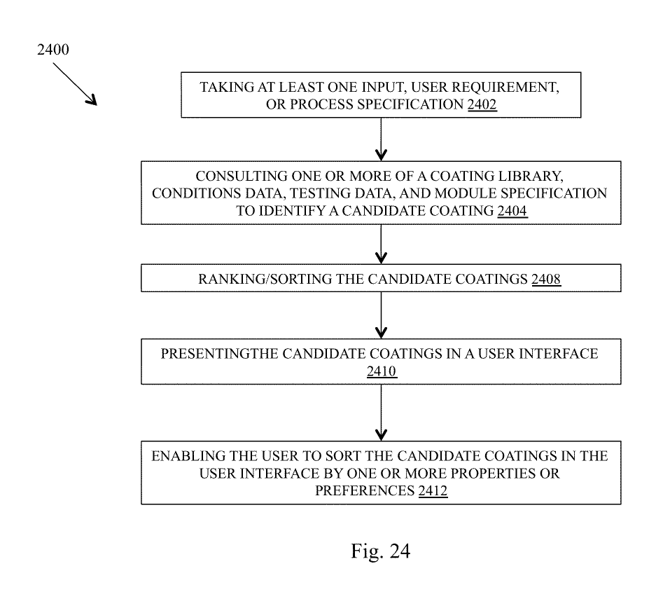

Referring to FIG. 24, a sample workflow 2400 of the platform for determining a coating includes taking at least one input, user requirement, or process specification 2402 at a processing system of the platform and using the processing system to identify a candidate coating. Identifying includes searching one or more of a coating library, conditions data, testing data, and module specification 2404 with the at least one input, user requirement, or process specification. The candidate coatings may be ranked/sorted 2408. The candidate coatings may be presented to a user 2410, such as in a user interface that allows the user to sort the candidate coatings by one or more properties or preferences (e.g. price, AR, durability) 2412. Elements of this workflow, as well as other examples of workflows possible with the platform 2300, are described herein.

The methods and systems described herein may be deployed in part or in whole through a machine that executes computer software, program codes, and/or instructions on a processor 2322. The present disclosure may be implemented as a method on the machine, as a system or apparatus as part of or in relation to the machine, or as a computer program product embodied in a computer readable medium executing on one or more of the machines. In embodiments, the processor may be part of a server, cloud server, client, network infrastructure, mobile computing platform, stationary computing platform, or other computing platform. A processor may be any kind of computational or processing device capable of executing program instructions, codes, binary instructions and the like. The processor may be or may include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more thread. The thread may spawn other threads that may have assigned priorities associated with them; the processor may execute these threads based on priority or any other order based on instructions provided in the program code. The processor, or any machine utilizing one, may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer software on a server, client, firewall, gateway, hub, router, or other such computer and/or networking hardware. The software program may be associated with a server that may include a file server, print server, domain server, internet server, intranet server, cloud server, and other variants such as secondary server, host server, distributed server and the like. The server may include one or more of memories, processors, computer readable media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers, social networks, and the like. Additionally, this coupling and/or connection may facilitate remote execution of program across the network. The networking of some or all of these devices may facilitate parallel processing of a program or method at one or more location without deviating from the scope of the disclosure. In addition, any of the devices attached to the server through an interface may include at least one storage medium capable of storing methods, programs, code and/or instructions. A central repository may provide program instructions to be executed on different devices. In this implementation, the remote repository may act as a storage medium for program code, instructions, and programs.

The software program may be associated with a client that may include a file client, print client, domain client, internet client, intranet client and other variants such as secondary client, host client, distributed client and the like. The client may include one or more of memories, processors, computer readable media, storage media, ports (physical and virtual), communication devices, and interfaces capable of accessing other clients, servers, machines, and devices through a wired or a wireless medium, and the like. The methods, programs, or codes as described herein and elsewhere may be executed by the client. In addition, other devices required for execution of methods as described in this application may be considered as a part of the infrastructure associated with the client.

The client may provide an interface to other devices including, without limitation, servers, other clients, printers, database servers, print servers, file servers, communication servers, distributed servers and the like. Additionally, this coupling and/or connection may facilitate remote execution of program across the network. The networking of some or all of these devices may facilitate parallel processing of a program or method at one or more location without deviating from the scope of the disclosure. In addition, any of the devices attached to the client through an interface may include at least one storage medium capable of storing methods, programs, applications, code and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules and/or components as known in the art. The computing and/or non-computing device(s) associated with the network infrastructure may include, apart from other components, a storage medium such as flash memory, buffer, stack, RAM, ROM and the like. The processes, methods, program codes, instructions described herein and elsewhere may be executed by one or more of the network infrastructural elements. The methods and systems described herein may be adapted for use with any kind of private, community, or hybrid cloud computing network or cloud computing environment, including those which involve features of software as a service (SaaS), platform as a service (PaaS), and/or infrastructure as a service (IaaS).

The methods, program codes, and instructions described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like. The cell network may be a GSM, GPRS, 3G, EVDO, mesh, or other networks types.

The methods, program codes, and instructions described herein and elsewhere may be implemented on or through mobile devices. The mobile devices may include navigation devices, cell phones, mobile phones, mobile personal digital assistants, laptops, palmtops, netbooks, pagers, electronic books readers, music players and the like. These devices may include, apart from other components, a storage medium such as a flash memory, buffer, RAM, ROM and one or more computing devices. The computing devices associated with mobile devices may be enabled to execute program codes, methods, and instructions stored thereon. Alternatively, the mobile devices may be configured to execute instructions in collaboration with other devices. The mobile devices may communicate with base stations interfaced with servers and configured to execute program codes. The mobile devices may communicate on a peer-to-peer network, mesh network, or other communications network. The program code may be stored on the storage medium associated with the server and executed by a computing device embedded within the server. The base station may include a computing device and a storage medium. The storage device may store program codes and instructions executed by the computing devices associated with the base station.

The computer software, program codes, and/or instructions may be stored and/or accessed on machine readable media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g. USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

The methods and systems described herein may transform physical and/or or intangible items from one state to another. The methods and systems described herein may also transform data representing physical and/or intangible items from one state to another.

The elements described and depicted herein, including in flow charts and block diagrams throughout the figures, imply logical boundaries between the elements. However, according to software or hardware engineering practices, the depicted elements and the functions thereof may be implemented on machines through computer executable media having a processor capable of executing program instructions stored thereon as a monolithic software structure, as standalone software modules, or as modules that employ external routines, code, services, and so forth, or any combination of these, and all such implementations may be within the scope of the present disclosure. Examples of such machines may include, but may not be limited to, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices having artificial intelligence, computing devices, networking equipment, servers, routers and the like. Furthermore, the elements depicted in the flow chart and block diagrams or any other logical component may be implemented on a machine capable of executing program instructions. Thus, while the foregoing drawings and descriptions set forth functional aspects of the disclosed systems, no particular arrangement of software for implementing these functional aspects should be inferred from these descriptions unless explicitly stated or otherwise clear from the context. Similarly, it will be appreciated that the various steps identified and described above may be varied, and that the order of steps may be adapted to particular applications of the techniques disclosed herein. All such variations and modifications are intended to fall within the scope of this disclosure. As such, the depiction and/or description of an order for various steps should not be understood to require a particular order of execution for those steps, unless required by a particular application, or explicitly stated or otherwise clear from the context.

The methods and/or processes described above, and steps associated therewith, may be realized in hardware, software or any combination of hardware and software suitable for a particular application. The hardware may include a general-purpose computer and/or dedicated computing device or specific computing device or particular aspect or component of a specific computing device. The processes may be realized in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine-readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software, or any other machine capable of executing program instructions.

Thus, in one aspect, methods described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the present disclosure.

Embodiments described herein are well suited to performing various other steps or variations of the steps recited herein, and in a sequence other than that depicted and/or described herein.

It should be appreciated that reference throughout this specification to “one embodiment” or “an embodiment” means that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the disclosure.

Similarly, it should be appreciated that in the foregoing description of exemplary embodiments of the disclosure, various features of the disclosure are sometimes grouped together in a single embodiment, figure, or description thereof for the purpose of streamlining the disclosure aiding in the understanding of one or more of the various inventive aspects. This method of disclosure, however, is not to be interpreted as reflecting an intention that the claimed disclosure requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this disclosure.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

The use of the terms “a” and “an” and “the” and similar referents in the context of describing the disclosure (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above described embodiment, method, and examples, but by all embodiments and methods within the scope and spirit of the disclosure.

All documents referenced herein are hereby incorporated by reference

What is claimed is:

1. A process for developing a coating specification library, comprising:
gathering geo-located environmental data;
associating particular variables derived from a set of environmental datasets with geographic locations in a single GIS database by importing each environmental dataset as a set of maps;
generating a location genome comprising a set of values for the particular variables associated with the geographic locations;
sorting the locations into a limited number of searchable classification groups based upon their genome values to reduce the very large number of unique location genomes to a smaller number of classes that can then be used to select specific coating properties; and
determining the response of glass with and without various coatings at the geographic locations with selected classifications and storing the coating response in association with at least one of the location genome and location classification group in a coating response database.

2. The process of claim 1, further comprising, optimizing the location genome by filtering locations to include only locations that are suitable for the installation of solar energy generation systems.

3. The process of claim 1, further comprising, optimizing the location genome by ranking locations according to how suitable they are for the installation of solar energy generation systems.

4. The process of claim 1, further comprising, optimizing the location genome by sorting the specific geographic locations by size.

5. The process of claim 1, wherein development of the classification group is by at least one of theory and empirical evidence from field sensors or lab experiments.

6. The process of claim 1, wherein the geo-located environmental data include at least one of climatic classification data, meteorological data, pollution data, soil classification data and biological data.

7. The process of claim 1, wherein the maps can be viewed and manipulated as a stack of layers.

8. The process of claim 7, wherein new layers can be derived by algorithmically combining multiple layers.

9. The process of claim 1, wherein the values describe relevant data for predicting the interactions of glass surfaces with the environment at that geographic location.

10. The process of claim 9, wherein the relevant data further include at least one of measurements of temperature, precipitation, wind, rainfall, snowpack, river flow, time spent below dew point, solar, spectral distribution, air quality index, specific pollutant concentration, SO₂ concentration, dust type/concentration/dust chemistry, alkali lakebed presence, road salt exposure, iron oxide exposure from train tracks, petrochemical/combustion exposure from nearby industry, tire/break debris from nearby roads, seabird population, insect populations, biofilm/mold prone areas, moss, altitude, rivers, lakes, desert, seaside, and sea foam exposure.

11. A coating specification library for coating glass, comprising:
a GIS database including an electronic data set of geo-located environmental data; and
a location genome electronic data structure comprising a set of values for the particular variables associated with the geographic locations, wherein the values provide relevant data for predicting the interactions of glass surfaces with the environment at a geographic location,
wherein the geographic locations are classified into searchable classification groups based upon their genome values.

12. The coating specification library of claim 11, wherein maps based on the GIS database can be viewed and manipulated as a stack of layers.

13. The coating specification library of claim 12, wherein new layers can be derived by algorithmically combining multiple layers.

14. The coating specification library of claim 11, further comprising a module storing data relating to performance of glass using coatings having particular characteristics under particular environmental conditions.

15. The coating specification library of claim 11, wherein the environmental data further include at least one of measurements of temperature, precipitation, wind, rainfall, snowpack, river flow, time spent below dew point, solar, spectral distribution, air quality index, specific pollutant concentration, SO₂ concentration, dust type/concentration/dust chemistry, alkali lakebed presence, road salt exposure, iron oxide exposure from train tracks, petrochemical/combustion exposure from nearby industry, tire/break debris from nearby roads, seabird population, insect populations, biofilm/mold prone areas, moss, altitude, rivers, lakes, desert, seaside, and sea foam exposure.

16. The coating specification library of claim 11, wherein the location genome is filtered to include only locations that are suitable for the installation of solar energy generation systems.

17. The coating specification library of claim 16, wherein the location genome is ranked according to how suitable the locations are for the installation of solar energy generation systems.

18. The coating specification library of claim 11, wherein the location genome is filtered by sorting the specific geographic locations by size.

19. The coating specification library of claim 11, wherein the geo-located environmental data represent at least one of climatic classification data, meteorological data, pollution data, soil classification data and biological data.

20. The coating specification library of claim 11, wherein development of the classification group is by at least one of theory and empirical evidence from field sensors or lab experiments.
